(19) United States
(12) Patent Application Publication
(10) Pub. No.: US 2024/0173257 A1
(43) Pub. Date: May 30, 2024

(54) Liposome formulations

(71) Applicant: Thermosome GmbH, Planegg (DE)

(72) Inventors: Martin Hossann, Planegg (DE); Barbara Cornelia Wedmann, Planegg (DE); Lars Hartwin Lindner, Munich (DE); Wouter Jacobus Marinus Lokerse, Molndal (SE)

(73) Assignee: Thermosome GmbH, Planegg (DE)

(21) Appl. No.: 18/004,182

(22) PCT Filed: Jul. 6, 2021
(86) PCT No.: PCT/EP2021/068568

§ 371 Date: Jan. 4, 2023

(30) Foreign Application Priority Data
Jul. 7, 2020 (EP) 20184542.7

(51) Int. Cl. A61K 9/127; A61K 31/4745; A61K 31/704; A61K 31/7068; A61P 35/00

(52) U.S. Cl. CPC A61K 9/1271; A61K 31/4745; A61K 31/704; A61K 31/7068; A61P 35/00

(57) ABSTRACT

The current invention provides thermosensitive liposomes and formulations thereof preferably for treating, ameliorating, delaying, curing and/or preventing cancer.

### FIELD

The invention relates to the field of thermosensitive liposomes and compositions thereof preferably for the treatment of cancer, more preferably soft tissue sarcoma.

### BACKGROUND OF THE INVENTION

Stimuli-responsive nanocarriers such as thermosensitive liposomes (TSL) have been proposed as drug-delivery systems for various active pharmaceutical ingredients. Yatvin et al. described the first thermosensitive liposome formulation that released a hydrophilic active pharmaceutical ingredient upon heating [1], based on 1,2-dipalmitoyl-sn-glycero-3-phosphocholine (DPPC) and 1,2-distearoyl-sn-glycero-3-phosphocholine (DSPC).

A lysolipid-based thermosensitive liposomes (LTSL) formulation developed by Needham et al. [2] is evaluated human clinical trials)(Thermodox®. This formulation is composed of DPPC, 1-stearoyl-sn-glycero-3-phosphocholine (S-Lyso-PC) and 1,2-distearoyl-sn-glycero-3-phosphoethanolamine-N-methoxy(PEG)-2000 (DSPE-PEG2000). However, this formulation showed unsatisfactory stability in the blood stream.

In order to overcome this stability issue, 1,2-diacyl-sn-glycero-3-phospho-rac-oligo-glycerol (PGn) was developed. In 2004, a phospholipid composition for thermosensitive liposomes DPPC/DSPC/DPPG₂ 50:20:30 (mol/mol) (DPPG₂-TSL₃₀%) was described [3]. This formulation has been extensively compared in vitro to the LTSL formulation. However, it has not been investigated how to achieve long-term storage of these formulations [4,5].

Hence, there is an unmet need in the art to achieve drug delivery systems based on DPPG₂-TSL with a suitable stability to allow industry scale manufacturing and long-term storage. A challenge in preclinical development of thermosensitive liposomal formulations for use in human patients is in particular to achieve a stable and long-term store-able formulation, without affecting the desired instability at temperatures >39° C. for heat-induced localized drug delivery to solid tumors in patients. An optimal formulation has to survive the process of industry-scale manufacturing and needs to be long-term storable without change in quality-critical specifications that can affect therapeutic efficacy and safety of the patient.

### DESCRIPTION OF THE INVENTION

### Thermosensitive Liposome

In a first aspect, the invention provides a thermosensitive liposome comprising a bilayer and an intraliposomal buffer, wherein the molar concentration of 1,2-dipalmitoyl-sn-glycero-3-phosphodiglycerol (DPPG₂) in said bilayer is at least 15 percent, wherein said thermosensitive liposome comprises an active pharmaceutical ingredient, and wherein the molar ratio between said active pharmaceutical ingredient and the lipids comprised in said bilayer is from 0.05 up to 0.3. Such thermosensitive liposomes are referred to in the current application as liposomes according to or of the invention or thermosensitive liposomes according to or of the invention.

Within the context of the present invention, the molar concentration of a lipid in a bilayer refers to the ratio of the molar amount of said lipid to the total molar amount of all lipids comprised in said bilayer, unless explicitly defined otherwise.

A liposome is a spherical system comprising at least one bilayer and an intraliposomal buffer, wherein said intraliposomal buffer is an aqueous solution which is enclosed by said bilayer (see FIG. 1). As well understood by the skilled person, a bilayer comprises two layers of amphiphilic molecules, oriented in such a way that the supramolecular structure of the bilayer is characterized by two hydrophilic surfaces separated by a hydrophobic middle. The intraliposomal buffer of a liposome is any aqueous solution enclosed by the bilayer comprised in said liposome. The extraliposomal buffer is the aqueous solution wherein said liposome is dispersed. It is clear that the intraliposomal buffer contacts the concave hydrophilic surface of the bilayer, whereas the extraliposomal buffer contacts the convex hydrophilic surface of the bilayer.

A liposome may be unilamellar or multilamellar. A unilamellar liposome only contains a single bilayer enclosing an intraliposomal buffer, whereas a multilamellar liposome comprises more than one bilayer. In a multilamellar liposome, the outer bilayer is the bilayer which encloses the other bilayers comprised in said multilamellar liposome. In the context of a multilamellar liposome, the intraliposomal buffer refers to any aqueous solution enclosed by the outer bilayer. Preferably, the bilayers comprised in a multilamellar liposome are present in an essentially concentric configuration. If reference is made to “said bilayer” or “the bilayer” in the context of a multilamellar liposome, the outer bilayer is meant.

Lipids are molecules that are soluble in nonpolar solvents such as hydrocarbons. Unless explicitly stated otherwise, lipids refer herein to amphiphilic or hydrophobic molecules comprised in a bilayer, which are soluble in nonpolar solvents. In this regard, the terms bilayer and lipid bilayer are used interchangeably in the context of this application. In the context of this application, amphiphilic surfactants are lipids. The term lipid preferably only refers to small molecules that are soluble in nonpolar solvents, wherein small molecules have a molecular weight lower than 900 daltons. According to this preferred definition, amphiphilic or hydrophobic proteins, having a molecular weight larger than 900 daltons, comprised in a bilayer are not lipids.

It is clear that a (lipid) bilayer may comprise non-lipid components. A (lipid) bilayer may comprise non-amphiphilic molecules. For example, small hydrophobic molecules could be present in the hydrophobic middle of a bilayer, or hydrophilic peptides could be associated with one of the hydrophilic surfaces of a bilayer.

A liposome according to the invention may be prepared via a method according to the invention, as defined below.

### Thermosensitivity

As well known to the skilled person, a bilayer is not a static structure and the molecules comprised therein are able to move, i.e. to translate and to rotate, in the plane of the bilayer. The fluidity or mobility of a membrane refers to the ease whereby this movement of the molecules in the plane of the bilayer can take place.

A bilayer can exist in a liquid phase (liquid crystalline phase), which is characterized by a high fluidity, or in a solid phase (gel phase, solid gel phase), which is characterized by a low fluidity. The phase in which a bilayer with a specific composition exists in a specific environment is mainly determined by the temperature and the identity of the molecules comprised in said bilayer. In the solid phase, the lipids comprised in said bilayer are ordered in a structured way reminiscent of a crystalline structure, whereas in the liquid phase the lipids are unordered and are able to diffuse freely in the plane of the bilayer.

A bilayer with a specific composition, e.g. consisting of a specific set of lipids in specific molar ratios, will exist in the solid phase if the temperature is lower than the gel to liquid phase transition temperature, and will exist in the liquid phase if the temperature is higher than the gel to liquid phase transition temperature. In the context of this application, the gel to liquid phase transition temperature is also called the transition temperature or Tₘ. The transition temperature is mainly determined by the identity of the molecules comprised in said bilayer, and hence by the lipids comprised in said bilayer.

The transition temperature of a lipid is defined as the transition temperature of a thermosensitive liposome comprising a bilayer, wherein said bilayer essentially consists of said lipid. In this context, “essentially consists” means that the molar concentration of said lipid in said bilayer is at least 95%, or at least 96%, or at least 97%, or at least 98%, or at least 99%, or 100%.

At a temperature around the gel to liquid phase transition temperature, the structure of said bilayer comprises both freely diffusing lipids and rafts of ordered lipids. In other words, the structure of said bilayer is in between the structure of said bilayer at a temperature below the transition temperature and the structure of said bilayer at a temperature above the transition temperature. In the context of this application, the transition temperature range of a bilayer is the range of temperatures wherein said bilayer has such a mixed structure. Evidently, the transition temperature range comprises the transition temperature, and the transition temperature range is mainly determined by the lipids comprised in said bilayer.

The release of an active pharmaceutical ingredient from a thermosensitive liposome according to the invention refers to the transfer of said active pharmaceutical ingredient from said thermosensitive liposome to the extraliposomal buffer. Herein, it is understood that said transfer does not imply the transfer of all molecules of said active pharmaceutical ingredient to the extraliposomal buffer, but implies the transfer of a given molar percentage, as preferably defined below, of said active pharmaceutical ingredient to the extraliposomal buffer. Preferably said active pharmaceutical ingredient is comprised in the intraliposomal buffer of said thermosensitive liposome, and the transfer is from the intraliposomal buffer to the extraliposomal buffer.

In a preferred embodiment the release of an active pharmaceutical ingredient from a thermosensitive liposome means the transfer of at least 50%, at least 55%, at least 60%, at least 65%, at least 70%, at least 75%, at least 80%, at least 85%, at least 90%, at least 90%, at least 91%, at least 92%, at least 93%, at least 94%, at least 95%, at least 96%, at least 97%, at least 97.5%, at least 98%, at least 98.5%, at least 99%, at least 99.5%, or 100% of said active pharmaceutical ingredient from said thermosensitive liposome to the extraliposomal buffer within a given period of time. Preferably, said period of time is 30 minutes, 25 minutes, 20 minutes, 15 minutes, 14 minutes, 13 minutes, 12 minutes, 11 minutes, 10 minutes, 9 minutes, 8 minutes, 7 minutes, 6 minutes, 5 minutes, 285 seconds, 270 seconds, 255 seconds, 240 seconds, 225 seconds, 210 seconds, 195 seconds, 180 seconds, 165 seconds, 150 seconds, 135 seconds, 120 seconds, 105 seconds, 90 seconds, 75 seconds, 60 seconds, 60 seconds, 55 seconds, 50 seconds, 45 seconds, 40 seconds, 35 seconds, 30 seconds, 25 seconds, 20 seconds, 15 seconds, 10 seconds, or 5 seconds.

In a more preferred embodiment the release of an active pharmaceutical ingredient from a thermosensitive liposome means the transfer of at least 75%, at least 80%, at least 85%, at least 90%, at least 90%, at least 91%, at least 92%, at least 93%, at least 94%, at least 95%, at least 96%, at least 97%, at least 97.5%, at least 98%, at least 98.5%, at least 99%, at least 99.5%, or 100% of said active pharmaceutical ingredient from said thermosensitive liposome to the extraliposomal buffer within 10 minutes.

In an even more preferred embodiment the release of an active pharmaceutical ingredient from a thermosensitive liposome means the transfer of at least 75%, at least 80%, at least 85%, at least 90%, at least 90%, at least 91%, at least 92%, at least 93%, at least 94%, at least 95%, at least 96%, at least 97%, at least 97.5%, at least 98%, at least 98.5%, at least 99%, at least 99.5%, or 100% of said active pharmaceutical ingredient from said thermosensitive liposome to the extraliposomal buffer within 5 minutes.

In a most preferred embodiment the release of an active pharmaceutical ingredient from a thermosensitive liposome means the transfer of at least 90% of said active pharmaceutical ingredient from said thermosensitive liposome to the extraliposomal buffer within 5 minutes.

The transfer rate of an active pharmaceutical ingredient quantifies the speed of the transport of said active pharmaceutical ingredient across the bilayer of a thermosensitive liposome. Evidently, the transfer rate may depend on a set of environmental conditions such as the temperature. The release of an active pharmaceutical ingredient refers to the property of a thermosensitive liposome to have a high transfer rate of an active pharmaceutical ingredient under a given set of conditions, preferably above a given temperature (see below). For example, the release of doxorubicin from a thermosensitive liposome according to the invention may only happen at temperatures around the transition temperature of the bilayer comprised in said thermosensitive liposome, allowing the heat-selective delivery of the anti-cancer drug.

As described above, the release of an active pharmaceutical ingredient from a thermosensitive liposome according to the invention is preferably from the intraliposomal buffer to the extraliposomal buffer. In the context of this application, the loading of an active pharmaceutical ingredient is defined as the transfer of said active pharmaceutical ingredient from the extraliposomal buffer to the intraliposomal buffer. The terms release and loading are preferably used with regard to hydrophilic, water-soluble active pharmaceutical ingredients.

Preferably, said release and loading occur via diffusion of said active pharmaceutical ingredient from the intraliposomal buffer to the extraliposomal buffer, or vice versa, whereby said active pharmaceutical ingredient passes the bilayer without active transport. It is well known to the skilled person that active transport of a molecule through or across a (lipid) bilayer means that transport proteins, comprised in or associated with said bilayer, are required for said transfer. It should be noted that active loading does not imply active transport. In the context of this preferred definition, the rate of transfer (via diffusion) depends on the permeability of the bilayer for said active pharmaceutical ingredient. Said permeability depends on the characteristics of said active pharmaceutical ingredient, such as size, polarity, charge, and/or hydrophilicity, and on the leakiness of the bilayer. The leakiness of the bilayer is the inherent permeability of the bilayer. Preferably, an increase or a decrease in the leakiness of a bilayer is defined in this application as an increase or a decrease of the permeability of said bilayer, respectively, for a given active pharmaceutical ingredient with a given charge. More preferably, said active pharmaceutical ingredient is an anti-neoplastic agent, preferably wherein said anti-neoplastic agent is selected from the group consisting of irinotecan, an irinotecan derivative or a pharmaceutically acceptable salt thereof and doxorubicin, a doxorubicin derivative or a pharmaceutically acceptable salt thereof. Most preferably said active pharmaceutical ingredient is doxorubicin, a doxorubicin derivative or a pharmaceutically acceptable salt thereof.

The leakiness of the bilayer comprised in a thermosensitive liposome according to the invention is an important parameter for the use of said thermosensitive liposome in pharmaceutical applications. For example, thermosensitive liposome according to the invention comprising doxorubicin as an active pharmaceutical ingredient could be used as a drug delivery system if the leakiness of the bilayer comprised in said thermosensitive liposome would allow release of said doxorubicin in a patient in need thereof.

A thermosensitive liposome is defined in this application as a liposome comprising an active pharmaceutical ingredient, wherein said active pharmaceutical ingredient is not released at or below a specific temperature, and wherein said active pharmaceutical can be released by increasing the temperature slightly above said temperature. Preferably said temperature is human body temperature. Preferably said specific temperature is from 36° C. up to 40° C., or from 36° C. up to 39° C., or from 36° C. up to 38° C., or from 36.5° C. up to 37.5° C. In this context, “slightly above” means at least 1° C. higher, or at least 1.1° C. higher, or at least 1.2° C. higher, or at least 1.3° C. higher, or at least 1.4° C. higher, or at least 1.5° C. higher, or at least 1.6° C. higher, or at least 1.7° C. higher, or at least 1.8° C. higher, or at least 1.9° C. higher, or at least 2° C. higher, or at least 2.1° C. higher, or at least 2.2° C. higher, or at least 2.3° C. higher, or at least 2.4° C. higher, or at least 2.5° C. higher, or at least 2.6° C. higher, or at least 2.7° C. higher, or at least 2.8° C. higher, or at least 2.9° C. higher, or at least 3° C. higher, or at least 3.1° C. higher, or at least 3.2° C. higher, or at least 3.3° C. higher, or at least 3.4° C. higher, or at least 3.5° C. higher, or at least 3.6° C. higher, or at least 3.7° C. higher, or at least 3.8° C. higher, or at least 3.9° C. higher, or at least 4° C. higher, or at least 4.1° C. higher, or at least 4.2° C. higher, or at least 4.3° C. higher, or at least 4.4° C. higher, or at least 4.5° C. higher, or at least 4.6° C. higher, or at least 4.7° C. higher, or at least 4.8° C. higher, or at least 4.9° C. higher, or at least 5° C. higher.

In a preferred embodiment, a thermosensitive liposome is defined as a liposome wherein said active pharmaceutical ingredient is not released at or below human body temperature, and wherein said active pharmaceutical can be released by increasing the temperature above 39° C., above 39.1° C., above 39.2° C., above 39.3° C., above 39.4° C., above 39.5° C., above 39.6° C., above 39.7° C., above 39.8° C., above 39.9° C., above 40° C., above 40.1° C., above 40.2° C., above 40.3° C., above 40.4° C., above 40.5° C., above 40.6° C., above 40.7° C., above 40.8° C., above 40.9° C., above 41° C., above 41.1° C., above 41.2° C., above 41.3° C., above 41.4° C., above 41.5° C., above 41.6° C., above 41.7° C., above 41.8° C., above 41.9° C., above 42° C., above 42.1° C., above 42.2° C., above 42.3° C., above 42.4° C., above 42.5° C., above 42.6° C., above 42.7° C., above 42.8° C., above 42.9° C., above 43° C., above 43.1° C., above 43.2° C., above 43.3° C., above 43.4° C., above 43.5° C., above 43.6° C., above 43.7° C., above 43.8° C., above 43.9° C., or above 44° C.

In a more preferred embodiment, a thermosensitive liposome is defined as a liposome wherein said active pharmaceutical ingredient is not released at a temperature from 36.5° C. up to 37.5° C., and wherein said active pharmaceutical can be released by increasing the temperature above 40° C.

Without being bound to this theory, the transfer of the active pharmaceutical ingredient from a thermosensitive liposome induced by a slight temperature increase may be caused by the transition of the bilayer comprised in said thermosensitive liposome from the solid to the gel phase. In this context, the transition temperature of said bilayer is slightly above the temperature at or below which said active pharmaceutical ingredient is not released. Above the transition temperature said active pharmaceutical can be released by heating slightly, as defined above. Around the transition temperature, the bilayer comprises both regions which are in the solid phase and regions which are in the liquid phase. At the boundary of these phases, packing defects of the lipids arise, leading to a locally higher diffusion rate of the active pharmaceutical ingredient. In other words, the temperature-induced transfer of the active pharmaceutical ingredient in the thermosensitive liposome may proceed through openings in the lipids bilayer, which arise around the transition temperature of the bilayer due to packing defects. The leakiness of the bilayer is higher around the transition temperature than if the bilayer is fully in the solid phase.

### Active Pharmaceutical Ingredient

An active pharmaceutical ingredient comprised in a thermosensitive liposome according to the invention is, as understood by the skilled person, a molecule which is biologically or clinically active and is responsible for the therapeutic effect (i.e. pharmacodynamic effect) associated with said thermosensitive liposome or with a composition comprising said thermosensitive liposome. In this context, a thermosensitive liposome according to the invention can be envisioned as a drug delivery system for said active pharmaceutical ingredient. Preferably, said therapeutic effect is the treatment, amelioration, delay, cure and/or prevention of cancer, more preferably soft tissue sarcoma.

The purity and the concentration of the active pharmaceutical ingredients comprised in a thermosensitive liposome according to the invention may be determined with HPLC, as described in Example 1.

In a preferred embodiment is provided a thermosensitive liposome according to the invention wherein said active pharmaceutical ingredient is comprised in said intraliposomal buffer.

In another preferred embodiment is provided a thermosensitive liposome according to the invention wherein at least 70%, or at least 80%, or at least 90%, or at least 91%, or at least 92%, or at least 93%, or at least 94%, or at least 95%, or at least 96%, or at least 97%, or at least 98%, or at least 99%, or at least 99.5%, or at least 99.9% of said active pharmaceutical ingredient is comprised in said intraliposomal buffer.

In a preferred embodiment is provided a thermosensitive liposome according to the invention, wherein said active pharmaceutical ingredient is hydrophilic and/or water-soluble. Preferably, water-soluble means a solubility of at least 100 mg/l, 200 mg/l, 300 mg/l, 400 mg/l, 500 mg/l, 600 mg/l, 700 mg/l, 800 mg/l, 900 mg/l, 1000 mg/l, 1500 mg/l, 2000 mg/l, 2500 mg/l, 3000 mg/l, 3500 mg/l, 4000 mg/l, 4500 mg/l, 5000 mg/l, 10 000 g/l, 20 000 g/l, 30 000 g/l, 40 000 g/l, or at least 50 000 g/l in water at 25° C.

In a preferred embodiment is provided a thermosensitive liposome according to the invention, wherein said active pharmaceutical ingredient is an anti-neoplastic agent, preferably wherein said anti-neoplastic agent is comprised in the intraliposomal buffer comprised in said thermosensitive liposome.

In a more preferred embodiment is provided a thermosensitive liposome according to the invention, wherein said active pharmaceutical ingredient is selected from the group consisting of anthracyclines such as doxorubicin, daunorubicin, idarubicin, epirubicin aclarubicin, amrubicin, pirarubicin, valrubicin and zorubicin; anthracenediones such as mitoxantrone and pixantrone; antineoplastic antibiotics such as mitomycin and bleomycin; vinca alkaloids such as vinblastine, vincristine and vinorelbine; alkylating agents such as cyclophosphamide and mechlorethamine hydrochloride; campthothecins such as topotecan, irinotecan (CPT-11), lurtotecan, 9-aminocamptothecin, 9-nitrocamptothecin and 10-hydroxycamptothecin; purine and pyrimidine derivatives such as 5-fluorouracil,gemcitabine (2′,2′-difluoro-2′-deoxycytidine, dFdC), floxuridine (FUDR), cytarabine (cytosine arabinoside), 6-azauracil (6-AU); oxazaphosphorines such as cyclophosphamide, ifosfamide and trofosfamide; taxanes such as paclitaxel and docetaxel; podophyllotoxin derivatives such as etopside and teniposide; platinum-based compounds such as cisplatin, carboplatin, oxaliplatin, nedaplatin; methotrexate; tyrosine kinase inhibitors such as imatinib, gefitinib, erlotinib, sunitinib, adavosertib and lapatinib; and cytarabines such as cytosine arabinoside.

In an even more preferred embodiment is provided a thermosensitive liposome according to the invention wherein said active pharmaceutical ingredient is selected from the group consisting of doxorubicin, daunorubicin, mitoxanthrone, idarubicin, epirubicin, aclarubicin, amrubicin, pirarubicin, valrubicin, zorubicin, pixantrone, mitomycin, bleomycin, vinblastine, vincristine, vinorelbine, cyclophosphamide, mechlorethamine hydrochloride, topotecan, irinotecan, lurtotecan, 9-aminocamptothecin, 9-nitrocamptothecin, 10-hydroxycamptothecin, 5-fluorouracil, gemcitabine, floxuridine, cytarabine, 6-azauracil, cyclophosphamide, ifosfamide, trofosfamide, paclitaxel, docetaxel, etopside, teniposide, cisplatin, carboplatin, oxaliplatin, nedaplatin, methotrexate, imatinib, gefitinib, erlotinib, sunitinib, adavosertib, lapatinib and cytosine arabinoside.

In a most preferred embodiment is provided a thermosensitive liposome according to the invention wherein said active pharmaceutical ingredient is selected from the group consisting of doxorubicin, irinotecan, gemcitabine and pharmaceutically acceptable salts thereof, more preferably said active pharmaceutical ingredient is selected from the group consisting of doxorubicin, irinotecan and pharmaceutically acceptable salts thereof, most preferably said active pharmaceutical ingredient is doxorubicin.

In the context of this application, an active pharmaceutical ingredient refers to the neutral form, and all pharmaceutically acceptable zwitterionic forms, and all pharmaceutically acceptable salts of said active pharmaceutical ingredient. For example, the use of the term doxorubicin herein refers at least to neutral doxorubicin and doxorubicin hydrochloride. As another example, the use of the term irinotecan herein refers at least to neutral irinotecan and irinotecan hydrochloride.

A thermosensitive liposome according to the invention may comprise more than one active pharmaceutical ingredient. Preferably, a thermosensitive liposome according to the invention is provided comprising doxorubicin or a pharmaceutically acceptable salt thereof and/or irinotecan or a pharmaceutically acceptable salt thereof and/or gemcitabine or a pharmaceutically acceptable salt thereof.

In a preferred embodiment is provided a thermosensitive liposome according to the invention wherein said active pharmaceutical ingredient is doxorubicin, a doxorubicin derivative or a pharmaceutically acceptable salt thereof, and wherein the molar ratio between doxorubicin, said doxorubicin derivative or said pharmaceutically acceptable salt thereof and the lipids comprised in said bilayer is from 0.06 up to 0.10, preferably from 0.07 up to 0.09. Preferably, a thermosensitive liposome according to this embodiment is provided,
wherein said thermosensitive liposome has a diameter from 100 nanometers up to 150 nanometers; and/or
wherein said intraliposomal buffer has a pH from 5 up to 8, preferably from 6 up to 8; and/or
wherein said bilayer does not comprise cholesterol or a derivative thereof; and/or
wherein said bilayer comprises a 1,2-dipalmitoyl-sn-glycero-3-phosphocholine (DPPC) and/or 1,2-distearoyl-sn-glycero-3-phosphocholine (DSPC), more preferably
   wherein the molar concentration of 1,2-dipalmitoyl-sn-glycero-3-phosphocholine (DPPC) in said bilayer is from 0.45 up to 0.65, preferably from 0.45 up to 0.55, and/or
   wherein the molar concentration of 1,2-distearoyl-sn-glycero-3-phosphocholine (DSPC) in said bilayer is from 0.15 up to 0.25, and/or
   wherein the molar concentration of 1,2-dipalmitoyl-sn-glycero-3-phosphodiglycerol (DPPG₂) in said bilayer is from 0.15 up to 0.35, preferably from 0.25 up to 0.35; and/or
wherein said 1,2-dipalmitoyl-sn-glycero-3-phosphodiglycerol (DPPG₂) is represented by Formula (IV).

In the context of this application, a doxorubicin derivative is preferably an anthracycline such as daunorubicin, mitoxanthrone, idarubicin, epirubicin, aclarubicin, amrubicin, pirarubicin, valrubicin or zorubicin, or an anthracenedione such as mitoxantrone or pixantrone most preferably a doxorubicin derivative is an anthracycline. In this light, doxorubicin, a doxorubicin derivative or a pharmaceutically acceptable salt thereof is preferably an anthracycline or an anthracenedione or a pharmaceutically acceptable salt thereof, more preferably an anthracycline or a pharmaceutically acceptable salt thereof. Evidently, doxorubicin is considered to be an anthracycline.

In a preferred embodiment is provided a thermosensitive liposome according to the invention wherein said active pharmaceutical ingredient is irinotecan, an irinotecan derivative or a pharmaceutically acceptable salt thereof, and wherein the molar ratio between irinotecan, said irinotecan derivative or said pharmaceutically acceptable salt thereof and the lipids comprised in said bilayer is at least 0.18, preferably at least 0.20. Preferably, a thermosensitive liposome according to this embodiment is provided,
wherein said thermosensitive liposome has a diameter from 100 nanometers up to 150 nanometers; and/or
wherein said intraliposomal buffer has a pH from 5 up to 8, preferably from 6 up to 8; and/or
wherein said bilayer does not comprise cholesterol or a derivative thereof; and/or
wherein said bilayer comprises a 1,2-dipalmitoyl-sn-glycero-3-phosphocholine (DPPC) and/or 1,2-distearoyl-sn-glycero-3-phosphocholine (DSPC), more preferably
   wherein the molar concentration of 1,2-dipalmitoyl-sn-glycero-3-phosphocholine (DPPC) in said bilayer is from 0.45 up to 0.65, preferably from 0.45 up to 0.55, and/or
   wherein the molar concentration of 1,2-distearoyl-sn-glycero-3-phosphocholine (DSPC) in said bilayer is from 0.15 up to 0.25, and/or
   wherein the molar concentration of 1,2-dipalmitoyl-sn-glycero-3-phosphodiglycerol (DPPG₂) in said bilayer is from 0.15 up to 0.35, preferably from 0.25 up to 0.35; and/or
wherein said 1,2-dipalmitoyl-sn-glycero-3-phosphodiglycerol (DPPG₂) is represented by Formula (IV)
wherein the molar ratio between irinotecan, said irinotecan derivative or said pharmaceutically acceptable salt thereof and the lipids comprised in said bilayer is lower than 0.3.

In the context of this application, an irinotecan derivative is preferably a campthothecin such as topotecan, lurtotecan, 9-aminocamptothecin, 9-nitrocamptothecin or 10-hydroxycamptothecin. In this light, irinotecan, an irinotecan derivative or a pharmaceutically acceptable salt thereof is preferably a campthothecin or a pharmaceutically acceptable salt thereof. Evidently, irinotecan is considered to be a campthothecin.

In a preferred embodiment is provided a thermosensitive liposome according to the invention wherein said active pharmaceutical ingredient is gemcitabine, a gemcitabine derivative or a pharmaceutically acceptable salt thereof, and wherein the molar ratio between gemcitabine, said gemcitabine derivative, or said pharmaceutically acceptable salt thereof and the lipids comprised in said bilayer is at least 0.12, preferably at least 0.15. Preferably, a thermosensitive liposome according to this embodiment is provided,
wherein said thermosensitive liposome has a diameter from 100 nanometers up to 150 nanometers; and/or
wherein said intraliposomal buffer has a pH from 5 up to 8, preferably from 6 up to 8; and/or
wherein said bilayer does not comprise cholesterol or a derivative thereof; and/or
wherein said bilayer comprises a 1,2-dipalmitoyl-sn-glycero-3-phosphocholine (DPPC) and/or 1,2-distearoyl-sn-glycero-3-phosphocholine (DSPC), more preferably
   wherein the molar concentration of 1,2-dipalmitoyl-sn-glycero-3-phosphocholine (DPPC) in said bilayer is from 0.45 up to 0.65, preferably from 0.45 up to 0.55, and/or
   wherein the molar concentration of 1,2-distearoyl-sn-glycero-3-phosphocholine (DSPC) in said bilayer is from 0.15 up to 0.25, and/or
   wherein the molar concentration of 1,2-dipalmitoyl-sn-glycero-3-phosphodiglycerol (DPPG₂) in said bilayer is from 0.15 up to 0.35, preferably from 0.25 up to 0.35; and/or
wherein said 1,2-dipalmitoyl-sn-glycero-3-phosphodiglycerol (DPPG₂) is represented by Formula (IV),
preferably wherein the molar ratio between gemcitabine, said gemcitabine derivative, or said pharmaceutically acceptable salt thereof and the lipids comprised in said bilayer is lower than 0.3.

In the context of this application, a gemcitabine derivative is preferably a purine or a pyrimidine derivative such as 5-fluorouracil (5FU), floxuridine (FUDR), cytarabine (cytosine arabinoside) or 6-azauracil (6-AU), more preferably a pyrimidine derivative. In this light, gemcitabine, a gemcitabine derivative or a pharmaceutically acceptable salt thereof is preferably a purine, a pyrimidine derivative or a pharmaceutically acceptable salt thereof, more preferably a pyrimidine derivative of a pharmaceutically acceptable salt thereof. Evidently, gemcitabine is considered to be a pyrimidine derivative.

### Diameter and Zeta Potential of Thermosensitive Liposomes

A thermosensitive liposome according to the invention having a given diameter is a thermosensitive liposome with an area-based particle size, which is larger than 0.95 times said diameter and smaller than 1.05 times said diameter, preferably which is larger than 0.975 times said diameter and smaller than 1.025 times said diameter, even more preferably which is larger than 0.99 times said diameter and smaller than 1.001 times said diameter, most preferably wherein said area-based particle size is equal to said diameter.

The presence of a thermosensitive liposome according to the invention having a given diameter in a population of thermosensitive liposomes may be determined via dynamic light scattering. Preferably, a thermosensitive liposome according to the invention having a given diameter refers to a z average diameter determined via dynamic light scattering. Example 1 describes such a method, wherein a DLS instrument is used (Zetasizer Nano ZS, Malvern Instruments, Worcestershire, United Kingdom). This instrument was calibrated with a Nanosphere™ size standard (125 nm, Thermo Fisher Scientific, Waltham, MA, USA).

In a preferred embodiment a thermosensitive liposome according to the invention is provided wherein said thermosensitive liposome has a diameter of at least 100 nanometers.

In another preferred embodiment is provided a thermosensitive liposome according to the invention wherein said thermosensitive liposome has a diameter from 100 nanometers up to 250 nanometers, or from 100 nanometers up to 200 nanometers, or from 100 nanometers up to 150 nanometers, or from 110 nanometers up to 140 nanometers, or from 115 nanometers up to 135 nanometers, or from 120 nanometers up to 130 nanometers.

In a preferred embodiment is provided a thermosensitive liposome according to the invention wherein said thermosensitive liposome has a diameter from 100 nanometers up to 200 nanometers, wherein the molar ratio between doxorubicin, said doxorubicin derivative or said pharmaceutically acceptable salt thereof and the lipids comprised in said bilayer is from 0.06 up to 0.10, and wherein said intraliposomal buffer has a pH from 5 up to 8.

In a more preferred embodiment is provided a thermosensitive liposome according to the invention wherein said thermosensitive liposome has a diameter from 100 nanometers up to 150 nanometers, wherein the molar ratio between doxorubicin, said doxorubicin derivative or said pharmaceutically acceptable salt thereof and the lipids comprised in said bilayer is from 0.07 up to 0.09, and wherein said intraliposomal buffer has a pH from 6.4 up to 8.0.

In a most preferred embodiment is provided a thermosensitive liposome according to the invention wherein said thermosensitive liposome has a diameter from 100 nanometers up to 150 nanometers. Preferably, a thermosensitive liposome according to this embodiment is provided,
wherein said active pharmaceutical ingredient is selected from the group consisting of doxorubicin, a doxorubicin derivative or a pharmaceutically acceptable salt thereof, irinotecan, an irinotecan derivative or a pharmaceutically acceptable salt thereof, and gemcitabine, a gemcitabine derivative or a pharmaceutically acceptable salt thereof, more preferably wherein said active pharmaceutical ingredient is selected from the group consisting of doxorubicin or a pharmaceutically acceptable salt thereof, irinotecan or a pharmaceutically acceptable salt thereof and gemcitabine or a pharmaceutically acceptable salt thereof, and wherein said active pharmaceutical ingredient is comprised in the intraliposomal buffer, more preferably
   wherein the molar ratio between doxorubicin, said doxorubicin derivative or said pharmaceutically acceptable salt thereof and the lipids comprised in said bilayer is from 0.06 up to 0.10, preferably from 0.07 up to 0.09,
   wherein the molar ratio between irinotecan, said irinotecan derivative or said pharmaceutically acceptable salt thereof and the lipids comprised in said bilayer is at least 0.18, preferably at least 0.20,
   wherein the molar ratio between gemcitabine, said gemcitabine derivative, or said pharmaceutically acceptable salt thereof and the lipids comprised in said bilayer is at least 0.12, preferably at least 0.15; and/or
wherein said intraliposomal buffer has a pH from 5 up to 8, preferably from 6 up to 8; and/or
wherein said bilayer does not comprise cholesterol or a derivative thereof; and/or
wherein said bilayer comprises a 1,2-dipalmitoyl-sn-glycero-3-phosphocholine (DPPC) and/or 1,2-distearoyl-sn-glycero-3-phosphocholine (DSPC), more preferably
   wherein the molar concentration of 1,2-dipalmitoyl-sn-glycero-3-phosphocholine (DPPC) in said bilayer is from 0.45 up to 0.65, preferably from 0.45 up to 0.55, and/or
   wherein the molar concentration of 1,2-distearoyl-sn-glycero-3-phosphocholine (DSPC) in said bilayer is from 0.15 up to 0.25, and/or
   wherein the molar concentration of 1,2-dipalmitoyl-sn-glycero-3-phosphodiglycerol (DPPG₂) in said bilayer is from 0.15 up to 0.35, preferably from 0.25 up to 0.35; and/or
wherein said 1,2-dipalmitoyl-sn-glycero-3-phosphodiglycerol (DPPG₂) is represented by Formula (IV).

A thermosensitive liposome according to the invention having a zeta potential of a given value is a thermosensitive liposome with a zeta potential, which is larger than 0.95 times said value and smaller than 1.05 times said value, preferably which is larger than 0.975 times said value and smaller than 1.025 times said value, even more preferably which is larger than 0.99 times said value and smaller than 1.001 times said value, most preferably wherein said area-based particle size is equal to said value.

The presence of a thermosensitive liposome according to the invention having a given zeta potential in a population of thermosensitive liposomes may be determined via a DLS instrument is used (Zetasizer Nano ZS, Malvern Instruments, Worcestershire, United Kingdom) after dilution of samples in physiological saline.

In a preferred embodiment is provided a thermosensitive liposome according to the invention having a zeta potential from −40 mV up to −10 mV, preferably from −35 mV up to −20 mV.

### Intraliposomal Buffer pH

In a preferred embodiment is provided a thermosensitive liposome according to the invention wherein said intraliposomal buffer has a pH from 5.0 up to 8.0, from 5.5 up to 8.0, from 6.0 up to 8.0, from 6.5 up to 8.0, from 7.0 up to 8.0, or from 7.0 up to 7.5. In the context of this application, pH may be measured by a pH meter, calibrated with a pH reference standard solution in a two point calibration.

In a preferred embodiment is provided a thermosensitive liposome according to the invention wherein said intraliposomal buffer has a pH from 5.0 to 8.0 and wherein the molar ratio between doxorubicin, said doxorubicin derivative or said pharmaceutically acceptable salt thereof and the lipids comprised in said bilayer is from 0.06 up to 0.10, or wherein the molar ratio between irinotecan, said irinotecan derivative or said pharmaceutically acceptable salt thereof and the lipids comprised in said bilayer is at least 0.18, or wherein the molar ratio between gemcitabine, said gemcitabine derivative or said pharmaceutically acceptable salt thereof and the lipids in said bilayer is at least 0.12.

In a more preferred embodiment is provided a thermosensitive liposome according to the invention wherein said intraliposomal buffer has a pH from 6.0 to 8.0 and wherein the molar ratio between doxorubicin, said doxorubicin derivative or said pharmaceutically acceptable salt thereof and the lipids comprised in said bilayer is from 0.07 up to 0.09, or wherein the molar ratio between irinotecan, said irinotecan derivative or said pharmaceutically acceptable salt thereof and the lipids comprised in said bilayer is at least 0.20, or wherein the molar ratio between gemcitabine, said gemcitabine derivative or said pharmaceutically acceptable salt thereof and the lipids comprised in said bilayer is at least 0.15.

In a more preferred embodiment is provided a thermosensitive liposome according to the invention wherein said intraliposomal buffer has a pH from 6.0 up to 8.0. Preferably, a thermosensitive liposome according to this embodiment is provided,
wherein said wherein said thermosensitive liposome has a diameter from 100 nanometers up to 150 nanometers; and/or
wherein said active pharmaceutical ingredient is selected from the group consisting of doxorubicin, said doxorubicin derivative or a pharmaceutically acceptable salt thereof, irinotecan, an irinotecan derivative or a pharmaceutically acceptable salt thereof, and gemcitabine, a gemcitabine derivative or a pharmaceutically acceptable salt thereof, more preferably wherein said active pharmaceutical ingredient is selected from the group consisting of doxorubicin or a pharmaceutically acceptable salt thereof, irinotecan or a pharmaceutically acceptable salt thereof and gemcitabine or a pharmaceutically acceptable salt thereof, and wherein said active pharmaceutical ingredient is comprised in the intraliposomal buffer, more preferably
   wherein the molar ratio between doxorubicin, said doxorubicin derivative or said pharmaceutically acceptable salt thereof and the lipids comprised in said bilayer is from 0.06 up to 0.10, preferably from 0.07 up to 0.09,
   wherein the molar ratio between irinotecan, said irinotecan derivative or said pharmaceutically acceptable salt thereof and the lipids comprised in said bilayer is at least 0.18, preferably at least 0.20,
   wherein the molar ratio between gemcitabine, said gemcitabine derivative or said pharmaceutically acceptable salt thereof and the lipids comprised in said bilayer is at least 0.12, preferably at least 0.15; and/or
wherein said bilayer does not comprise cholesterol or a derivative thereof; and/or
wherein said bilayer comprises a 1,2-dipalmitoyl-sn-glycero-3-phosphocholine (DPPC) and/or 1,2-distearoyl-sn-glycero-3-phosphocholine (DSPC), more preferably
   wherein the molar concentration of 1,2-dipalmitoyl-sn-glycero-3-phosphocholine (DPPC) in said bilayer is from 0.45 up to 0.65, preferably from 0.45 up to 0.55, and/or
   wherein the molar concentration of 1,2-distearoyl-sn-glycero-3-phosphocholine (DSPC) in said bilayer is from 0.15 up to 0.25, and/or
   wherein the molar concentration of 1,2-dipalmitoyl-sn-glycero-3-phosphodiglycerol (DPPG₂) in said bilayer is from 0.15 up to 0.35, preferably from 0.25 up to 0.35; and/or
wherein said 1,2-dipalmitoyl-sn-glycero-3-phosphodiglycerol (DPPG₂) is represented by Formula (IV).

### Lipid Composition of the Bilayer

The composition of the bilayer comprised in a thermosensitive liposome according to the invention determines the characteristics of the temperature-induced release and of the stability of said thermosensitive liposome, and hence determines the pharmacokinetic properties of said thermosensitive liposome. Both the identity of the lipids comprised in said bilayer and their ratios are important in this regard. As described in Example 1, the composition of the bilayer can be determined with TLC, and the purity and the concentrations of the lipids comprised in the bilayer can be determined by HPLC.

A thermosensitive liposome according to the invention has a bilayer comprising DPPG₂. Without being bound to this theory, the presence of DPPG₂, having a highly hydrate digylcerol group, in said bilayer inhibits interactions with blood components sterically, both via electrostatic and hydrophobic interactions, thereby rendering said thermosensitive liposome according to the invention non-toxic, as defined below.

In a preferred embodiment is provided a thermosensitive liposome according to the invention wherein the molar concentration of said DPPG₂ in said bilayer is at least 15%, or at least 16%, or at least 17%, or at least 18%, or at least 19%, or at least 20%, or at least 21%, or at least 22%, or at least 23%, or at least 24%, or at least 25%, or at least 26%, or at least 27%, or at least 28%, or at least 29%, or at least 30%, or at least 31%, or at least 32%, or at least 33%, or at least 34%, or at least 35%.

In a preferred embodiment is provided a thermosensitive liposome according to the invention wherein the molar concentration of DPPG₂ in said bilayer is lower than 55%, or lower than 54%, or lower than 53%, or lower than 52%, or lower than 51%, or lower than 50%, or lower than 49%, or lower than 48%, or lower than 47%, or lower than 46%, or lower than 45%, or lower than 44%, or lower than 43%, or lower than 42%, or lower than 41%, or lower than 40%, or lower than 39%, or lower than 38%, or lower than 37%, or lower than 36%, or lower than 35%, or lower than 34%, or lower than 33%, or lower than 32%, or lower than 31%, or lower than 30%, or lower than 29%, or lower than 28%, or lower than 27%, or lower than 26%, or lower than 25%, or lower than 24%, or lower than 23%, or lower than 22%, or lower than 21%, or lower than 20%, or lower than 19%, or lower than 18%, or lower than 17%, or lower than 16%.

In a preferred embodiment is provided a thermosensitive liposome according to the invention wherein the concentration of said DPPG₂ in said bilayer is at least 15%, or from 15% up to 55%, or from 15% up to 54%, or from 15% up to 53%, or from 15% up to 52%, or from 15% up to 51%, or from 15% up to 50%, or from 15% up to 49%, or from 15% up to 48%, or from 15% up to 47%, or from 15% up to 46%, or from 15% up to 45%, or from 15% up to 44%, or from 15% up to 43%, or from 15% up to 42%, or from 15% up to 41%, or from 15% up to 40%, or from 15% up to 39%, or from 15% up to 38%, or from 15% up to 37%, or from 15% up to 36%, or from 15% up to 35%, or from 15% up to 34%, or from 15% up to 33%, or from 15% up to 32%, or from 15% up to 31%, or from 15% up to 30%, or from 15% up to 29%, or from 15% up to 28%, or from 15% up to 27%, or from 15% up to 26%, or from 15% up to 25%, or from 15% up to 24%, or from 15% up to 23%, or from 15% up to 22%, or from 15% up to 21%, or from 15% up to 20%, or from 15% up to 19%, or from 15% up to 18%, or from 15% up to 17%, or from 15% up to 16%.

In a preferred embodiment is provided a thermosensitive liposome according to the invention, wherein the concentration of said DPPG₂ in said bilayer is from 15% up to 40%, from 16% up to 40%, from 17% up to 40%, from 18% up to 40%, from 19% up to 40%, from 20% up to 40%, from 21% up to 40%, from 22% up to 40%, from 23% up to 40%, from 24% up to 40%, from 25% up to 40%, from 26% up to 40%, from 27% up to 40%, from 28% up to 40%, from 29% up to 40%, from 30% up to 40%, from 15% up to 35%, from 16% up to 35%, from 17% up to 35%, from 18% up to 35%, from 19% up to 35%, from 20% up to 35%, from 21% up to 35%, from 22% up to 35%, from 23% up to 35%, from 24% up to 35%, from 25% up to 35%, from 26% up to 35%, from 27% up to 35%, from 28% up to 35%, from 29% up to 35%, from 30% up to 35%.

A bilayer comprised in a thermosensitive liposome according to the invention preferably comprises phospholipids, more preferably glycerophospholids.

In a preferred embodiment is provided a thermosensitive liposome according to the invention wherein said bilayer comprises 1,2-diacyl-sn-glycero-3-phosphatidylethanolamine (PE), 1,2-diacyl-sn-glycero-3-phosphatidylcholine (PC), 1,2-diacyl-sn-glycero-3-phosphatidylserine (PS), 1,2-diacyl-sn-glycero-3-phosphatidylinositol (PI), 1,2-diacyl-sn-glycero-3-phosphatidylinositol phosphate (PIP), 1,2-diacyl-sn-glycero-3-phosphatidylinositol biphosphate (PIP2), 1,2-diacyl-sn-glycero-3-phosphatidylinositol triphosphate (PIP3), 1,2-diacyl-sn-glycero-3-phosphatidylglycerol (PG), 1,2-diacyl-sn-glycero-3-phosphatidyldiglycerol (PG₂), 1,2-diacyl-sn-glycero-3-phosphatidyltriglycerol (PG₃), and/or 1,2-diacyl-sn-glycero-3-phosphatidyltetraglycerol (PG₄). As explained above, a thermosensitive liposome according to the invention comprises 1,2-dipalmitoyl-sn-glycero-3-phosphatidyldiglycerol (DPPG₂), a specific type of PG₂ by definition.

In a more preferred embodiment is provided a thermosensitive liposome according to the invention wherein 1,2-diacyl-sn-glycero-3-phosphatidylcholine (PC), 1,2-diacyl-sn-glycero-3-phosphatidylethanolamine (PE) and 1,2-dipalmitoyl-sn-glycero-3-phosphatidyldiglycerol (DPPG₂) have a combined concentration of at least 50 mol %, at least 60 mol %, at least 70 mol %, at least 80 mol %, at least 90 mol %, at least 90 mol %, at least 91 mol %, at least 92 mol %, at least 93 mol %, at least 94 mol %, at least 95 mol %, at least 96 mol %, at least 97 mol %, at least 98 mol %, or at least 99 mol % comprised in said bilayer.

In a more preferred embodiment is provided a thermosensitive liposome according to the invention wherein 1,2-diacyl-sn-glycero-3-phosphatidylcholine (PC) and 1,2-dipalmitoyl-sn-glycero-3-phosphatidyldiglycerol (DPPG₂) have a combined concentration of at least 50 mol %, at least 60 mol %, at least 70 mol %, at least 80 mol %, at least 90 mol %, at least 90 mol %, at least 91 mol %, at least 92 mol %, at least 93 mol %, at least 94 mol %, at least 95 mol %, at least 96 mol %, at least 97 mol %, at least 98 mol %, or at least 99 mol % in said bilayer.

The (glycero)phospholipids described herein may be covalently attached to a polyethylene glycol (PEG), a glycerol or a polyglycerol in order to increase the molecular weight and the molecular size of the hydrophilic part of said (glycero)phospholipids. In the case of PEG, such a (glycerol)phospholipid is said to have been PEGylated. For example, DSPE-PEG₂₀₀₀ is N-methoxy(PEG)-2000-1,2-diacyl-sn-glycero-3-phosphatidylethanolamine (PE). Without being bound to this theory, the presence of PEGylated (glycerol)phospholipids in a thermosensitive liposome has a similar effect as the presence of DPPG₂ described above, namely the steric inhibition of interactions with blood component.

In a preferred embodiment is provided a thermosensitive liposome according to the invention wherein said bilayer does not comprise PEG. In other words such a bilayer does not comprise PEGylated lipids.

An important advantage of the use of DPPG₂ over PEGylated lipids is the significantly smaller head group modification of the phospholipid anchor (74 Da for each glycerol unit versus approximately 2000 Da). As result, 1,2-dipalmitoyl-sn-glycero-3-phospho-diglycerol (DPPG₂) forms lamellar instead of micellar structures and could therefore be incorporated into thermosensitive liposome up to 70 mol %. On the other hand, DSPE-PEG2000 incorporation is limited, since it acts like a surfactant with a critical micellar concentration of 0.5-1.0 μM and compromises the membrane bilayer stability at high concentrations.

In the (glycerol)phospholipids mentioned above preferred acyl groups are C14-C22 acyl groups, more preferably C18-20 acyl groups. Preferably, an acyl group is selected from the group consisting of arachidyl, arachyl, behenyl, brassidyl, ceryl, cetyl, elaidyl, erucyl, gadoleyl, geddyl, isostearyl, lauryl, lignoceryl, montanyl, myricyl, myristyl, oleyl, palmitoleyl, palmitoyl, petroselinyl and stearyl. More preferably, acyl groups are selected from the group consisting of stearyl and palmitoyl. Most preferably, an acyl group is palmitoyl.

In a preferred embodiment is provided a thermosensitive liposome according to the invention wherein said bilayer comprises 1,2-dipalmitoyl-sn-glycero-3-phosphatidylcholine (DPPC), 1,2-dipalmitoyl-sn-glycero-3-phosphatidylethanolamine (DPPE), 1,2-distearyl-sn-glycero-3-phosphatidylcholine (DSPC), 1,2-distearyl-sn-glycero-3-phosphatidylethanolamine (DSPE), N-methoxy(PEG)-2000-1,2-distearyl-sn-glycero-3-phosphatidylethanolamine (DSPE-PEG₂₀₀₀), and/or 1,2-dipalmitoyl-sn-glycero-3-phosphatidyldiglycerol (DPPG₂).

In a preferred embodiment is provided a thermosensitive liposome according to the invention wherein 1,2-dipalmitoyl-sn-glycero-3-phosphatidylcholine (DPPC), 1,2-dipalmitoyl-sn-glycero-3-phosphatidylethanolamine (DPPE), 1,2-distearyl-sn-glycero-3-phosphatidylcholine (DSPC), 1,2-distearyl-sn-glycero-3-phosphatidylethanolamine (DSPE), N-methoxy(PEG)-2000-1,2-distearyl-sn-glycero-3-phosphatidylethanolamine (DSPE-PEG₂₀₀₀), and 1,2-dipalmitoyl-sn-glycero-3-phosphatidyldiglycerol (DPPG₂) have a combined concentration of at least 50 mol %, at least 60 mol %, at least 70 mol %, at least 80 mol %, at least 90 mol %, at least 91 mol %, at least 92 mol %, at least 93 mol %, at least 94 mol %, at least 95 mol %, at least 96 mol %, at least 97 mol %, at least 98 mol %, or at least 99 mol % in said bilayer. In the context of this embodiment, it is understood that not all lipids mentioned above may, but should not be comprised in said thermosensitive liposome.

In a preferred embodiment is provided a thermosensitive liposome according to the invention wherein said bilayer comprises 1,2-dipalmitoyl-sn-glycero-3-phosphatidylcholine (DPPC), 1,2-distearyl-sn-glycero-3-phosphatidylcholine (DSPC), 1,2-distearyl-sn-glycero-3-phosphatidylethanolamine (DSPE), N-methoxy(PEG)-2000-1,2-distearyl-sn-glycero-3-phosphatidylethanolamine (DSPE-PEG₂₀₀₀), and/or 1,2-dipalmitoyl-sn-glycero-3-phosphatidyldiglycerol (DPPG₂).

In a preferred embodiment is provided a thermosensitive liposome according to the invention wherein 1,2-dipalmitoyl-sn-glycero-3-phosphatidylcholine (DPPC), 1,2-distearyl-sn-glycero-3-phosphatidylcholine (DSPC), 1,2-distearyl-sn-glycero-3-phosphatidylethanolamine (DSPE), N-methoxy(PEG)-2000-1,2-distearyl-sn-glycero-3-phosphatidylethanolamine (DSPE-PEG₂₀₀₀), and 1,2-dipalmitoyl-sn-glycero-3-phosphatidyldiglycerol (DPPG₂) have a combined concentration of at least 50 mol %, at least 60 mol %, at least 70 mol %, at least 80 mol %, at least 90 mol %, at least 91 mol %, at least 92 mol %, at least 93 mol %, at least 94 mol %, at least 95 mol %, at least 96 mol %, at least 97 mol %, at least 98 mol %, or at least 99 mol % in said bilayer. In the context of this embodiment, it is understood that not all lipids mentioned above may, but should not be comprised in said thermosensitive liposome.

In a preferred embodiment is provided a thermosensitive liposome according to the invention wherein said bilayer comprises 1,2-dipalmitoyl-sn-glycero-3-phosphocholine (DPPC) and/or 1,2-distearoyl-sn-glycero-3-phosphocholine (DSPC). As is clear to the skilled person, DPPG₂ refers to all compounds represented by Formula (I); DPPC refers to all compounds represented by Formula (II); DSPC refers to all compounds represented by Formula (III).

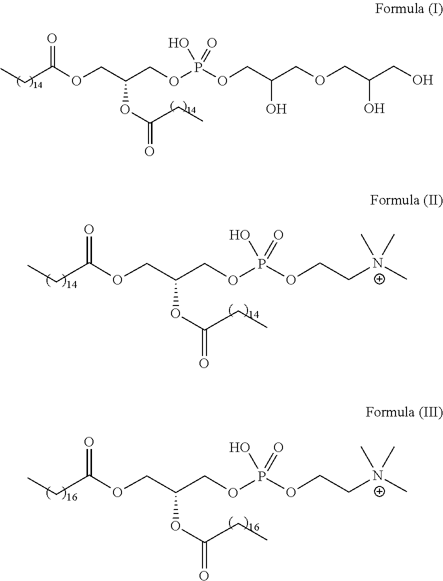

Unless explicitly stated otherwise, a Lewis structure or formula, a trivial name or a systematic name for a molecule is meant to encompass all tautomers, i.e. structural isomers which are readily interconverted, and stereoisomers of these molecules in the context of this application. For example, Formula (I) comprises three chiral carbon centers. The stereoconfiguration of one of these chiral carbon centers has been explicitly defined, and should be interpreted as such. The stereoconfiguration of the other two chiral carbon centers has not been specified. Hence, all epimers with regard to these two chiral carbon centers are described by Formula (I). It should also be noted that, a Lewis structure or formula, a trivial name or a systematic name for a molecule is meant to encompass all protonation states and salts, unless explicitly stated otherwise. For example, Formula (I) also describes DPPG₂ wherein one of the oxygen atoms covalently attached to phosphor is negatively charged.

In a preferred embodiment is provided a thermosensitive liposome according to the invention wherein said 1,2-dipalmitoyl-sn-glycero-3-phosphodiglycerol (DPPG₂) is represented by Formula (IV):

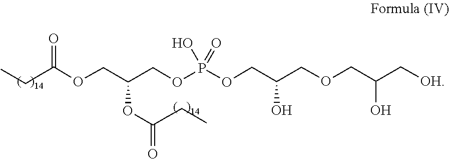

In a preferred embodiment is provided a thermosensitive liposome according to the invention wherein 1,2-dipalmitoyl-sn-glycero-3-phosphatidylcholine (DPPC), 1,2-distearyl-sn-glycero-3-phosphatidylcholine (DSPC), and 1,2-dipalmitoyl-sn-glycero-3-phosphatidyldiglycerol (DPPG₂) have a combined concentration of at least 50 mol %, at least 60 mol %, at least 70 mol %, at least 80 mol %, at least 90 mol %, at least 91 mol %, at least 92 mol %, at least 93 mol %, at least 94 mol %, at least 95 mol %, at least 96 mol %, at least 97 mol %, at least 98 mol %, or at least 99 mol %, or at least 99.5 mol %, or at least 99.6 mol %, or at least 99.7 mol %, or at least 99.8 mol %, or at least 99.9 mol % in said bilayer. In the context of this embodiment, it is understood that not all lipids mentioned above may, but should not be comprised in said thermosensitive liposome.

In a preferred embodiment is provided a thermosensitive liposome according to the invention wherein said bilayer consists of 1,2-dipalmitoyl-sn-glycero-3-phosphatidylcholine (DPPC), 1,2-distearyl-sn-glycero-3-phosphatidylcholine (DSPC), and 1,2-dipalmitoyl-sn-glycero-3-phosphatidyldiglycerol (DPPG₂).

The purity of a thermosensitive liposome according to the invention is the combined concentration of 1,2-dipalmitoyl-sn-glycero-3-phosphatidylcholine (DPPC), 1,2-distearyl-sn-glycero-3-phosphatidylcholine (DSPC), and 1,2-dipalmitoyl-sn-glycero-3-phosphatidyldiglycerol (DPPG₂) in said bilayer. Hence, in a preferred embodiment is provided a thermosensitive liposome according to the invention with a purity of at least 50 mol %, at least 60 mol %, at least 70 mol %, at least 80 mol %, at least 90 mol %, at least 91 mol %, at least 92 mol %, at least 93 mol %, at least 94 mol %, at least 95 mol %, at least 96 mol %, at least 97 mol %, at least 98 mol %, or at least 99 mol %, or at least 99.5 mol %, or at least 99.6 mol %, or at least 99.7 mol %, or at least 99.8 mol %, or at least 99.9 mol %, or 100 mol %.

In a preferred embodiment is provided a thermosensitive liposome according to the invention wherein 1,2-dipalmitoyl-sn-glycero-3-phosphatidylcholine (DPPC), 1,2-distearyl-sn-glycero-3-phosphatidylcholine (DSPC), and 1,2-dipalmitoyl-sn-glycero-3-phosphatidyldiglycerol (DPPG₂) combined constitute at least 95% of the number of molecules comprised in said bilayer, and
wherein said wherein said thermosensitive liposome has a diameter from 100 nanometers up to 150 nanometers; and/or
wherein said active pharmaceutical ingredient is selected from the group consisting of doxorubicin, a doxorubicin derivative or a pharmaceutically acceptable salt thereof, irinotecan, an irinotecan derivative or a pharmaceutically acceptable salt thereof, and gemcitabine, a gemcitabine derivative or a pharmaceutically acceptable salt thereof, and wherein said active pharmaceutical ingredient is comprised in the intraliposomal buffer, more preferably
   wherein the molar ratio between doxorubicin, said doxorubicin derivative or said pharmaceutically acceptable salt thereof and the lipids comprised in said bilayer is from 0.06 up to 0.10, preferably from 0.07 up to 0.09,
   wherein the molar ratio between irinotecan, said irinotecan derivative or said pharmaceutically acceptable salt thereof and the lipids comprised in said bilayer is at least 0.18, preferably at least 0.20,
   wherein the molar ratio between gemcitabine, said gemcitabine derivative or said pharmaceutically acceptable salt thereof and the lipids comprised in said bilayer is at least 0.12, preferably at least 0.15, and/or
wherein said intraliposomal buffer has a pH from 5 up to 8, preferably from 6 up to 8; and/or
wherein said bilayer does not comprise cholesterol or a derivative thereof; and/or
wherein said 1,2-dipalmitoyl-sn-glycero-3-phosphodiglycerol (DPPG₂) is represented by Formula (IV).

It is understood that, if the combined concentration of a group of lipids in a bilayer is a given mol %, said bilayer may but should not comprise all types of lipids in said group of lipids. For example, a bilayer wherein the concentration of DPPC and DSPC is each 45 mol %, may be described as a bilayer wherein the combined concentration of DPPC, DSPC and DPPG₂ is at least 90 mol %.

In a preferred embodiment is provided a thermosensitive liposome according to the invention wherein said bilayer comprises 1,2-dipalmitoyl-sn-glycero-3-phosphocholine (DPPC) and/or 1,2-distearoyl-sn-glycero-3-phosphocholine (DSPC), wherein the molar concentration of 1,2-dipalmitoyl-sn-glycero-3-phosphocholine (DPPC) in said bilayer is from 0.45 up to 0.65, preferably from 0.45 up to 0.55; and/or wherein the molar concentration of 1,2-distearoyl-sn-glycero-3-phosphocholine (DSPC) in said bilayer is from 0.15 up to 0.25; and/or wherein the molar concentration of 1,2-dipalmitoyl-sn-glycero-3-phosphodiglycerol (DPPG₂) in said bilayer is from 0.15 up to 0.35, preferably from 0.25 up to 0.35.

In a preferred embodiment is provided a thermosensitive liposome according to the invention wherein said bilayer comprises 1,2-dipalmitoyl-sn-glycero-3-phosphocholine (DPPC) and 1,2-distearoyl-sn-glycero-3-phosphocholine (DSPC), wherein the molar concentration of 1,2-dipalmitoyl-sn-glycero-3-phosphocholine (DPPC) in said bilayer is from 0.45 up to 0.65, preferably from 0.45 up to 0.55; and/or wherein the molar concentration of 1,2-distearoyl-sn-glycero-3-phosphocholine (DSPC) in said bilayer is from 0.15 up to 0.25; and wherein the molar concentration of 1,2-dipalmitoyl-sn-glycero-3-phosphodiglycerol (DPPG₂) in said bilayer is from 0.15 up to 0.35, preferably from 0.25 up to 0.35, preferably
wherein said wherein said thermosensitive liposome has a diameter from 100 nanometers up to 150 nanometers; and/or
wherein said active pharmaceutical ingredient is selected from the group consisting of doxorubicin, a doxorubicin derivative or a pharmaceutically acceptable salt thereof, irinotecan, an irinotecan derivative or a pharmaceutically acceptable salt thereof, and gemcitabine, a gemcitabine derivative or a pharmaceutically acceptable salt thereof, more preferably wherein said active pharmaceutical ingredient is selected from the group consisting of doxorubicin or a pharmaceutically acceptable salt thereof, irinotecan or a pharmaceutically acceptable salt thereof and irinotecan or a pharmaceutically acceptable salt thereof, and wherein said active pharmaceutical ingredient is comprised in the intraliposomal buffer, more preferably
   wherein the molar ratio between doxorubicin, said doxorubicin derivative or said pharmaceutically acceptable salt thereof and the lipids comprised in said bilayer is from 0.06 up to 0.10, preferably from 0.07 up to 0.09,
   wherein the molar ratio between irinotecan, said irinotecan derivative, or said pharmaceutically acceptable salt thereof and the lipids comprised in said bilayer is at least 0.18, preferably at least 0.20,
   wherein the molar ratio between gemcitabine, said gemcitabine derivative or said pharmaceutically acceptable salt thereof and the lipids comprised in said bilayer is at least 0.12, preferably at least 0.15, and/or
wherein said intraliposomal buffer has a pH from 5 up to 8, preferably from 6 up to 8; and/or
wherein said bilayer does not comprise cholesterol or a derivative thereof; and/or
wherein said 1,2-dipalmitoyl-sn-glycero-3-phosphodiglycerol (DPPG₂) is represented by Formula (IV).

DPPC is the major component in a thermosensitive liposome according to the preferred embodiment above, because its transition temperature (41.4° C.) is slightly above body temperature. Unwanted drug leakage at body temperature, as defined below, can be reduced by mixing DPPC with small amounts of DSPC, which has a transition temperature of 54.9° C.

A lysolipid is an amphiphilic molecule which is the product of the hydrolysis of a lipid. For example, 1-palmitoyl-sn-glycero-3-phosphatidylcholine, resulting from the hydrolysis of the 2-ester bond in DPPC, is a lysolipid. Preferably, a lysolipid is a monoacyl-sn-glycero-3-phosphatidylcholine, a monoacyl-sn-glycero-3-phosphatidylglycerol, or a monoacyl-sn-glycero-3-phosphatidyldiglycerol.

Without being bound to this theory, lysolipids affect the transition temperature of bilayer comprised in a thermosensitive liposome according to the invention, thereby influencing the heat-induced release of the active pharmaceutical ingredient comprised in said thermosensitive liposome. This was shown for thermosensitive liposome according to the invention in Example 5. Therefore, a low concentration of lysolipids is preferred in a thermosensitive liposome according to the invention, preferably lower than 5 mol %, lower than 4 mol %, lower than 3 mol %, lower than 2 mol %, lower than 1 mol %, lower than 1 mol %, lower than 0.9 mol %, lower than 0.8 mol %, lower than 0.7 mol %, lower than 0.6 mol %, lower than 0.5 mol %, lower than 0.4 mol %, lower than 0.3 mol %, lower than 0.2 mol %, or lower than 0.1 mol %.

DPPGₙ phospholipids like DPPG₂ are more prone to decomposition than standard phospholipids like DPPC and DSPC. This increases the challenge to stabilize thermosensitive liposome according to the invention and composition according to the invention compared to other formulations like e.g. LTSL. The stability of a thermosensitive liposome according to the invention and a composition according to the invention is discussed below. It should further be added that an active pharmaceutical ingredient comprised in said thermosensitive liposome or composition might facilitate or induce the formation of lysolipids.

In a preferred embodiment is provided a thermosensitive liposome according to the invention wherein the concentration of lysolipids comprised in the bilayer comprised in said thermosensitive liposome is lower than 5 mol %, lower than 4 mol %, lower than 3 mol %, lower than 2 mol %, lower than 1 mol %, lower than 1 mol %, lower than 0.9 mol %, lower than 0.8 mol %, lower than 0.7 mol %, lower than 0.6 mol %, lower than 0.5 mol %, lower than 0.4 mol %, lower than 0.3 mol %, lower than 0.2 mol %, or lower than 0.1 mol %.

In a preferred embodiment is provided a thermosensitive liposome according to the invention wherein the concentration of lysolipids comprised in the bilayer comprised in said thermosensitive liposome is lower than 2 mol %, preferably lower than 1 mol %, and
wherein 1,2-dipalmitoyl-sn-glycero-3-phosphatidylcholine (DPPC), 1,2-distearyl-sn-glycero-3-phosphatidylcholine (DSPC), and 1,2-dipalmitoyl-sn-glycero-3-phosphatidylglycerol (DPPG₂) combined constitute at least 95% of the number of molecules comprised in said bilayer; and/or
wherein said wherein said thermosensitive liposome has a diameter from 100 nanometers up to 150 nanometers; and/or
wherein said active pharmaceutical ingredient is selected from the group consisting of doxorubicin, a doxorubicin derivative or a pharmaceutically acceptable salt thereof, irinotecan, an irinotecan derivative or a pharmaceutically acceptable salt thereof, and gemcitabine, a gemcitabine derivative or a pharmaceutically acceptable salt thereof, more preferably wherein said active pharmaceutical ingredient is selected from the group consisting of doxorubicin or a pharmaceutically acceptable salt thereof, gemcitabine or a pharmaceutically acceptable salt thereof, and irinotecan or a pharmaceutically acceptable salt thereof, and wherein said active pharmaceutical ingredient is comprised in the intraliposomal buffer, more preferably
   wherein the molar ratio between doxorubicin, said doxorubicin derivative or said pharmaceutically acceptable salt thereof and the lipids comprised in said bilayer is from 0.06 up to 0.10, preferably from 0.07 up to 0.09,
   wherein the molar ratio between irinotecan, said irinotecan derivative or said pharmaceutically acceptable salt thereof and the lipids comprised in said bilayer is at least 0.18, preferably at least 0.20,
   wherein the molar ratio between gemcitabine, said gemcitabine derivative or said pharmaceutically acceptable salt thereof and the lipids comprised in said bilayer is at least 0.12, preferably at least 0.15, and/or
wherein said intraliposomal buffer has a pH from 5 up to 8, preferably from 6 up to 8; and/or
wherein said bilayer does not comprise cholesterol or a derivative thereof; and/or
wherein said 1,2-dipalmitoyl-sn-glycero-3-phosphodiglycerol (DPPG₂) is represented by Formula (IV).

In a preferred embodiment is provided a thermosensitive liposome according to the invention wherein the bilayer comprised in said thermosensitive liposome does not comprise cholesterol. In the context of this application, a bilayer that does not comprise cholesterol means a bilayer that does not comprise a detectable amount of cholesterol or a derivative thereof. In this context, a derivative of cholesterol preferably means a cholesteryl ester.

Amphiphilic molecules comprised in a bilayer comprised in a thermosensitive liposome according to the invention have the tendency to form lamellar or micellar structures. An amphiphilic molecule with a tendency to form micellar structures thermodynamically favors the formation of micelles or micellar structures in suspension. An amphiphilic molecule with a tendency to form lamellar structures thermodynamically favors the formation of lamellar structures or liposomes in suspension. In the context of this application, an amphiphilic molecule with a high tendency to form micellar structures is called a surfactant. For example, most lysolipids and DSPE-PEG2000 are considered to be surfactants in the context of this application.

Without being bound to this theory, since a thermosensitive liposome according to the invention is a lamellar structure, the formation of said thermosensitive liposome is thermodynamically disfavored, i.e. said thermosensitive liposome is destabilized, by a high concentration of surfactants.

In a preferred embodiment is provided a thermosensitive liposome according to the invention wherein the concentration of surfactants is lower than 5 mol %, lower than 4 mol %, lower than 3 mol %, lower than 2 mol %, lower than 1 mol %, lower than 1 mol %, lower than 0.9 mol %, lower than 0.8 mol %, lower than 0.7 mol %, lower than 0.6 mol %, lower than 0.5 mol %, lower than 0.4 mol %, lower than 0.3 mol %, lower than 0.2 mol %, or lower than 0.1 mol %.

### Preferred Thermosensitive Liposomes

In a preferred embodiment is provided a thermosensitive liposome according to the invention wherein said active pharmaceutical ingredient is doxorubicin, a doxorubicin derivative or a pharmaceutically acceptable salt thereof, and wherein the molar ratio between doxorubicin, said doxorubicin derivative or said pharmaceutically acceptable salt thereof and the lipids comprised in said bilayer is from 0.06 up to 0.10, preferably from 0.07 up to 0.09,
wherein said thermosensitive liposome has a diameter from 100 nanometers up to 150 nanometers; and
wherein said intraliposomal buffer has a pH from 5 up to 8, preferably from 6 up to 8; and
preferably wherein said bilayer does not comprise cholesterol or a derivative thereof; and
wherein said bilayer comprises a 1,2-dipalmitoyl-sn-glycero-3-phosphocholine (DPPC) and 1,2-distearoyl-sn-glycero-3-phosphocholine (DSPC),
   wherein the molar concentration of 1,2-dipalmitoyl-sn-glycero-3-phosphocholine (DPPC) in said bilayer is from 0.45 up to 0.65, preferably from 0.45 up to 0.55, and
   wherein the molar concentration of 1,2-distearoyl-sn-glycero-3-phosphocholine (DSPC) in said bilayer is from 0.15 up to 0.25, and
   wherein the molar concentration of 1,2-dipalmitoyl-sn-glycero-3-phosphodiglycerol (DPPG₂) in said bilayer is from 0.15 up to 0.35, preferably from 0.25 up to 0.35; and
preferably wherein said 1,2-dipalmitoyl-sn-glycero-3-phosphodiglycerol (DPPG₂) is represented by Formula (IV).

Preferably, a thermosensitive liposome according to the embodiment above is comprised in a suitable drug delivery system, more preferably for use in the treatment of cancer.

In a preferred embodiment is provided a thermosensitive liposome according to the invention wherein said active pharmaceutical ingredient is irinotecan, an irinotecan derivative or a pharmaceutically acceptable salt thereof, and wherein the molar ratio between irinotecan, said irinotecan derivative or said pharmaceutically acceptable salt thereof and the lipids comprised in said bilayer is at least 0.18, preferably at least 0.20,
wherein said thermosensitive liposome has a diameter from 100 nanometers up to 150 nanometers; and
wherein said intraliposomal buffer has a pH from 5 up to 8, preferably from 6 up to 8; and
preferably wherein said bilayer does not comprise cholesterol or a derivative thereof; and
wherein said bilayer comprises a 1,2-dipalmitoyl-sn-glycero-3-phosphocholine (DPPC) and 1,2-distearoyl-sn-glycero-3-phosphocholine (DSPC),
   wherein the molar concentration of 1,2-dipalmitoyl-sn-glycero-3-phosphocholine (DPPC) in said bilayer is from 0.45 up to 0.65, preferably from 0.45 up to 0.55, and
   wherein the molar concentration of 1,2-distearoyl-sn-glycero-3-phosphocholine (DSPC) in said bilayer is from 0.15 up to 0.25, and
   wherein the molar concentration of 1,2-dipalmitoyl-sn-glycero-3-phosphodiglycerol (DPPG₂) in said bilayer is from 0.15 up to 0.35, preferably from 0.25 up to 0.35; and
preferably wherein said 1,2-dipalmitoyl-sn-glycero-3-phosphodiglycerol (DPPG₂) is represented by Formula (IV).

Preferably, a thermosensitive liposome according to the embodiment above is comprised in a suitable drug delivery system, more preferably for use in the treatment of cancer.

In a preferred embodiment is provided a thermosensitive liposome according to the invention wherein said active pharmaceutical ingredient is gemcitabine, a gemcitabine derivative or a pharmaceutically acceptable salt thereof, and wherein the molar ratio between gemcitabine, said gemcitabine derivative or said pharmaceutically acceptable salt thereof and the lipids comprised in said bilayer is at least 0.12, preferably at least 0.15,
wherein said thermosensitive liposome has a diameter from 100 nanometers up to 150 nanometers; and
wherein said intraliposomal buffer has a pH from 5 up to 8, preferably from 6 up to 8; and
preferably wherein said bilayer does not comprise cholesterol or a derivative thereof; and
wherein said bilayer comprises a 1,2-dipalmitoyl-sn-glycero-3-phosphocholine (DPPC) and 1,2-distearoyl-sn-glycero-3-phosphocholine (DSPC),
   wherein the molar concentration of 1,2-dipalmitoyl-sn-glycero-3-phosphocholine (DPPC) in said bilayer is from 0.45 up to 0.65, preferably from 0.45 up to 0.55, and
   wherein the molar concentration of 1,2-distearoyl-sn-glycero-3-phosphocholine (DSPC) in said bilayer is from 0.15 up to 0.25, and
   wherein the molar concentration of 1,2-dipalmitoyl-sn-glycero-3-phosphodiglycerol (DPPG₂) in said bilayer is from 0.15 up to 0.35, preferably from 0.25 up to 0.35; and
preferably wherein said 1,2-dipalmitoyl-sn-glycero-3-phosphodiglycerol (DPPG₂) is represented by Formula (IV).

Preferably, a thermosensitive liposome according to the embodiment above is comprised in a suitable drug delivery system, more preferably for use in the treatment of cancer.

### Further Thermosensitive Liposomes

The invention further provides a thermosensitive liposome comprising a bilayer and an intraliposomal buffer, wherein the molar concentration of 1,2-dipalmitoyl-sn-glycero-3-phosphodiglycerol (DPPG₂) in said bilayer is at least 15 percent, wherein said thermosensitive liposome comprises an active pharmaceutical ingredient, wherein said intraliposomal buffer has a pH from 5 up to 8, preferably from 6 up to 8. Such thermosensitive liposomes are referred to in the current application as further liposomes according to or of the invention or further thermosensitive liposomes according to or of the invention.

All preferred embodiments and definitions described above in the context for thermosensitive liposomes according to the invention apply to further thermosensitive liposomes according to the invention.

In a preferred embodiment is provided a further thermosensitive liposome according to the invention which is also a thermosensitive liposome according to the invention.

In a preferred embodiment is provided a further thermosensitive liposome according to the invention, wherein the molar ratio between said active pharmaceutical ingredient and the lipids comprised in said bilayer is from 0.05 up to 0.3.

In a preferred embodiment is provided a further thermosensitive liposome according to the invention, wherein said active pharmaceutical ingredient is doxorubicin, a doxorubicin derivative, or a pharmaceutically acceptable salt thereof, preferably wherein the molar ratio between said doxorubicin, said doxorubicin derivative or said pharmaceutically acceptable salt thereof and the lipids comprised in said bilayer is from 0.06 up to 0.10, more preferably from 0.07 up to 0.09.

In a preferred embodiment is provided a further thermosensitive liposome according to the invention, wherein said active pharmaceutical ingredient is irinotecan, an irinotecan derivative, or a pharmaceutically acceptable salt thereof, preferably wherein the molar ratio between said irinotecan, said irinotecan derivative or said pharmaceutically acceptable salt thereof and the lipids comprised in said bilayer is at least 0.18, more preferably at least 0.20.

In a preferred embodiment is provided a further thermosensitive liposome according to the invention, wherein said active pharmaceutical ingredient is gemcitabine, a gemcitabine derivative, or a pharmaceutically acceptable salt thereof, preferably wherein the molar ratio between said gemcitabine, said gemcitabine derivative or said pharmaceutically acceptable salt thereof and the lipids comprised in said bilayer is at least 0.12, more preferably at least 0.15.

### Preparation of Liposomes

Liposomes comprising an active pharmaceutical ingredient, such as thermosensitive liposomes according to the invention or further thermosensitive liposomes according to the invention, may be prepared via a wide range of different techniques such as lipid film hydration, ethanol injection and the extrusion method, as known by the skilled person. In Example 1, the preparation of thermosensitive liposomes according to the invention is described.

Preferably a liposome may be prepared by a method comprising:
a) preparing an unloaded liposome, wherein said unloaded liposome comprises a bilayer with the same lipid composition as said liposome, and wherein said unloaded liposome does not comprise the active pharmaceutical ingredient comprised in said liposome or wherein the concentration of said active pharmaceutical ingredient in the intraliposomal buffer of said unloaded liposome is low, as defined below;
ac) preferably extruding said unloaded liposome to obtain a unilamellar liposome; and
b) loading said unloaded liposome in a loading buffer.

During the preparation according to the method above of a liposome comprising an active pharmaceutical ingredient in the intraliposomal buffer, such as the thermosensitive liposome according to the invention described in Example 1, a corresponding unloaded liposome with no or a low concentration of said active pharmaceutical ingredient in said intraliposomal buffer is prepared first (an unloaded liposome). Then, the concentration of said active pharmaceutical ingredient in said intraliposomal buffer is increased during loading. A low concentration preferably means lower than 0.1, or lower than 0.01, or lower than 0.001, or lower than 0.0001 times the concentration of said active pharmaceutical ingredient in the intraliposomal buffer of the final liposome. No active pharmaceutical ingredient preferably means no detectable amount of said active pharmaceutical ingredient. Herein, the concentration of said active pharmaceutical ingredient may be determined via high performance liquid chromatography (H PLC), as shown in Example 7.

Loading, as performed in step b) in the method above is the transfer of an active pharmaceutical ingredient from the extraliposomal buffer to the intraliposomal buffer, whereby said active pharmaceutical ingredient passes the bilayer. In this sense, loading (of an active pharmaceutical ingredient) can be envisioned as the reverse process of the release (of an active pharmaceutical ingredient). The extraliposomal buffer during loading is also called the loading buffer in the context of this application.

In an aspect of the invention is provided a method for preparing a liposome comprising an active pharmaceutical ingredient, wherein the method comprises steps a) and b) as described above and, wherein said loading buffer has a salt concentration of at least 66 mM and osmolarity of at least 250 mOsmol/kg. More preferably, said loading buffer has a salt concentration of at least 66 mM and osmolarity from 250 mOsmol/kg up to 350 mOsmol/kg. Preferably, said liposome in said preferred method is a thermosensitive liposome according to the invention or a further thermosensitive liposome according to the invention.

In Example 6, it is shown that thermosensitive liposomes according to the invention may be prepared via the preferred method described above, using a loading buffer with a salt concentration of 66 mM and osmolarity of 294 mOsmol/kg or 300 mOsmol/kg. Specifically, it has been shown in Example 6 that loading buffers with high salt concentrations and osmolarities lead to shorter loading times (20 minutes for buffer A with an osmolarity of 300 mOsmol/kg without trehalose, 90 minutes for buffer C with an osmolarity of 294 mOsmol/kg and 8.9% (wt/v) trehalose).

In a further aspect of the invention is provided a method for preparing a liposome comprising an active pharmaceutical ingredient, wherein the method comprises steps a) and b) as described above, wherein said method comprises a buffer exchange, wherein said method comprises the use of a loading buffer and the use of a storage buffer, and wherein said storage buffer has a salt concentration lower than 100 mM and an osmolarity higher than 300 mOsmol/kg. More preferably, said storage buffer has a salt concentration lower than 100 mM and an osmolarity from 300 mOsmol/kg up to 450 mOsmol/kg.

The aqueous solution contacting the bilayer comprised in a liposome according to the invention is the extraliposomal buffer. In the context of (pharmaceutical) compositions, said extraliposomal buffer is called a storage buffer if said thermosensitive liposome is dispersed in said extraliposomal buffer.

Example 9 shows that a method employing a buffer exchange to a storage buffer with a low saline concentration leads to a lower leakage of the active pharmaceutical ingredient comprised therein, and to a higher dispersion stability.

A method according to the above aspects of the invention is called a method according to the invention in the context of the application. It must be noted that the definitions and embodiments provided above in the context of thermosensitive liposomes may be applied mutatis mutandis to liposomes in general as mentioned in the context of a method according to the invention. For example, the diameter of a thermosensitive liposome is clearly defined in the same way as the diameter of a liposome for which a method of preparation is described herein.

It is clear to the skilled person that a composition according to the invention or a further composition according to the invention may also be prepared via a method according to the invention, or via a method comprising a method according to the invention.

Any preference or embodiment relating to a thermosensitive liposome according to the invention, or a composition comprising a thermosensitive liposome according to the invention, may be combined with a method according to the invention, meaning that these preferences and embodiments also disclose corresponding methods according to the invention for preparing such thermosensitive liposomes or compositions.

In the context of a method according to the invention, it is clear that a concentration relative to the lipids comprised in a bilayer, may either refer to the lipids comprised in the bilayer of said unloaded liposome or the bilayer of said liposome, as the lipid compositions of these bilayers is the same.

In a preferred embodiment is provided a method according to the invention, wherein the loading buffer has a salt concentration of at least 66 mM and an osmolarity from 250 mOsmol/kg up to 350 mOsmol/kg, and said storage buffer has a salt concentration lower than 100 mM and an osmolarity higher than 300 mOsmol/kg. Most preferably, said loading buffer has a salt concentration from 66 mM up to 120 mM and an osmolarity from 250 mOsmol/kg up to 350 mOsmol/kg, and said storage buffer has a salt concentration lower than 100 mM and an osmolarity from 390 mOsmol/kg.

In a preferred embodiment is provided a method according to the invention, wherein said liposome comprises 1,2-dipalmitoyl-sn-glycero-3-phosphodiglycerol (DPPG₂), preferably wherein the molar concentration of DPPG₂ in the bilayer of said liposome is from 0.10 up to 0.95, from 0.10 up to 0.90, from 0.10 up to 0.85, from 0.10 up to 0.80, from 0.10 up to 0.75, from 0.10 up to 0.70, from 0.10 up to 0.65, from 0.10 up to 0.60, from 0.10 up to 0.55, from 0.10 up to 0.50, from 0.10 up to 0.45, from 0.10 up to 0.40, from 0.15 up to 0.35, or from 0.20 up to 0.30.

In a more preferred embodiment is provided a method according to the invention, wherein said liposome is a thermosensitive liposome. In an even more preferred embodiment is provided a method according to the invention, wherein said liposome is a thermosensitive liposome according to the invention or a further thermosensitive liposome according to the invention. In a most preferred embodiment is provided a method according to the invention, wherein said liposome is a thermosensitive liposome according to the invention.

In a preferred embodiment is provided a method according to the invention, wherein the molar concentration of 1,2-dipalmitoyl-sn-glycero-3-phosphocholine (DPPC) in said bilayer comprised in said unloaded liposome is from 0.45 up to 0.65, preferably from 0.45 up to 0.55; and/or wherein the molar concentration of 1,2-distearoyl-sn-glycero-3-phosphocholine (DSPC) in said bilayer comprised in said unloaded liposome is from 0.15 up to 0.25; and/or wherein the molar concentration of 1,2-dipalmitoyl-sn-glycero-3-phosphodiglycerol (DPPG₂) in said bilayer comprised in said unloaded liposome is from 0.15 up to 0.35, preferably from 0.25 up to 0.35.

In a preferred embodiment is provided a method according to the invention, wherein said unloaded liposome has a diameter from 100 nanometers up to 200 nanometers, preferably from 100 nanometers up to 150 nanometers.

In a preferred embodiment is provided a method according to the invention,
wherein said active pharmaceutical ingredient is doxorubicin, a doxorubicin derivative or a pharmaceutically acceptable salt thereof, and wherein the molar ratio between doxorubicin, said doxorubicin derivative or said pharmaceutically acceptable salt thereof and the lipids comprised in said bilayer is from 0.06 up to 0.10, preferably from 0.07 up to 0.09; or
wherein said active pharmaceutical ingredient is irinotecan, an irinotecan derivative or a pharmaceutically acceptable salt thereof, and wherein the molar ratio between irinotecan, said irinotecan derivative or said pharmaceutically acceptable salt thereof and the lipids comprised in said bilayer is at least 0.18, preferably at least 0.20; or
wherein said active pharmaceutical ingredient is gemcitabine, a gemcitabine derivative or a pharmaceutically acceptable salt thereof, and wherein the molar ratio between gemcitabine, said gemcitabine derivative or said pharmaceutically acceptable salt thereof and the lipids comprised in said bilayer is at least 0.12, preferably at least 0.15.

Preferably, a method according to the invention does not comprise adding cholesterol or a derivative thereof.

In a preferred embodiment is provided a method according to the invention, wherein step a) comprised in said method comprises:
aa) preparing a solution of lipids in an organic solvent with the same molar ratio as the lipids in the bilayer of said liposome; and
ab) mixing said solution of lipids with an aqueous solution,
   wherein said aqueous solution has essentially the same composition as the intraliposomal buffer comprised in said liposome, without the presence of said active pharmaceutical ingredient, preferably wherein said aqueous solution has a pH from 5 up to 8, preferably from 6 up to 8, and
   wherein said mixing is preferably performed via organic solvent injection, and
   wherein said mixing leads to the formation of an unloaded liposome; and
ac) preferably extruding said unloaded liposome to obtain a unilamellar liposome.

In a preferred embodiment is provided a method according to the invention wherein step a) comprised in said method comprises steps aa), ab) and ac), as defined above, and wherein said organic solvent used in step aa) is selected from the group of organic solvents capable to dissolve lipids, preferably wherein said organic solvent is selected from the group consisting of chloroform/methanol 9:1 (vol/vol), chloroform, methanol, isopropanol, ethanol and mixtures thereof.

In a preferred embodiment is provided a method according to the invention wherein step a) comprised in said method comprises steps aa), ab) and ac), as defined above, and wherein said aqueous solution used in step ab) comprises a buffer selected from the group consisting of a (NH₄)₂SO₄ buffer, a (NH₄)₂HPO₄ buffer, a phosphate buffer and HBS buffer.

In a preferred embodiment is provided a method according to the invention wherein step a) comprised in said method comprises steps aa), ab) and ac), as defined above, and wherein said extrusion is performed at temperatures where the lipids are in a liquid phase state.

Said loading of an unloaded liposome in step b) of a method according to the invention leads to the transfer of said active pharmaceutical ingredient to the intraliposomal buffer, and is described in more detail below.

Preferably said loading is carried out at a temperature from 35° C. to 39° C., more preferably from 36° C. to 38° C., most preferably from 37° C. to 38° C.

A method according to the invention preferably further comprises filtration steps.

The preferred preparation method above is exemplified in FIG. 2, wherein an ethanol injection is used in step (ab). It should be noted that FIG. 2 mentions two buffer exchanges. The first one involves the exchange of the extraliposomal buffer at the end of step (ab) for the loading buffer. The second one involves the exchange of the loading buffer for the storage buffer.

Preferably, a method according to the invention comprises a buffer exchange. A buffer exchange is defined as completely or partially substituting the extraliposomal buffer by a new extraliposomal buffer. Herein, it is understood that substituting may be adjusting the concentration of component in said buffer, adding a component to said buffer, and/or removing a component from said buffer. A preferred method for buffer exchange is selected from the group consisting of tangential flow filtration (TFF), chromatography and centrifugation, preferably tangential flow filtration.

Passive loading is loading driven by a gradient in the concentration of said active pharmaceutical ingredient across said bilayer. For a thermosensitive liposome according to the invention, at a temperature above the transition temperature of said thermosensitive liposome, a high ratio between the concentration of said active pharmaceutical ingredient in the extraliposomal buffer and the concentration of said active pharmaceutical ingredient in the intraliposomal buffer leads to a transfer across said bilayer, which is in the liquid phase. A high ratio preferably means at least 10, or at least 100, or at least 1000, or at least 10000. Preferably, at the start of passive loading there is no detectable amount of said active pharmaceutical ingredient in said intraliposomal buffer. Herein, the concentration of said active pharmaceutical ingredient is determined via HPLC. After a desired amount of active pharmaceutical ingredient is transferred to the intraliposomal buffer, the temperature is decreased and said bilayer reverts to the gel phase. Since the permeability of the bilayer is lower in the gel phase, the active pharmaceutical ingredient in the intraliposomal will not easily transfer to the extraliposomal buffer, even if the concentration of the active pharmaceutical ingredient in the extraliposomal is lowered.

Active loading is loading driven by a gradient of an ion across said bilayer. This may be an ammonium gradient, a proton gradient (i.e. a pH gradient) or an EDTA salt gradient for example. For example, doxorubicin, irinotecan and gemcitabine, all of which are water-soluble active pharmaceutical ingredients, may be transferred to the intraliposomal buffer in a thermosensitive liposome according to the invention via active loading. This is described in detail in Example 1.

Preferably, a method according to the invention comprises d) storing said loaded liposome in a storage buffer from 1 up to 5 weeks at a temperature around 5° C., from 1 up to 10 weeks at a temperature around 5° C., from 1 up to 15 weeks at a temperature around 5° C., from 1 up to 20 weeks at a temperature around 5° C., or from 1 up to 20 weeks at a temperature around 5° C., or from 1 up to 16 months at a temperature around −20° C., or from 12 up to 16 months at a temperature around −20° C. More preferably, said loaded liposome is stable upon said storage, as defined below.

Preferably, a method according to the invention comprises adding at least one excipient that may further aid in enhancing the delivery of said liposome and/or of a composition comprising said composition, to a tissue and/or cell and/or into a tissue and/or cell.

In a preferred embodiment is provided a method for preparing a liposome, preferably a thermosensitive liposome, more preferably a thermosensitive liposome according to the invention or a further thermosensitive liposome according to the invention, most preferably a thermosensitive liposome according to the invention, comprising:
a) preparing an unloaded liposome, wherein said unloaded liposome comprises a bilayer with the same lipid composition as said liposome, and wherein said unloaded liposome does not comprise the active pharmaceutical ingredient comprised in said liposome, wherein said a) preparing of said unloaded liposome comprises:
   aa) preparing a solution of lipids in an organic solvent with the same molar ratio as the lipids in the bilayer of said liposome, preferably wherein said organic solvent is chloroform/methanol 9:1 (vol/vol), chloroform or ethanol; and
   ab) mixing said solution of lipids with an aqueous solution,
      wherein said aqueous solution has essentially the same composition as the intraliposomal buffer comprised in said liposome, without the presence of said active pharmaceutical ingredient, preferably wherein said aqueous solution has a pH from 5 up to 8, preferably from 6 up to 8, and
      preferably wherein said aqueous solution comprises a buffer selected from the group consisting of a (NH₄)₂SO₄ buffer, a (NH₄)₂HPO₄ buffer, a phosphate and an HBS buffer, and
      wherein said mixing is preferably performed via organic solvent injection, and
      wherein said mixing leads to the formation of an unloaded liposome; and
   ac) preferably extruding said unloaded liposome,
b) loading said unloaded liposome with active pharmaceutical ingredient by active loading using a loading buffer to form a loaded liposome, wherein said loading buffer has a salt concentration from 66 mM up to 120 mM and an osmolarity from 250 mOsmol/kg up to 350 mOsmol/kg, wherein said loading is preferably carried out at a temperature from 35° C. to 39° C., more preferably from 36° C. to 38° C., most preferably from 37° C. to 38° C.;
c) exchanging said loading buffer by a storage buffer, wherein said storage buffer has a salt concentration lower than 100 mM and an osmolarity higher than 300 mOsmol/kg,
d) preferably storing said loaded liposome in said storage buffer from 1 up to 20 weeks at a temperature around 5° C., or from 1 up to 20 weeks at a temperature around 5° C., or from 1 up to 16 months at a temperature around −20° C., or from 12 up to 16 months at a temperature around −20° C.

In a preferred embodiment is provided a method according to the previous embodiment, wherein the liposome obtained at the end of step c) is stable upon said storing in step d), wherein stable upon storing or storage is defined below.

In a more preferred embodiment is provided a method according to the previous embodiment, preferably wherein said storing in step d) is from 12 up to 16 months at a temperature around −20° C., wherein the liposome obtained at the end of step c) is stable upon storage, wherein stable upon storage means that:
the concentration of the active pharmaceutical ingredient comprised in said liposome obtained at the end of step c) does not change by more than 15% upon storage; and/or
the diameter of said liposome obtained at the end of step c) does not change by more than 30% upon storage; and/or
the concentration in the bilayer of lysolipids comprised in said liposome obtained at the end of step c) does not increase above 5% during storage; and/or
the polydispersity of said thermosensitive liposome or the polydispersity of said liposome obtained at the end of step c) does not increase above 0.5, during storage, and/or
if said liposome obtained at the end of step c) is characterized by a high selective delivery upon heat treatment, then said liposome is still characterized by a high selective delivery upon heat treatment after storage; and/or
if said liposome obtained at the end of step c) is a suitable drug delivery system, then said liposome is still a suitable drug delivery system after storage.

### Composition

In an aspect of the invention is provided a composition comprising a thermosensitive liposome according to the invention, preferably wherein said composition comprises at least one excipient that may further aid in enhancing the targeting or delivery of said composition or said thermosensitive liposome or said active pharmaceutical ingredient to a tissue or cell or into a tissue or cell. A preferred tissue or cell is a tumor or tumor cell. Compositions as described here are herein referred to as compositions according to or of the invention. A composition according to the invention can comprise one or more than one thermosensitive liposome according to the invention. In the context of this invention, an excipient can be a distinct molecule, but it can also be a conjugated moiety.

In a preferred embodiment, said composition is for use as a medicament, preferably for use in treating, ameliorating, delaying, curing and/or preventing cancer. Said composition is therefore a pharmaceutical composition. A pharmaceutical composition usually comprises a pharmaceutically accepted carrier, diluent and/or excipient. In a preferred embodiment, a composition of the current invention comprises a thermosensitive liposome as defined herein and further comprises a pharmaceutically acceptable formulation, filler, preservative, solubilizer, carrier, diluent, excipient, salt, adjuvant and/or solvent. Such pharmaceutically acceptable carrier, filler, preservative, solubilizer, diluent, salt, adjuvant, solvent and/or excipient may for instance be found in Remington: The Science and Practice of Pharmacy, 20th Edition. Baltimore, MD: Lippincott Williams & Wilkins, 2000.

A thermosensitive liposome according to the invention may possess at least one ionizable group. An ionizable group may be a base or acid, and may be charged or neutral. An ionizable group may be present as ion pair with an appropriate counter ion that carries opposite charge(s). Examples of cationic counter ions are sodium, potassium, cesium, Tris, lithium, calcium, magnesium, trialkylammonium, triethylammonium, and tetraalkylammonium. Examples of anionic counter ions are chloride, bromide, iodide, lactate, mesylate, besylate, triflate, acetate, trifluoroacetate, dichloroacetate, tartrate, lactate, and citrate.

A pharmaceutical composition according to the invention may comprise an aid in enhancing the stability, solubility, absorption, bioavailability, activity, pharmacokinetics, pharmacodynamics, cellular uptake, and intracellular trafficking of said active pharmaceutical ingredient.

A composition according to the invention may be administered in an effective concentration at set times to an animal, preferably a mammal. Administration may be via topical, systemic and/or parenteral routes, for example intravenous, subcutaneous, intraperitoneal, intrathecal, intramuscular, ocular, nasal, urogenital, intradermal, dermal, enteral, intravitreal, intracavernous, intracerebral, intrathecal, epidural or oral route.

Compounds that are comprised in a composition according to the invention can also be provided separately, for example to allow sequential administration of the active components of the composition according to the invention. In such a case, the composition according to the invention is a combination of compounds comprising at least a thermosensitive liposome according to the invention, and at least one excipient.

As defined above, the aqueous solution contacting the bilayer comprised in a thermosensitive liposome according to the invention is the extraliposomal buffer. In the context of (pharmaceutical) compositions, said extraliposomal buffer is called a storage buffer if said thermosensitive liposome is dispersed in said extraliposomal buffer.

In a preferred embodiment is provided a composition according to the invention comprising a storage buffer, wherein said storage buffer has a saline concentration lower than 120 mM, or lower than 110 mM, or lower than 100 mM, or lower than 90 mM, or lower than 80 mM, or lower than 70 mM, or lower than 60 mM, or lower than 50 mM, or lower than 40 mM, or lower than 30 mM, or lower than 20 mM, or lower than 10 mM.

Example 9 shows that a composition according to the invention characterized by a low saline concentration leads to a lower leakage of the active pharmaceutical ingredient comprised therein, and to a higher dispersion stability.

In another preferred embodiment is provided a composition according to the invention comprising a storage buffer, wherein the osmolarity of said storage buffer is higher than 150 mOsmol/kg, or higher than 200 mOsmol/kg, or higher than 250 mOsmol/kg, or higher than 300 mOsmol/kg, or higher than 350 mOsmol/kg, or higher than 400 mOsmol/kg. Preferably, the osmolarity of said storage buffer is lower than 450 mOsmol/kg, more preferably lower than 410 mOsmol/kg. In a most preferred embodiment is provided a composition according to the invention comprising a storage buffer, wherein the osmolarity of said storage buffer is from 200 mOsmol/kg up to 450 mOsmol/kg, or wherein the osmolarity of said storage buffer is from 300 mOsmol/kg up to 450 mOsmol/kg, or wherein the osmolarity of said storage buffer is from 200 mOsmol/kg up to 410 mOsmol/kg, or wherein the osmolarity of said storage buffer is from 300 mOsmol/kg up to 410 mOsmol/kg.

In another preferred embodiment is provided a composition according to the invention comprising a storage buffer, wherein said storage buffer comprises a cryoprotectant. Preferably, said cryoprotectant is trehalose or sucrose. Most preferably, said cryoprotectant is trehalose.

The inclusion of a cryoprotectant in a composition according to the invention allows for storage of said composition at low temperature in order to prevent degradation of the active pharmaceutical ingredient comprised in the thermosensitive liposome comprised in said composition. In a preferred embodiment is provided a composition according to the invention wherein said composition is still a suitable drug delivery system, as defined above, after storage below 0° C., or below −5° C., or below −10° C., or below −15° C., or below −20° C. for at least 30 days.

Example 9 shows that a composition according to the invention characterized by a high osmolarity and the presence of trehalose as a cryoprotectant is able to prevent active pharmaceutical ingredient degradation.

In a preferred embodiment is provided a composition according to the invention, wherein the thermosensitive liposome comprised in said composition is dispersed in a storage buffer, wherein the saline concentration of said storage buffer is lower than 100 mM and/or the osmolarity of said storage buffer is higher than 300 mOsmol/kg. Preferably, a composition according to this embodiment comprises a cryoprotectant.

In a preferred embodiment is provided a composition according to the invention comprising multiple thermosensitive liposomes according to the invention dispersed in a storage buffer, wherein the concentration of said thermosensitive liposomes in said storage buffer is from 10 mmol/l up to 50 mmol/l, or from 15 mmol/l up to 50 mmol/l, or from 20 mmol/l up to 50 mmol/l, or from 25 mmol/l up to 50 mmol/l, or from 30 mmol/l up to 50 mmol/l, or from 35 mmol/l up to mmol/l, or from 37.5 mmol/l up to 42.5 mmol/l. Preferably, the saline concentration of said storage buffer is lower than 100 mM and/or the osmolarity of said storage buffer is higher than 300 mOsmol/kg.

In another preferred embodiment is provided a composition according to the invention wherein the concentration of thermosensitive liposomes according to the invention is from 35 mmol/l up to 45 mmol/l, wherein the polydispersity index (PDI) of said thermosensitive liposomes is lower than 0.5, or lower than 0.4, or lower than 0.3, or lower than 0.2, or lower than 0.1, or lower than 0.09, or lower than 0.08, or lower than 0.07, or lower than 0.06, or lower than 0.05. Preferably, the saline concentration of said storage buffer is lower than 100 mM and/or the osmolarity of said storage buffer is higher than 300 mOsmol/kg.

In another preferred embodiment is provided a composition according to the invention wherein the diameter of the thermosensitive liposomes according to the invention comprised in said composition is from 100 nm up to 150 nm, wherein the polydispersity index (PDI) of said thermosensitive liposomes is lower than 0.5, or lower than 0.4, or lower than 0.3, or lower than 0.2, or lower than 0.1, or lower than 0.09, or lower than 0.08, or lower than 0.07, or lower than 0.06, or lower than 0.05; more preferably wherein the polydispersity of said thermosensitive liposomes is lower than 0.1. Preferably, the saline concentration of said storage buffer is lower than 100 mM and/or the osmolarity of said storage buffer is higher than 300 mOsmol/kg.

In a preferred embodiment is provided a composition according to the invention comprising a storage buffer, wherein the ratio between the concentration of said active pharmaceutical ingredient in said storage buffer and the concentration of said active pharmaceutical ingredient in the intraliposomal buffer comprised in the thermosensitive liposomes comprised in said composition is lower than 0.1, or lower than 0.09, or lower than 0.08, or lower than 0.07, or lower than 0.06, or lower than 0.05, or lower than 0.04, or lower than 0.03, or lower than 0.02, or lower than 0.01, or lower than 0.009, or lower than 0.008, or lower than 0.007, or lower than 0.006, or lower than 0.005, or lower than 0.004, or lower than 0.003, or lower than 0.002, or lower than 0.001.

In a preferred embodiment is provided a composition according to the invention,
wherein the concentration of thermosensitive liposomes according to the invention is from 10 mmol/l up to 50 mmol/l, preferably from 35 mmol/l up to 45 mmol/l; and
wherein said composition comprises a storage buffer, wherein the saline concentration of said storage buffer is lower than 100 mM and the osmolarity of said storage buffer is higher than 300 mOsmol/kg; and
wherein the active pharmaceutical ingredients comprised in said thermosensitive liposomes are doxorubicin, a doxorubicin derivative or a pharmaceutically acceptable salt thereof, and wherein the molar ratio between doxorubicin, said doxorubicin derivative or said pharmaceutically acceptable salt thereof and the lipids comprised in the bilayers comprised in said thermosensitive liposomes is from 0.06 up to 0.10, preferably from 0.07 up to 0.09; and
wherein said thermosensitive liposomes have a diameter from 100 nanometers up to 150 nanometers; and
wherein the intraliposomal buffers comprised in said thermosensitive liposomes have a pH from 5 up to 8, preferably from 6 up to 8; and
preferably wherein the bilayers comprised in said thermosensitive liposomes do not comprise cholesterol or a derivative thereof; and
wherein the bilayers comprised in said thermosensitive liposomes comprise 1,2-dipalmitoyl-sn-glycero-3-phosphocholine (DPPC) and 1,2-distearoyl-sn-glycero-3-phosphocholine (DSPC),
   wherein the molar concentration of 1,2-dipalmitoyl-sn-glycero-3-phosphocholine (DPPC) in said bilayers is from 0.45 up to 0.65, preferably from 0.45 up to 0.55, and
   wherein the molar concentration of 1,2-distearoyl-sn-glycero-3-phosphocholine (DSPC) in said bilayers is from 0.15 up to 0.25, and
   wherein the molar concentration of 1,2-dipalmitoyl-sn-glycero-3-phosphodiglycerol (DPPG₂) in said bilayers is from 0.15 up to 0.35, preferably from 0.25 up to 0.35.

In a more preferred embodiment is provided a composition according to the preferred embodiment above, wherein the polydispersity index (PDI) of said thermosensitive liposomes is lower than 0.5, or lower than 0.4, or lower than 0.3, or lower than 0.2, or lower than 0.1, or lower than 0.09, or lower than 0.08, or lower than 0.07, or lower than 0.06, or lower than 0.05.

In Example 10, it is shown that a composition according to the embodiment above is a suitable drug delivery system.

Specifically, such a composition is shown to be stable upon storage for an extended period of time (16 months) when stored at −20° C.±5° C. with negligible doxorubicin leakage and change in thermosensitive liposome diameter. The results further indicated that the composition was stable for 11 weeks when stored at 5° C.±3° C. No signs of phospholipid decomposition were visible during the 12 months of storage. Moreover, the composition was stable for six freezing and thawing cycles. Furthermore, the accumulation of doxorubicin in tumors was shown in vivo via a biodistribution study in Brown Norway rats with a s.c. BN175 sarcoma on a hind limb. Animals treated with the composition showed a doxorubicin concentration in the heated tumor which was 15.8 times higher than the concentration in the non-heated tissue. Treatment of rats with the composition showed significantly improved tumor growth delay versus animals treated with saline (FIG. 13). Furthermore, compared to the non-liposomal doxorubicin treatment at equivalent dose, the composition also yielded significant tumor growth delay and prolonged survival (FIG. 13).

In a preferred embodiment is provided a composition according to the invention,
wherein the concentration of thermosensitive liposomes according to the invention is from 10 mmol/l up to 50 mmol/l, preferably from 35 mmol/l up to 45 mmol/l; and
wherein said composition comprises a storage buffer, wherein the saline concentration of said storage buffer is lower than 100 mM and the osmolarity of said storage buffer is higher than 300 mOsmol/kg; and
wherein the active pharmaceutical ingredients comprised in said thermosensitive liposomes are irinotecan, a derivative of irinotecan or a pharmaceutically acceptable salt thereof, and wherein the molar ratio between irinotecan, said derivative of irinotecan or said pharmaceutically acceptable salt thereof and the lipids comprised in the bilayers comprised in said thermosensitive liposomes is at least 0.18, preferably at least 0.20; and
wherein said thermosensitive liposomes have a diameter from 100 nanometers up to 150 nanometers; and
wherein the intraliposomal buffers comprised in said thermosensitive liposomes have a pH from 5 up to 8, preferably from 6 up to 8; and
preferably wherein the bilayers comprised in said thermosensitive liposomes do not comprise cholesterol or a derivative thereof; and
wherein the bilayers comprised in said thermosensitive liposomes comprise 1,2-dipalmitoyl-sn-glycero-3-phosphocholine (DPPC) and 1,2-distearoyl-sn-glycero-3-phosphocholine (DSPC),
   wherein the molar concentration of 1,2-dipalmitoyl-sn-glycero-3-phosphocholine (DPPC) in said bilayers is from 0.45 up to 0.65, preferably from 0.45 up to 0.55, and
   wherein the molar concentration of 1,2-distearoyl-sn-glycero-3-phosphocholine (DSPC) in said bilayers is from 0.15 up to 0.25, and
   wherein the molar concentration of 1,2-dipalmitoyl-sn-glycero-3-phosphodiglycerol (DPPG₂) in said bilayers is from 0.15 up to 0.35, preferably from 0.25 up to 0.35.

In a more preferred embodiment is provided a composition according to the preferred embodiment above, wherein the polydispersity index (PDI) of said thermosensitive liposomes is lower than 0.5, or lower than 0.4, or lower than 0.3, or lower than 0.2, or lower than 0.1, or lower than 0.09, or lower than 0.08, or lower than 0.07, or lower than 0.06, or lower than 0.05.

In a preferred embodiment is provided a composition according to the invention,
wherein the concentration of thermosensitive liposomes according to the invention is from 10 mmol/l up to 50 mmol/l, preferably from 35 mmol/l up to 45 mmol/l; and
wherein said composition comprises a storage buffer, wherein the saline concentration of said storage buffer is lower than 100 mM and the osmolarity of said storage buffer is higher than 300 mOsmol/kg; and
wherein the active pharmaceutical ingredients comprised in said thermosensitive liposomes are gemcitabine, a gemcitabine derivative or a pharmaceutically acceptable salt thereof, and wherein the molar ratio between gemcitabine, said gemcitabine derivative or said pharmaceutically acceptable salt thereof and the lipids comprised in the bilayers comprised in said thermosensitive liposomes is at least 0.12, preferably at least 0.15; and
wherein said thermosensitive liposomes have a diameter from 100 nanometers up to 150 nanometers; and
wherein the intraliposomal buffers comprised in said thermosensitive liposomes have a pH from 5 up to 8, preferably from 6 up to 8; and
preferably wherein the bilayers comprised in said thermosensitive liposomes do not comprise cholesterol or a derivative thereof; and
wherein the bilayers comprised in said thermosensitive liposomes comprise 1,2-dipalmitoyl-sn-glycero-3-phosphocholine (DPPC) and 1,2-distearoyl-sn-glycero-3-phosphocholine (DSPC),
   wherein the molar concentration of 1,2-dipalmitoyl-sn-glycero-3-phosphocholine (DPPC) in said bilayers is from 0.45 up to 0.65, preferably from 0.45 up to 0.55, and
   wherein the molar concentration of 1,2-distearoyl-sn-glycero-3-phosphocholine (DSPC) in said bilayers is from 0.15 up to 0.25, and
   wherein the molar concentration of 1,2-dipalmitoyl-sn-glycero-3-phosphodiglycerol (DPPG₂) in said bilayers is from 0.15 up to 0.35, preferably from 0.25 up to 0.35.

In a more preferred embodiment is provided a composition according to the preferred embodiment above, wherein the polydispersity index (PDI) of said thermosensitive liposomes is lower than 0.5, or lower than 0.4, or lower than 0.3, or lower than 0.2, or lower than 0.1, or lower than 0.09, or lower than 0.08, or lower than 0.07, or lower than 0.06, or lower than 0.05.

### Further Composition

The invention further provides a composition comprising a thermosensitive liposome dispersed in a storage buffer, wherein the saline concentration of said storage buffer is lower than 100 mM and the osmolarity of said storage buffer is higher than 300 mOsmol/kg; wherein said thermosensitive liposome comprises a bilayer and an intraliposomal buffer; wherein the molar concentration of 1,2-dipalmitoyl-sn-glycero-3-phosphodiglycerol (DPPG₂) in said bilayer is at least 15 percent; and wherein said thermosensitive liposome comprises an active pharmaceutical ingredient. Such compositions are referred to in the current application as further compositions according to or of the invention.

All preferred embodiments and definitions described above in the context for compositions according to the invention or thermosensitive liposomes according to the invention apply to further compositions according to the invention.

In a preferred embodiment is provided a further composition according to the invention which is also a composition according to the invention.

In a preferred embodiment is provided a further composition according to the invention, wherein said intraliposomal buffer has a pH from 5 up to 8, preferably from 6 up to 8.

In a preferred embodiment is provided a further composition according to the invention, wherein said further composition comprises at least one excipient that may further aid in enhancing the delivery of said composition and/or said thermosensitive liposome to a tissue and/or cell and/or into a tissue and/or cell.

In a preferred embodiment is provided a further composition according to the invention, wherein said active pharmaceutical ingredient is doxorubicin, a doxorubicin derivative, or a pharmaceutically acceptable salt thereof, preferably wherein the molar ratio between said doxorubicin, said doxorubicin derivative or said pharmaceutically acceptable salt thereof and the lipids comprised in said bilayer is from 0.06 up to 0.10, more preferably from 0.07 up to 0.09.

In a preferred embodiment is provided a further composition according to the invention, wherein said active pharmaceutical ingredient is irinotecan, an irinotecan derivative, or a pharmaceutically acceptable salt thereof, preferably wherein the molar ratio between said irinotecan, said irinotecan derivative or said pharmaceutically acceptable salt thereof and the lipids comprised in said bilayer is at least 0.18, more preferably at least 0.20.

In a preferred embodiment is provided a further composition according to the invention, wherein said active pharmaceutical ingredient is gemcitabine, a gemcitabine derivative, or a pharmaceutically acceptable salt thereof, preferably wherein the molar ratio between said gemcitabine, said gemcitabine derivative or said pharmaceutically acceptable salt thereof and the lipids comprised in said bilayer is at least 0.12, more preferably at least 0.15.

### Drug Delivery System

A drug delivery system is a composition comprising an active pharmaceutical ingredient, wherein the other compounds comprised in said composition aid in enhancing the stability, solubility, absorption, bioavailability, activity, pharmacokinetics, pharmacodynamics, cellular uptake, and/or intracellular trafficking of said active pharmaceutical ingredient. Preferably, a thermosensitive liposome according to the invention, a further thermosensitive liposome according to the invention, a composition according to the invention or a further composition according to the invention is a drug delivery system.

In a preferred embodiment is provided a thermosensitive liposome according to the invention, a further thermosensitive liposome according to the invention, a composition according to the invention or a further composition according to the invention, wherein said thermosensitive liposome, said further thermosensitive liposome, said composition or said further composition has one or more of the following properties, each of which is defined below:
a low complement activation,
the absence of an anaphylaxis,
non-toxicity,
a long circulation half-time,
the absence of ABC,
a suitable clearance,
a suitable biodistribution,
a high selective delivery upon heat treatment
a high stability upon storage.

A thermosensitive liposome according to the invention, a further thermosensitive liposome according to the invention, a composition according to the invention or a further composition according to the invention characterized by all properties above is suitable for use as a drug delivery system, as explained below, and is called a suitable drug delivery system in the context of this application. In a preferred embodiment is provided a suitable drug delivery system.

Administration of a thermosensitive liposome according to the invention, a further thermosensitive liposome according to the invention, a composition according to the invention or a further composition according to the invention to an animal may result in adverse reactions such as activation of the complement system and anaphylaxis.

The complement system is part of the non-adaptive immune system, as known to the skilled person. Activation of the complement system, in which granulocytes, and mainly neutrophils, play a key role, may lead to complement activation-related pseudoallergy (CARPA). Evidently, the activation of the complement system by administration of a drug delivery system is unwanted. As explained in Example 2, complement activation associated with a thermosensitive liposome according to the invention or a composition according to the invention may be determined in vitro with C3a, Bb and SC5b-9 ELISA-kits on thermosensitive liposome incubated human plasma. Low complement activation means a complement activation at least times lower, or at least 9 times lower, or at least 8 times lower, or at least 7 times lower, or at least 6 times lower, or at least 5 times lower, or at least 4 times lower, or at least 3 times lower, or at least 2 times lower compared to a positive control (Zymosan), wherein said complement activation is determined as a C3a readout, a Bb readout or a SC5b-9 readout, following the protocol described in Example 2.

In a preferred embodiment is provided a thermosensitive liposome according to the invention, a further thermosensitive liposome according to the invention, a composition according to the invention or a further composition according to the invention, wherein said (further) thermosensitive liposome or (further) composition has a low complement activation after administration of said (further) thermosensitive liposome or (further) composition to an animal.

Anaphylaxis is a serious rapid onset allergic reaction, as known to the skilled person. Evidently, anaphylaxis caused by administration by a drug delivery system is unwanted. The absence of anaphylaxis, or anaphylactic reactions, is defined for a (further) thermosensitive liposome or a (further) composition as the absence of anaphylaxis after administration of said thermosensitive liposome or said composition, determined as outlined in Example 2.

In a preferred embodiment is provided a thermosensitive liposome according to the invention, a further thermosensitive liposome according to the invention, a composition according to the invention or a further composition according to the invention, wherein said (further) thermosensitive liposome or (further) composition does not lead to anaphylaxis after administration of said thermosensitive liposome or composition to an animal.

Preferably, a thermosensitive liposome according to the invention, a further thermosensitive liposome according to the invention, a composition according to the invention or a further composition according to the invention is defined as non-toxic if administration of said (further) thermosensitive liposome or said (further) composition to an animal is characterized by a low complement activation and the absence of anaphylaxis. In a preferred embodiment is provided a thermosensitive liposome according to the invention, a further thermosensitive liposome according to the invention, a composition according to the invention or a further composition according to the invention which is not toxic.

A drug delivery system is administered to an animal in order to deliver the active pharmaceutical ingredient comprised in said drug delivery system to a target being a cell comprised in said animal or a part thereof, or a tissue comprised in said animal or a part thereof, or an organ comprised in said animal or a part thereof. In the context of this application, said cell is preferably a tumor cell and said tissue or part thereof is preferably a tumor. Delivery of an active pharmaceutical ingredient by a drug delivery system means that the active pharmaceutical ingredient comprised in said drug delivery system, which is preferably a thermosensitive liposome according to the invention, a further thermosensitive liposome according to the invention, a composition according to the invention or a further composition according to the invention, is transferred from said drug delivery system to said target.

Regardless of the administration method, a thermosensitive liposome according to the invention, a further thermosensitive liposome according to the invention, a composition according to the invention or a further composition according to the invention will preferably circulate in the blood stream of said animal after administration. In order for said (further) thermosensitive liposome or said (further) composition to be delivered to said cell, said tissue, said organ, or said part thereof, it is required that said (further) thermosensitive liposome or said (further) composition is not cleared from the blood stream after a short period of time. In other words, it is required that said (further) thermosensitive liposome or said (further) composition has a long circulation half-time and/or does not lead to accelerated blood clearance (ABC). Both concepts relate to clearance, which is a pharmacokinetic measurement of the volume of plasma from which said (further) thermosensitive liposome or said (further) composition is completely removed per unit time.

A long circulation half-time means that the in vivo half-life of said active pharmaceutical ingredient in the bloodstream of an animal is higher than 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, 155 minutes, 160 minutes, 165 minutes, 170 minutes, 175 minutes, 180 minutes, 185 minutes, 190 minutes, 195 minutes, 200 minutes, 205 minutes, 210 minutes, 215 minutes, 220 minutes, 225 minutes, 230 minutes, 235 minutes, or 240 minutes. Herein, an animal is preferably a human, a rat, a cat, a dog or a pig. Preferably, the in vivo half-life of said active pharmaceutical ingredient in the bloodstream of an animal is from 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 125 minutes, 130 minutes, 135 minutes, 140 minutes, 145 minutes, 150 minutes, 155 minutes, 160 minutes, 165 minutes, 170 minutes, 175 minutes, 180 minutes, 185 minutes, 190 minutes, 195 minutes, 200 minutes, 205 minutes, 210 minutes, 215 minutes, 220 minutes, 225 minutes, 230 minutes, 235 minutes, or 240 minutes up to 720 minutes.

In a preferred embodiment is provided a thermosensitive liposome according to the invention, a further thermosensitive liposome according to the invention, a composition according to the invention or a further composition according to the invention having a long circulation half-time.

Accelerated blood clearance (ABC) is the phenomenon that a first administration of an active pharmaceutical ingredient or a drug delivery system to an animal is characterized by a significantly lower clearance than a subsequent administration within a given period of time. In other words, in the case of accelerated blood clearance said subsequent administration is characterized by a significantly higher clearance, i.e. an “accelerated” clearance. Without being bound to this theory, ABC may be due to an immunological response in said animal after said first administration. A subsequent administration is preferably a second, third, fourth, fifth, or sixth administration, relative to said first administration.

The absence of ABC is defined for a thermosensitive liposome according to the invention, a further thermosensitive liposome according to the invention, a composition according to the invention or a further composition according to the invention as the fact that the circulation half-time of said (further) thermosensitive liposome or said (further) composition after a first administration in an animal is not higher than 150%, or not higher than 140%, or not higher than 130%, or not higher than 120%, or not higher than 110%, or not higher than 105%, of the circulation half-time of said (further) thermosensitive liposome or said (further) composition after a second administration in said animal. Preferably, the absence of ABC is measured via the method outlined in Example 3, since it is well known to the skilled person that the circulation half-time may depend on the species or strain used.

Preferably, the absence of ABC is defined for a thermosensitive liposome according to the invention, a further thermosensitive liposome according to the invention, a composition according to the invention or a further composition according to the invention as the fact that the circulation half-time of said (further) thermosensitive liposome or said (further) composition after a first administration in an animal is not higher than 130% of the circulation half-time of said (further) thermosensitive liposome or said (further) composition after a second administration in said animal, wherein said second administration is less than 30 days, or less than 29 days, or less than 28 days, or less than 27 days, or less than 26 days, or less than 25 days, or less than 24 days, or less than 23 days, or less than 22 days, or less than 21 days, or less than 20 days, or less than 19 days, or less than 18 days, or less than 17 days, or less than 16 days, or less than 15 days, or less than 14 days, or less than 13 days, or less than 12 days, or less than 11 days, or less than 10 days, or less than 9 days, or less than 8 days, or less than 7 days, or less than 6 days, or less than 5 days, or less than 4 days, or less than 3 days, or less than 2 days, or less than 1 day after said first administration.

More preferably, the absence of ABC is defined for a thermosensitive liposome according to the invention, a further thermosensitive liposome according to the invention, a composition according to the invention or a further composition according to the invention as the fact that the circulation half-time of said (further) thermosensitive liposome or said (further) composition after two administrations in an animal is not higher than 150%, or not higher than 140%, or not higher than 130%, or not higher than 120%, or not higher than 110%, or not higher than 105%, of the circulation half-time of said (further) thermosensitive liposome or said (further) composition after a third administration in said animal.

Most preferably, the absence of ABC is defined for a thermosensitive liposome according to the invention, a further thermosensitive liposome according to the invention, a composition according to the invention or a further composition according to the invention as the fact that the circulation half-time of said (further) thermosensitive liposome or said (further) composition after a first administration in an animal is not higher than 130% of the circulation half-time of said (further) thermosensitive liposome or said (further) composition after a third administration in said animal, wherein said third administration is less than 30 days, or less than 29 days, or less than 28 days, or less than 27 days, or less than 26 days, or less than 25 days, or less than 24 days, or less than 23 days, or less than 22 days, or less than 21 days, or less than 20 days, or less than 19 days, or less than 18 days, or less than 17 days, or less than 16 days, or less than 15 days, or less than 14 days, or less than 13 days, or less than 12 days, or less than 11 days, or less than 10 days, or less than 9 days, or less than 8 days, or less than 7 days, or less than 6 days, or less than 5 days, or less than 4 days, or less than 3 days, or less than 2 days, or less than 1 day after said first administration.

Most preferably, the absence of ABC is defined for a thermosensitive liposome according to the invention, a further thermosensitive liposome according to the invention, a composition according to the invention or a further composition according to the invention as the fact that the circulation half-time of said (further) thermosensitive liposome or said (further) composition after a first administration in an animal is not higher than 130% of the circulation half-time of said (further) thermosensitive liposome or said (further) composition after a second, third, fourth, fifth or sixth administration in said animal, wherein said second, third, fourth, fifth or sixth administration is less than 30 days after said first administration.

In a preferred embodiment is provided a thermosensitive liposome according to the invention, a further thermosensitive liposome according to the invention, a composition according to the invention or a further composition according to the invention characterized by an absence of ABC.

Preferably, a thermosensitive liposome according to the invention, a further thermosensitive liposome according to the invention, a composition according to the invention or a further composition according to the invention is defined as having suitable clearance properties if administration of said (further) thermosensitive liposome or said (further) composition to an animal is characterized by a long circulation half-time and the absence of ABC. In a preferred embodiment is provided a thermosensitive liposome according to the invention, a further thermosensitive liposome according to the invention, a composition according to the invention or a further composition according to the invention having suitable clearance properties.

The delivery of an active pharmaceutical ingredient by a drug delivery system is preferably selective, meaning that the rate of the transfer of said active pharmaceutical ingredient from said drug delivery system to said target is significantly higher than the rate of the transfer of said active pharmaceutical ingredient from said drug delivery system to a corresponding cell, tissue, organ or part thereof, wherein said corresponding cell, tissue, organ or part thereof is of the same type as but not a part of said target, and/or higher than the rate of transfer to a cell, tissue, organ or part thereof surrounding said target. For example, selective delivery to a sarcoma target means that the rate of transfer of the active pharmaceutical ingredient to the heated sarcoma is higher than the rate of transfer to surrounding fat cells, muscle cells or tissues which are not part of the sarcoma and which have not heated. Herein, the heating defines the target. In the context of this application, this is defined as selective drug delivery. Herein, it is understood that the rate of transfer to non-corresponding cells, tissues, organs, or parts thereof, which are of another type and/or are not surrounding the target, may be higher than the rate of transfer to said target.

Selective drug delivery leads to a local accumulation of said active pharmaceutical ingredient in said target, wherein the concentration of said active pharmaceutical ingredient in said target is significantly higher than the concentration of said active pharmaceutical ingredient in a corresponding cell, tissue, organ or part thereof, wherein said corresponding cell, tissue, organ or part thereof is of the same type as but not a part of said target, preferably wherein said corresponding cell, tissue, organ or part thereof surrounds said target. In the context of this preferred definition, “significantly higher” preferably means at least 10% higher, at least 20% higher, at least 30% higher, at least 40% higher, at least 50% higher, at least 60% higher, at least 70% higher, at least 80% higher, at least 90% higher, at least 100% higher, at least 110% higher, at least 120% higher, at least 130% higher, at least 140% higher, at least 150% higher, at least 160% higher, at least 170% higher, at least 180% higher, at least 190% higher, or at least 200% higher, or at least 300% higher, or at least 400% higher, or at least 500% higher, or at least 600% higher, or at least 700% higher, or at least 800% higher, or at least 900% higher, or at least 1000% higher, or at least 1100% higher, or at least 1200% higher, or at least 1300% higher, or at least 1400% higher, or at least 1500% higher, or at least 1600% higher, or at least 1700% higher, or at least 1800% higher, or at least 1900% higher, or at least 2000% higher, or at least 2500% higher, or at least 3000% higher, or at least 3500% higher, or at least 4000% higher, or at least 4500% higher, or at least 5000% higher. Even more preferably, “significantly higher” in this context means from 10% higher, or from 20% higher, or from 30% higher, or from 40% higher, or from 50% higher, or from 60% higher, or from 70% higher, or from 80% higher, or from 90% higher, or from 100% higher, or from 110% higher, or from 120% higher, or from 130% higher, or from 140% higher, or from 150% higher, or from 160% higher, or from 170% higher, or from 180% higher, or from 190% higher, or from 200% higher, or from 300% higher, or from 400% higher, or from 500% higher, or from 600% higher, or from 700% higher, or from 800% higher, or from 900% higher, or from 1000% higher, or from 1100% higher, or from 1200% higher, or from 1300% higher, or from 1400% higher, or from 1500% higher, or from 1600% higher, or from 1700% higher, or from 1800% higher, or from 1900% higher, or from 2000% higher, or from 2500% higher, or from 3000% higher, or from 3500% higher, or at least 4000% higher, or from 4500% higher, or from 5000% higher, up to 10000% higher.

For a thermosensitive liposome according to the invention, a further thermosensitive liposome according to the invention, a composition according to the invention or a further composition according to the invention said selective drug delivery may be achieved by local heating of said target comprised in said animal after administration of said (further) thermosensitive liposome or said (further) composition to said animal. Above, it has been explained that release of the active pharmaceutical ingredient comprised in said (further) thermosensitive liposome or said (further) composition may occur at temperatures slightly above the body temperature of said animal. Hence, without being bound to this theory, the transfer of said active pharmaceutical ingredient from said (further) thermosensitive liposome or (further) composition may only occur at those cells, tissues, organs, or parts thereof which are locally heated to a temperature slightly above the body temperature of said animal.

It is understood that the body temperature of an animal, preferably of a human, refers to the normal body temperature of an animal of the same species which has not developed fever.

Selective delivery upon heat treatment is defined herein for a thermosensitive liposome according to the invention, a further thermosensitive liposome according to the invention, a composition according to the invention or a further composition according to the invention as the release of the active pharmaceutical ingredient comprised in said (further) thermosensitive liposome or (further) composition to said target, wherein said (further) thermosensitive liposome or (further) composition is first administered to said animal, wherein said target is then heated locally, wherein said local heating results in said release, leading to a concentration of said active pharmaceutical ingredient in said target which is higher than the concentration of said active pharmaceutical ingredient in a corresponding cell, tissue, organ, or part thereof, wherein said corresponding cell, tissue, organ or part thereof is of the same type as but not a part of said target, preferably wherein said corresponding cell, tissue, organ or part thereof surrounds said target.

In this sense, wherever the delivery or the release of an active pharmaceutical ingredient is described to be caused, triggered, induced, or facilitated by (local) heating or (local) heat treatment of said target, it should be understood that this means that the delivery or release of the active pharmaceutical ingredient is significantly higher in and near the target due to the elevated temperature caused by the heat treatment. This does not mean that no delivery or release, and does not mean that only negligible delivery or release, takes place in other cells, tissues, organs or parts thereof, comprised in the animal to which the drug delivery system is administered. Heating and heat treatment may be used interchangeably in the context of this application. In and near the target refers to those parts of said animal of which the temperature is increased, as defined above, after the heating of the target, relative to the temperature before said heating was applied.

A heat treatment preferably involves a light source such as a lamp or a near infrared laser (NIR), a water bath, a fluid recirculation device, a microwave device, radiofrequency ablation, and/or high-intensity focused ultrasound.

A preferred heat treatment involves heating said target for 5 minutes, or 6 minutes, or 7 minutes, or 8 minutes, or 9 minutes, or 10 minutes, or 11 minutes, or 12 minutes, or 13 minutes, or 14 minutes, or 15 minutes, or 16 minutes, or 17 minutes, or 18 minutes, or 19 minutes, or 20 minutes, or 21 minutes, or 22 minutes, or 23 minutes, or 24 minutes, or 25 minutes, or 26 minutes, or 27 minutes, or 28 minutes, or 29 minutes, or 30 minutes, or 31 minutes, or 32 minutes, or 33 minutes, or 34 minutes, or 35 minutes, or 36 minutes, or 37 minutes, or 38 minutes, or 39 minutes, or 40 minutes, or 41 minutes, or 42 minutes, or 43 minutes, or 44 minutes, or 45 minutes, or 46 minutes, or 47 minutes, or 48 minutes, or 49 minutes, or 50 minutes, or 51 minutes, or 52 minutes, or 53 minutes, or 54 minutes, or 55 minutes, or 56 minutes, or 57 minutes, or 58 minutes, or 59 minutes, or 60 minutes, or 65 minutes, or 70 minutes, or 75 minutes, or 80 minutes, or 85 minutes, or 90 minutes, or 95 minutes, or 100 minutes, or 105 minutes, or 110 minutes, or 115 minutes, or 120 minutes. Preferably, said preferred heat treatment involves heating said target to a temperature of 41° C.

A preferred heat treatment involves heating said target to a temperature of 40.0° C., or 40.1° C., or 40.2° C., or 40.3° C., or 40.4° C., or 40.5° C., or 40.6° C., or 40.7° C., or 40.8° C., or 40.9° C., or 41.0° C., or 41.1° C., or 41.2° C., or 41.3° C., or 41.4° C., or 41.5° C., or 41.6° C., or 41.7° C., or 41.8° C., or 41.9° C., or 42.0° C., or 42.1° C., or 42.2° C., or 42.3° C., or 42.4° C., or 42.5° C., or 42.6° C., or 42.7° C., or 42.8° C., or 42.9° C., or 43.0° C. In this context, around a given temperature means at a temperature higher than that given temperature minus 0.5° C. and lower than that given temperature plus 0.5° C., preferably at a temperature higher than that given temperature minus 0.2° C. and lower than that given temperature plus 0.2° C., more preferably at a temperature higher than that given temperature minus 0.1° C. and lower than that given temperature plus 0.1° C. Preferably, said preferred heat treatment involves heating said target for 60 minutes.

A more preferred heat treatment involves heating said target for 5 minutes, or 10 minutes, or 15 minutes, or 20 minutes, or 25 minutes, or 30 minutes at a temperature of 40° C. Another more preferred heat treatment involves heating said target for 5 minutes, or 10 minutes, or 15 minutes, or 20 minutes, or 25 minutes, or 30 minutes, or 35 minutes, or 40 minutes, or 45 minutes, or 50 minutes, or 55 minutes, or 60 minutes at a temperature of 40.0° C.

A more preferred heat treatment involves heating said target for 5 minutes, or 10 minutes, or 15 minutes, or 20 minutes, or 25 minutes, or 30 minutes at a temperature of 40° C. Another more preferred heat treatment involves heating said target for 5 minutes, or 10 minutes, or 15 minutes, or 20 minutes, or 25 minutes, or 30 minutes, or 35 minutes, or 40 minutes, or 45 minutes, or 50 minutes, or 55 minutes, or 60 minutes at a temperature of 41.0° C.

A more preferred heat treatment involves heating said target for 5 minutes, or 10 minutes, or 15 minutes, or 20 minutes, or 25 minutes, or 30 minutes at a temperature of 40° C. Another more preferred heat treatment involves heating said target for 5 minutes, or 10 minutes, or 15 minutes, or 20 minutes, or 25 minutes, or 30 minutes, or 35 minutes, or 40 minutes, or 45 minutes, or 50 minutes, or 55 minutes, or 60 minutes at a temperature of 42.0° C.

A more preferred heat treatment involves heating said target for 5 minutes, or 10 minutes, or 15 minutes, or 20 minutes, or 25 minutes, or 30 minutes at a temperature of 40° C. Another more preferred heat treatment involves heating said target for 5 minutes, or 10 minutes, or 15 minutes, or 20 minutes, or 25 minutes, or 30 minutes, or 35 minutes, or 40 minutes, or 45 minutes, or 50 minutes, or 55 minutes, or 60 minutes at a temperature of 43.0° C.

A thermosensitive liposome according to the invention, a further thermosensitive liposome according to the invention, a composition according to the invention or a further composition according to the invention has or is characterized by a suitable biodistribution if, after administration of said (further) thermosensitive liposome or (further) composition to an animal, the active pharmaceutical ingredient comprised therein is delivered selectively upon heat treatment, as defined above, to said target. Preferably, said target is a tumor cell or a tumor, and said cell, tissue or organ not comprised in said target is heart, liver, spleen, kidney, lung or muscle without heat treatment. A suitable biodistribution can be determined as outlined in Example 4.

In a preferred embodiment is provided a thermosensitive liposome according to the invention, a further thermosensitive liposome according to the invention, a composition according to the invention or a further composition according to the invention characterized by a suitable biodistribution.

The selective delivery of an active pharmaceutical ingredient from a drug delivery system upon heat treatment is preferably high, meaning that a larger fraction of the active pharmaceutical ingredient comprised in said drug delivery system is transferred to said target than to a corresponding cell, tissue, organ or part thereof, wherein said corresponding cell, tissue, organ or part thereof is of the same type as but not a part of said target, preferably wherein said cell, tissue, organ or part thereof surrounds said target, after administration of said drug delivery system to an animal. In the context of this preferred definition, “larger” preferably means at least 10% more, at least 20% more, at least 30% more, at least 40% more, at least 50% more, at least 60% more, at least 70% more, at least 80% more, at least 90% more, at least 100% more, at least 110% more, at least 120% more, at least 130% more, at least 140% more, at least 150% more, at least 160% more, at least 170% more, at least 180% more, at least 190% more, or at least 200% more, or at least 300% more, or at least 400% more, or at least 500% more, or at least 600% more, or at least 700% more, or at least 800% more, or at least 900% more, or at least 1000% more, or at least 1100% more, or at least 1200% more, or at least 1300% more, or at least 1400% more, or at least 1500% more, or at least 1600% more, or at least 1700% more, or at least 1800% more, or at least 1900% more, or at least 2000% more, or at least 2500% more, or at least 3000% more, or at least 3500% more, or at least 4000% more, or at least 4500% more, or at least 5000% more. Even more preferably, larger means from 200% more up to 10000% more, from 300% more up to 10000% more, from 400% more up to 10000% more, from 500% more up to 10000% more, from 600% more up to 10000% more, from 700% more up to 10000% more, from 800% more up to 10000% more, from 900% more up to 10000% more, from 1000% more up to 10000% more, from 1500% more up to 10000% more, from 2000% more up to 10000% more, from 2500% more up to 10000% more, from 3000% more up to 10000% more, from 3500% more up to 10000% more, from 4000% more up to 10000% more, from 4500% more up to 10000% more, or from 5000% more up to 10000% more.

Preferably, a drug delivery system is a thermosensitive liposome according to the invention, a further thermosensitive liposome according to the invention, a composition according to the invention or a further composition according to the invention. In this context, the terms (drug) delivery (of an active pharmaceutical ingredient), release (of an active pharmaceutical ingredient), transfer (of an active pharmaceutical ingredient) from said drug delivery system, (further) thermosensitive liposome or (further) compositions can be used interchangeably.

A drug delivery system, which is a pharmaceutical composition, is preferably stable upon storage. In the case a drug delivery system is a thermosensitive liposome according to the invention, a further thermosensitive liposome according to the invention, a composition according to the invention or a further composition according to the invention stability means that:
the concentration of the active pharmaceutical ingredient comprised in said (further) thermosensitive liposome or said (further) composition does not change by more than 15% upon storage, preferably by not more 10%, most preferably by not more than 5%, relative to the concentration of the active pharmaceutical ingredient before storage; and/or
the diameter of said (further) thermosensitive liposome or the diameter of said (further) thermosensitive liposome comprised in said composition does not change by more than 30% upon storage, preferably by not more 20%, most preferably by not more than 10%, relative to the diameter of said (further) thermosensitive liposomes before storage; and/or
the concentration in the bilayer of lysolipids comprised in said (further) thermosensitive liposome or said (further) composition does not increase above 5%, more preferably not above 2%, most preferably not above 1%, during storage; and/or
the polydispersity of said (further) thermosensitive liposome or the polydispersity of said (further) thermosensitive liposome comprised in said (further) composition does not increase above 0.5, preferably not above 0.3, more preferably not above 0.2, during storage, and/or
if said (further) thermosensitive liposome or said (further) composition is characterized before storage by a high selective delivery upon heat treatment, then said (further) thermosensitive liposome or said (further) composition is still characterized by a high selective delivery upon heat treatment after storage; and/or
if said (further) thermosensitive liposome or said (further) composition is a suitable drug delivery system before storage, then said (further) thermosensitive liposome or said (further) composition is still a suitable drug delivery system after storage.

The concentration of the active pharmaceutical ingredient and the lysolipids may be determined via H PLC. The diameter and polydispersity may be determined via dynamic light scattering. High selective delivery and suitable drug delivery systems are described above.

In the context of this application, storage of a drug delivery system, a thermosensitive liposome according to the invention, a further thermosensitive liposome according to the invention, a composition according to the invention or a further composition according to the invention is a period between the preparation of said drug delivery system, (further) thermosensitive liposome or (further) composition and the administration thereof to an animal. It is hereby understood that stable “upon storage” may imply that said drug delivery system, (further) thermosensitive liposome or (further) composition is stored under a specific set of storage conditions such as a lowered temperature.

Preferably, storage is for at least 1 day, or for at least 2 days, or for at least 3 days, or for at least 4 days, or for at least 5 days, or for at least 6 days, or for at least 7 days, or for at least 8 days, or for at least 9 days, or for at least 10 days, or for at least 11 days, or for at least 12 days, or for at least 13 days, or for at least 14 days, or for at least 15 days, or for at least 16 days, or for at least 17 days, or for at least 18 days, or for at least 19 days, or for at least 20 days, or for at least 21 days, or for at least 22 days, or for at least 23 days, or for at least 24 days, or for at least 25 days, or for at least 26 days, or for at least 27 days, or for at least 28 days, or for at least 29 days, or for at least 30 days, or for at least 5 weeks, or for at least 6 weeks, or for at least 7 weeks, or for at least 8 weeks, or for at least 9 weeks, or for at least 10 weeks, or for at least 11 weeks, or for at least 12 weeks, or for at least 13 weeks, or for at least 14 weeks, or for at least 15 weeks, or for at least 16 weeks, or for at least 5 months, or for at least 6 months, or for at least 7 months, or for at least 8 months, or for at least 9 months, or for at least 10 months, or for at least 11 months, or for at least 12 months, or for at least 13 months, or for at least 14 months, or for at least 15 months, or for at least 16 months, or for at least 17 months, or for at least 18 months, or for at least 19 months, or for at least 20 months, or for at least 21 months, or for at least 22 months, or for at least 23 months, or for at least 24 months, or for at least 25 months, or for at least 26 months, or for at least 27 months, or for at least 28 months, or for at least 29 months, or for at least 30 months.

Preferably, storage is from or from 1 day, or from 2 days, or from 3 days, or from 4 days, or from 5 days, or from 6 days, or from 7 days, or from 8 days, or from 9 days, or from 10 days, or from 11 days, or from 12 days, or from 13 days, or from 14 days, or from 15 days, or from 16 days, or from 17 days, or from 18 days, or from 19 days, or from 20 days, or from 21 days, or from 22 days, or from 23 days, or from 24 days, or from 25 days, or from 26 days, or from 27 days, or from 28 days, or from 29 days, or from 30 days, or from 5 weeks, or from 6 weeks, or from 7 weeks, or from 8 weeks, or from 9 weeks, or from 10 weeks, or from 11 weeks, or from 12 weeks, or from 13 weeks, or from 14 weeks, or from 15 weeks, or from 16 weeks, or from 5 months, or from 6 months, or from 7 months, or from 8 months, or from 9 months, or from 10 months, or from 11 months, or from 12 months, or from 13 months, or from 14 months, or from 15 months, or from 16 months, or from 17 months, or from 18 months, or from 19 months, or from 20 months, or from 21 months, or from 22 months, or from 23 months, or from 24 months, or from 25 months, or from 26 months, or from 27 months, or from 28 months, or from 29 months, or from 30 months, up to 5 years.

Preferably, storage occurs at a temperature around 25° C., or around 20° C., or around 15° C., or around 10° C., or around 5° C., or around 0° C., or around −5° C., or around −10° C., or around −15° C., or around −20° C. In this context, around a given temperature means at a temperature higher than that given temperature minus 5° C. and lower than that given temperature plus 5° C., preferably at a temperature higher than that given temperature minus 2° C. and lower than that given temperature plus 2° C., more preferably at a temperature higher than that given temperature minus 1° C. and lower than that given temperature plus 1° C. Preferably, at a temperature around −20° C. means at a temperature from −15° C. up to −25° C.

If storage takes place at a temperature lower than 0° C., stability also implies that the stability conditions are met during and after freezing and thawing of the drug delivery system, the (further) thermosensitive liposome according to the invention or the (further) composition according to the invention. This property is also called freezing and thawing stability, and may encompass several freezing-thawing cycles.

Preferably, a thermosensitive liposome according to the invention, a further thermosensitive liposome according to the invention, a composition according to the invention or a further composition according to the invention is stable upon at least 1, 2, 3, 4, 5, 6, 7, 8, 9, 10, 11, 12, 13, 14, 15, 16, 17, 18, 19, 20 freezing-thawing cycles.

Dispersion stability of a thermosensitive liposome according to the invention, a further thermosensitive liposome according to the invention, a composition according to the invention or a further composition according to the invention means that the diameter of said (further) thermosensitive liposome or the diameter of said (further) thermosensitive liposome comprised in said (further) composition does not change by more than 30% upon storage, preferably by not more 20%, most preferably by not more than 10%, relative to the diameter of said (further) thermosensitive liposomes before storage.

Leakage of an active pharmaceutical ingredient from a thermosensitive liposome according to the invention, a further thermosensitive liposome according to the invention, a composition according to the invention or a further composition according to the invention is the transfer from said (further) thermosensitive liposome, or from a (further) thermosensitive liposome comprised in said (further) composition, upon storage, leading to a decrease of the concentration of the active pharmaceutical ingredient therein. Preferably, the concentration decrease of the active pharmaceutical ingredient due to leakage is not more than 15% upon storage, more preferably not more than 10%, most preferably not more than 5%, relative to the total concentration of the active pharmaceutical ingredient before storage.

Degradation of active pharmaceutical ingredient in a thermosensitive liposome according to the invention, a further thermosensitive liposome according to the invention, a composition according to the invention or a further composition according to the invention is the decrease of the concentration of said active pharmaceutical ingredient in said (further) thermosensitive liposome or said (further) composition, without the transfer of said active pharmaceutical ingredient across the bilayer of said (further) thermosensitive liposome or across the bilayer of a thermosensitive liposome comprised in said (further) composition. In other words, degradation involves a chemical transformation whereby an active pharmaceutical ingredient is converted into a molecule with no, a lowered, a different, or an adverse pharmaceutical effect. Preferably, the concentration decrease of the active pharmaceutical ingredient due to degradation is not more than 15% upon storage, more preferably not more than 10%, most preferably not more than 5%, relative to the concentration of the active pharmaceutical ingredient before storage.

The term “upon storage” preferably means at least 4 weeks at a temperature around 5° C. or for at least 12 months at a temperature around −20° C. More preferably, upon storage preferably means from 4 weeks up to 20 weeks at a temperature around 5° C. or from 12 months up to 16 months at a temperature around −20° C.

In a preferred embodiment is provided a thermosensitive liposome, a further thermosensitive liposome, a composition or a further composition according to the invention which is stable upon storage for at least 4 weeks at a temperature around 5° C., or for at least 12 months at a temperature around −20° C., or for from 4 weeks up to 20 weeks at a temperature around 5° C., or for from 12 months up to 16 months at a temperature around −20° C., wherein
the concentration of the active pharmaceutical ingredient comprised in said (further) thermosensitive liposome or said (further) composition does not change by more than 15% upon storage, preferably by not more 10%, most preferably by not more than 5%, relative to the concentration of the active pharmaceutical ingredient before storage; and
the diameter of said (further) thermosensitive liposome or the diameter of said (further) thermosensitive liposome comprised in said composition does not change by more than 30% upon storage, preferably by not more 20%, most preferably by not more than 10%, relative to the diameter of said (further) thermosensitive liposomes before storage; and
the concentration in the bilayer of lysolipids comprised in said (further) thermosensitive liposome or said (further) composition does not increase above 5%, more preferably not above 2%, most preferably not above 1%, during storage; and
the polydispersity of said (further) thermosensitive liposome or the polydispersity of said (further) thermosensitive liposome comprised in said composition does not increase above 0.5, preferably not above 0.3, more preferably not above 0.2 during storage; and
if said (further) thermosensitive liposome or said (further) composition is characterized before storage by a high selective delivery upon heat treatment, then said (further) thermosensitive liposome or said (further) composition is still characterized by a high selective delivery upon heat treatment after storage; and
if said (further) thermosensitive liposome or said (further) composition is a suitable drug delivery system before storage, then said (further) thermosensitive liposome or said (further) composition is still a suitable drug delivery system after storage.

A thermosensitive liposome, a further thermosensitive liposome, a composition or a further composition according to this embodiment is called highly stable upon storage or a (further) thermosensitive liposome or a (further) composition having a high stability upon storage in the context of this application.

In a preferred embodiment is provided a thermosensitive liposome, a further thermosensitive liposome, a composition or a further composition according to the invention which is stable upon storage from 1 up to 20 weeks at a temperature around 5° C., or from 1 up to 16 months at a temperature around −20° C., or from 12 up to 16 months at a temperature around −20° C., wherein the concentration of the active pharmaceutical ingredient comprised in said (further) thermosensitive liposome or said (further) composition does not change by more than 15% upon storage, and/or wherein the diameter of said (further) thermosensitive liposome or the diameter of said (further) thermosensitive liposome comprised in said composition does not change by more than 30% upon storage, wherein the concentration in the bilayer of lysolipids comprised in said (further) thermosensitive liposome or said (further) composition does not increase above 5%, and/or wherein the polydispersity of said thermosensitive liposome or the polydispersity of said (further) thermosensitive liposome comprised in said (further) composition does not increase above 0.5. Preferably, if said (further) thermosensitive liposome or said (further) composition is characterized before storage by a high selective delivery upon heat treatment, then said (further) thermosensitive liposome or said (further) composition is still characterized by a high selective delivery upon heat treatment after storage, and/or wherein if said (further) thermosensitive liposome or said (further) composition is a suitable drug delivery system before storage, then said (further) thermosensitive liposome or said (further) composition is still a suitable drug delivery system after storage.

Unlike the thermosensitive liposomes mentioned in prior art document [4] and [5], the examples below clearly show that a thermosensitive liposome according to the invention is stable upon storage. Specifically, Example 7 shows that at least the intraliposomal API:lipid ratio of 0.05 to 0.3, characteristic of a thermosensitive liposome according to the invention, is associated with a stability upon storage for at least 4 weeks at 2-8° C. In contrast, [4] states that “Storing TSL with encapsulated 300 mM citrate, pH 4 at 4° C. for weeks yielded in hydrolysis of the phospholipids and the generation of lysolipids”, whereas [5] mentions that “No Lyso-PC formation was detectable during the first 20 min of loading, but content increased with time to 1.1%±1.2% after 60 min.”

Moreover, several preferred aspects and embodiments of thermosensitive liposomes according to the invention, as described above, are associated with an even higher stability upon storage:
Example 5 shows that the use of an intraliposomal pH in the range between 6.0 to 7.4 ensured phospholipid stability for at least 4 weeks at 2-8° C. A corresponding formulation with an intraliposomal pH of 3 showed more than 10% of lipid degradation products after 12 weeks storage at 2-8° C. (28.16±1.95% free fatty acids and 14.1±5.1% lysolipids), whereas for the formulation with pH 6 no change was observed in the analyzed 24 weeks. The API leakage during storage also increased after 12 weeks for the formulation with pH 3, whereas no leakage was detectable for the pH 6 formulation. In line with this finding, Example 7 also shows that, a more neutral pH used as intraliposomal buffer leads to a slower lipid excipient decomposition at 2-8° C. than with a more acidic pH.
Example 8 shows stability upon storage at 2-8° C. if the diameter of the thermosensitive liposome is from 100 nanometers up to 200 nanometers. For example, reducing vesicle size to a z average below 100 nm, the formulation is prone to API and lipid excipient decomposition which in turn negatively affects the temperature-dependent release profile.
Example 9 shows stability upon storage at 2-8° C. if a storage buffer is used with a saline concentration up to 100 mM and an osmolarity of at least 300 mOsmol/kg. For example, reducing saline concentration in the storage buffer from physiological concentration (140 mM) to 66 mM stabilized the DPPG₂-TSL_{30%}-DOX for storage at 2-8° C., resulting in a dispersion that showed a notably slower increase in vesicle size.
Example 9 further shows that the presence of a cryoprotectant such as trehalose increase the stability upon storage. Specifically, at least the presence of 8% (w/v) of trehalose is associated with a composition that is stable upon at least 6 freezing-thawing cycles.

Additionally, Example 6 demonstrates that using a salt concentration of up to 120 mM in the loading buffer accelerates active loading of doxorubicin and therefore reduces decomposition of API and lipids. Total API-related impurity content in the batch dependent on incubation time, with <0.10 Area % and 4.1 Area % for 30 min and 90 min, respectively. Total lipid-related impurity content in the batch also dependent on incubation time, with <0.10 Area % and 0.13 Area % for 30 min and 90 min, respectively. To achieve storage of the formulation, a buffer exchange to a storage buffer with a salt concentration lower than 100 mM and an osmolarity higher than 300 mOsmol/kg is preferred.

### Anti-Cancer Activity

A drug delivery system described in this application is preferably for delivering an anticancer drug to a tumor, preferably a solid tumor. Preferably, said drug delivery system comprises a thermosensitive liposome and said delivery is induced or facilitated by heating of said tumor.

In a preferred embodiment is provided a thermosensitive liposome according to the invention, a further thermosensitive liposome according to the invention, a composition according to the invention or a further composition according to the invention, wherein said (further) thermosensitive liposome or (further) composition exhibits a detectable anti-tumor activity. Within the context of this invention, an anti-tumor activity is only seen in a tumor or in a tumor cell comprised therein, and not in a corresponding healthy, control, reference, non-tumor cell, tissue or organ. Within the context of the invention, an anti-tumor activity comprises at least one of the following:
a decrease of cell viability said tumor cell,
an induction of apoptosis in said tumor cell or an induction of tumor cell death,
an inhibition of proliferation of said tumor cell,
an inhibition or delay of the weight increase, or a decrease of the weight, and/or a delayed or inhibited growth of said tumor.

Exhibiting such a detectable anti-tumor activity is crucial in the present invention in order to be able to treat, ameliorate, delay, cure and/or prevent cancer, preferably solid tumors. The terms anti-tumor activity or an effect in a tumor cell are used interchangeably in the context of this invention. It is clear that the use of the term an anti-tumor activity or effect, and an effect in a tumor cell in regard to a thermosensitive liposome according to the invention, a further thermosensitive liposome according to the invention, a composition according to the invention or a further composition according to the invention do not imply that said (further) thermosensitive liposome or (further) composition enter said tumor or tumor cell. As described above, it is well understood that said (further) thermosensitive liposome or (further) composition may function as a drug delivery system for an active pharmaceutical ingredient with such an anti-tumor activity.

The assessment of an anti-tumor activity may be carried out periodically, e.g. each week, two weeks, three weeks, four weeks, one month, two months, three months, four months, five months, six months or each year in a treated subject.

The increase/decrease of an anti-tumor activity may therefore be assessed periodically, e.g. each week, month. This assessment is preferably carried out at several time points for a given subject or at one or several time points for a given subject and a healthy control. Alternatively, such anti-tumor activity may be measured by comparing said anti-tumor activity in a tumor cell from a subject with the corresponding activity in a non-tumor or healthy cell from the same subject at a given time point after start of treatment.

When an anti-tumor activity has been detected at least once, twice, or three times, a thermosensitive liposome according to the invention, a further thermosensitive liposome according to the invention, a composition according to the invention or a further composition according to the invention is said to exhibit a detectable an anti-tumor activity.

A detectable anti-tumor activity has therefore been preferably detected when for at least one time point, an anti-tumor activity has been detected. Preferably, such a detectable anti-tumor activity has been detected for at least two, three, four, five time points. In a preferred embodiment, an anti-tumor activity is assessed in tumor cells of a subject. More preferably, said tumor cells are sarcoma cells or carcinoma cells. In a preferred embodiment, said carcinoma cells are lung carcinomas, hepatocellular carcinomas, or colon carcinomas. In a more preferred embodiment, said carcinoma cells are lung carcinomas, or hepatocellular carcinomas. In a preferred embodiment, said sarcoma cells are angiosarcoma, bone sarcoma, dermatofibrosarcoma protuberans, epithelioid sarcoma, gastrointestinal stromal tumor (GIST), kaposi's sarcoma, leiomyosarcoma, liposarcoma, malignant peripheral nerve sheath tumors, myxofibrosarcoma, pleomorphic sarcoma, rhabdomyosarcoma, solitary fibrous tumor, synovial sarcoma or undifferentiated pleomorphic sarcoma.

A decrease of tumor cell viability or survival may be at least a decrease of at least 1%, 5%, 10%, 15%, 20%, 25%, 30%, 40%, 50%, 55%, 60%, 65%, 70% or 75%, or more, with reference to the corresponding tumor cell viability or survival of a tumor cell wherein a control thermosensitive liposome, as defined hereafter, was introduced.

An induction of apoptosis in tumor cells or an induction of tumor cell death may be at least 1%, 5%, 10%, 15%, 20%, 25%, 30%, 40%, 50%, 55%, 60%, 65%, 70% or 75%, or more. For example, an induction of apoptosis in tumor cells or an induction of tumor cell death of 50% means that half of said tumor cells treated with a thermosensitive liposome according to the invention, a further thermosensitive liposome according to the invention, a composition according to the invention or a further composition according to the invention undergo apoptosis or cell death. Tumor cell viability or survival or death may be assessed using techniques known to the skilled person. Tumor cell viability and death may be assessed using routine imaging methods such as MRI, CT or PET, and derivatives thereof, or in biopsies. Tumor cell viability may be assessed by visualizing the extension of the lesion at several time points. A decrease of 10%, 15%, 20%, 25%, 30%, 40%, 50%, 55%, 60%, 65%, 70% or 75%, or more, with reference to the extension of the lesion at the first time point, of the lesion observed at least once will be seen as a decrease of tumor cell viability. Tumor cell viability may be assessed through an indirect ATP measurement such as the CellTiter-Glo kit from Promega. Tumor cell apoptosis may be assessed by measuring caspase-3/7 activity.

An inhibition of the proliferation of tumor cells may be at least 1%, 5%, 10%, 15%, 20%, 30%, 40%, 50%, 55%, 60%, 65%, 70% or 75%, or more, with reference to the corresponding proliferation of a tumor cell wherein a control thermosensitive liposome, as defined hereafter, was introduced. Proliferation of cells may be assessed using known techniques as a standard proliferation assay. Such a proliferation assay may use of vital stains such as Cell Titer Blue (Promega). This includes a substrate molecule that is converted into a fluorescent molecule by metabolic enzymes. The level of fluorescence then reflects the number of living and metabolically active cells. Alternatively, such proliferation assay may determine the mitotic index. The mitotic index is based on the number of tumor cells under proliferation stage compared to the number of total tumor cells. The labelling of proliferative cells can be performed by using the antibody Ki-67 and immunohistochemistry staining. An inhibition of the proliferation of tumors cells may be seen when the mitotic index is reduced by at least 20%, at least 30%, at least 50% or more (as described in Kearsley J. H., et al, 1990).

In certain embodiments, an inhibition or a decrease of a tumor weight or a delayed tumor growth or an inhibition of a tumor growth may be of at least 1%, 5%, 10%, 15%, 20%, 30%, 40%, 50%, 55%, 60%, 65%, 70% or 75%, or more, with reference to a tumor wherein a control thermosensitive liposome, as defined hereafter, was introduced. Tumor weight or tumor growth may be assessed using techniques known to the skilled person.

The detection of tumor growth or the detection of the proliferation of tumor cells may be assessed in vivo by measuring changes in glucose utilization by positron emission tomography with the glucose analogue 2-[18F]-fluor-2-deoxy-D-glucose (FDG-PET) or [18F]-′3-fluoro-′3-deoxy-L-thymidine PET. An ex vivo alternative may be staining of a tumor biopsy with Ki67.

In a preferred embodiment is provided a thermosensitive liposome according to the invention, a further thermosensitive liposome according to the invention, a composition according to the invention or a further composition according to the invention for use as a medicament.

In a more preferred embodiment is provided a thermosensitive liposome according to the invention, a further thermosensitive liposome according to the invention, a composition according to the invention or a further composition according to the invention for use in treating, ameliorating, delaying, curing and/or preventing cancer.

A preferred cancer is selected from the group consisting of leukemia, Hodgkin's lymphoma, bladder cancer, breast cancer, stomach cancer, lung cancer, ovarian cancer, thyroid cancer, pancreatic cancer, head and neck cancer, prostate cancer, gastrointestinal cancer, soft tissue sarcoma, and multiple myeloma. More preferably, a cancer is a soft tissue sarcoma.

Preferably, a soft tissue sarcoma is selected from the group consisting of angiosarcoma, bone sarcoma, dermatofibrosarcoma protuberans, epithelioid sarcoma, gastrointestinal stromal tumor (GIST), kaposi's sarcoma, leiomyosarcoma, liposarcoma, malignant peripheral nerve sheath tumors, myxofibrosarcoma, pleomorphic sarcoma, rhabdomyosarcoma, solitary fibrous tumor, synovial sarcoma and undifferentiated pleomorphic sarcoma.

In a preferred embodiment is provided a thermosensitive liposome according to the invention, a further thermosensitive liposome according to the invention, a composition according to the invention or a further composition according to the invention for use in treating, ameliorating, delaying, curing and/or preventing soft tissue sarcoma.

In a preferred embodiment is provided a thermosensitive liposome according to the invention, a further thermosensitive liposome according to the invention, a composition according to the invention or a further composition according to the invention for use in treating, ameliorating, delaying, curing and/or preventing cancer, preferably soft tissue sarcoma, wherein said (further) thermosensitive liposome or said (further) composition has an anti-tumor activity, and wherein said (further) thermosensitive liposome or said (further) composition has one or more of the following properties:
a low complement activation,
the absence of an anaphylaxis,
non-toxicity,
a long circulation half-time,
the absence of ABC,
a suitable clearance,
a suitable biodistribution,
a high selective delivery upon heat treatment
a high stability upon storage.

In a preferred embodiment is provided a thermosensitive liposome according to the invention, a further thermosensitive liposome according to the invention, a composition according to the invention or a further composition according to the invention for use in treating, ameliorating, delaying, curing and/or preventing cancer, preferably soft tissue sarcoma, wherein said (further) thermosensitive liposome or said (further) composition has an anti-tumor activity, and wherein said (further) thermosensitive liposome or said (further) composition is a suitable drug delivery system. In other words, a (further) thermosensitive liposome or a (further) composition according to this embodiment has an anti-tumor activity, has a suitable clearance and a high stability upon storage.

Criteria to judge therapeutic response are known as the RECIST (Wahl R. L. et al, 2009) criteria. In the context of the invention, a patient may survive and/or may be considered as remaining disease free for a longer time interval. Alternatively, the disease or condition may have been stopped or delayed. In the context of the invention, an improvement of quality of life and observed pain relief may mean that a patient may need less pain relief drugs than at the onset of the treatment. Alternatively or in combination with the consumption of less pain relief drugs, a patient may be less constipated than at the onset of the treatment. “Less” in this context may mean 5% less, 10% less, 20% less, 30% less, 40% less, 50% less, 60% less, 70% less, 80% less, 90% less. A patient may no longer need any pain relief drug. This improvement of quality of life and observed pain relief may be seen, detected or assessed after at least one week, two weeks, three weeks, four weeks, one month, two months, three months, four months, five months, six months or more of treatment in a patient and compared to the quality of life and observed pain relief at the onset of the treatment of said patient.

A delay in occurrence of metastases and/or of tumor cell migration may be a delay of at least one week, one month, several months, one year or longer. The presence of metastases may be assessed using MRI, CT or echography or techniques allowing the detection of circulating tumor cells (CTC), cell-free DNA or cell-free RNA. Examples of the latter tests are CellSearch CTC test (Veridex), an EpCam-based magnetic sorting of CTCs from peripheral blood.

In certain embodiments, tumor growth may be delayed at least one week, one month, two months or more. In a certain embodiment, an occurrence of metastases is delayed at least one week, two weeks, three weeks, four weeks, one months, two months, three months, four months, five months, six months or more.

### Use

In a further aspect, there is provided the use of a thermosensitive liposome according to the invention, a further thermosensitive liposome according to the invention, a composition according to the invention or a further composition according to the invention as described in the previous sections for use as a medicament or part of therapy. There is also provided a use of a thermosensitive liposome according to the invention, a further thermosensitive liposome according to the invention, a composition according to the invention or a further composition according to the invention as described in the previous section for the manufacture of a medicament for treating cancer, preferably soft tissue sarcoma.

Preferably, a thermosensitive liposome according to the invention, a further thermosensitive liposome according to the invention, a composition according to the invention or a further composition according to the invention is for use as a medicament or part of a therapy for preventing, delaying, curing, ameliorating and/or treating cancer, preferably soft tissue sarcoma.

In an embodiment of this aspect of the invention is provided a thermosensitive liposome according to the invention, a further thermosensitive liposome according to the invention, a composition according to the invention or a further composition according to the invention for use as a medicament, preferably for treating, preventing, and/or delaying cancer, preferably soft tissue sarcoma.

### Method

In a further aspect, there is provided a method for preventing, treating, curing, ameliorating and/or delaying a condition or disease as defined in the previous section in an individual, in a cell, tissue or organ of said individual. The method comprises administering a thermosensitive liposome according to the invention, a further thermosensitive liposome according to the invention, a composition according to the invention or a further composition according to the invention to said individual or a subject in the need thereof.

Any preference or preferred embodiment disclosing a (further) thermosensitive liposome according to the invention, or disclosing a (further) composition comprising a (further) thermosensitive liposome according to the invention, or disclosing a method according to the invention, may be combined with a method for preventing, treating, curing, ameliorating and/or delaying a condition or disease in an individual, in a cell, tissue or organ of said individual, meaning that any such preference or preferred embodiments also discloses a corresponding method for preventing, treating, curing, ameliorating and/or delaying a condition or disease.

In an embodiment of this aspect of the invention is provided a method for preventing, treating, and/or delaying cancer, preferably soft tissue sarcoma, comprising administering to a subject a thermosensitive liposome according to the invention, a further thermosensitive liposome according to the invention, a composition according to the invention or a further composition according to the invention.

In a preferred embodiment, a thermosensitive liposome according to the invention, a further thermosensitive liposome according to the invention, a composition according to the invention or a further composition according to the invention is administered to a cell present in an organ or in a tissue wherein a tumor is present. Preferably, said organ or tissue comprises 1%, 5%, 10%, 15%, 20%, 30%, 40%, 50%, 60%, 70%, 80%, 90%, 100% tumor cells. A thermosensitive liposome according to the invention, a further thermosensitive liposome according to the invention, a composition according to the invention or a further composition according to the invention may be targeted to tumor cells, e.g. by coupling or conjugating a bilayer comprised in said thermosensitive liposome with an antibody or other moiety binding to the tumor. Preferably, said tumor is associated with soft tissue sarcoma.

In a preferred embodiment, a thermosensitive liposome according to the invention, a further thermosensitive liposome according to the invention, a composition according to the invention or a further composition according to the invention is administered to a cell present in an organ or in a tissue wherein a tumor is present, wherein said tumor has not yet metastasized. In another preferred embodiment, a thermosensitive liposome according to the invention, a further thermosensitive liposome according to the invention, a composition according to the invention or a further composition according to the invention is administered to a cell present in an organ or in a tissue wherein a tumor is present, wherein said tumor has metastasized. Preferably, said tumor is associated with soft tissue sarcoma.

In a preferred embodiment, a thermosensitive liposome according to the invention, a further thermosensitive liposome according to the invention, a composition according to the invention or a further composition according to the invention is administered systemically. Alternatively, in another embodiment, the treatment is locally administered.

In another preferred embodiment, a thermosensitive liposome according to the invention, a further thermosensitive liposome according to the invention, a composition according to the invention or a further composition according to the invention may be administered in combination with standard treatments of disease or condition associated with cancer, preferably with soft tissue sarcoma, such as chemotherapy, radiotherapy or surgery.

### Definitions

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a (thermosensitive) liposome or a composition as defined herein may comprise additional component(s) than the ones specifically identified, said additional component(s) not altering the unique characteristic of the invention. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Each embodiment as identified herein may be combined together unless otherwise indicated. All patent and literature references cited in the present specification are hereby incorporated by reference in their entirety.

In the context of this invention “identical” should not be so narrowly construed as to imply that the natural abundance of isotopes should be contemplated—identical should preferably only refer to the molecular structure as would be represented in a drawn structural formula.

Whenever a parameter of a substance is discussed in the context of this invention, it is assumed that unless otherwise specified, the parameter is determined, measured, or manifested under physiological conditions. Physiological conditions are known to a person skilled in the art, and comprise aqueous solvent systems, atmospheric pressure, pH-values from 6 to 8, a temperature ranging from room temperature to about 37° C. (from about 20° C. to about 40° C.), and a suitable concentration of buffer salts or other components. It is understood that charge is often associated with equilibrium. A moiety that is said to carry or bear a charge is a moiety that will be found in a state where it bears or carries such a charge more often than that it does not bear or carry such a charge. As such, an atom that is indicated in this disclosure to be charged could be non-charged under specific conditions, and a neutral moiety could be charged under specific conditions, as is understood by a person skilled in the art.

In the context of this invention, a decrease or increase of a parameter to be assessed means a change of at least 5% of the value corresponding to that parameter. More preferably, a decrease or increase of the value means a change of at least 10%, even more preferably at least 20%, at least 30%, at least 40%, at least 50%, at least 70%, at least 90%, or 100%. In this latter case, it can be the case that there is no longer a detectable value associated with the parameter.

The use of a substance as a medicament as described in this document can also be interpreted as the use of said substance in the manufacture of a medicament. Similarly, whenever a substance is used for treatment or as a medicament, it can also be used for the manufacture of a medicament for treatment.

The word “about” or “approximately” when used in association with a numerical value (e.g. about 10) preferably means that the value may be the given value (of 10) more or less 0.1% of the value.

The word “around” when used in association with a given temperature preferably means in a range from said given value minus 3° C. up to said given value plus 3° C., more preferably in a range from said given value minus 2° C. up to said given value plus 2° C., most preferably in a range from said given value minus 1° C. up to said given value plus 1° C.

The proposition “between” when used in association with integers refers to a range including the boundary values mentioned. For example, if n is a value between 1 and 3, n may be 1, 2 or 3. In other words, “between X and Y” is a synonym of “from X up to Y”.

The term “at least” in front of a list of numbers applies to all numbers of said list, meaning that “at least 1, 2, or 3” has the same meaning as “at least 1, at least 2, or at least 3”. This applies mutatis mutandis to terms such as “from” and “up to”. For example, “from 1, 2, or 3 up to 4” has the same meaning as “from 1 up to 4, from 2 up to 4, or from 3 up to 4”.

In the context of this invention, “represented by structure X”, “of structure X” and “with structure X” are used interchangeably.

A concentration in the context of this invention is a molar concentration, unless explicitly defined otherwise. A percentage referring to a concentration in the context of this invention is a molar percentage, unless explicitly defined otherwise. Therefore “%” means “mol %”, unless explicitly mentioned otherwise.

A concentration of a compound comprised in a bilayer is preferably defined relative to the total number of lipids in said bilayer, unless explicitly defined otherwise.

Specifically, a concentration of DPPG₂ in this application refers to the molar concentration of DPPG₂ in a bilayer, preferably wherein said bilayer is comprised in a thermosensitive liposome according to the invention, relative to the total number of lipids comprised in said bilayer.

In the context of this invention, solutions and/or compositions and/or bilayers having “essentially the same lipid composition” means that for each type of lipid, the molar concentration of said lipid differs by less than 10%, 9.5%, 9%, 8.5%, 8%, 7.5%, 7%, 6.5%, 6%, 5.5%, 5%, 4.5%, 4%, 3.5%, 3%, 2.5%, 2%, 1.5%, 1%, 0.9%, 0.8%, 0.7%, 0.6%, 0.5%, 0.4%, 0.3%, 0.2%, or 0.1% between said, solutions and/or compositions and/or bilayers. Compounds or compositions according to this invention are preferably for use in methods or uses according to this invention.

In the context of this application, the terms cell, cell line and cell culture may be used interchangeably. All of these terms also include their progeny, which is any and all subsequent generations formed by cell division. It is understood that all progeny may not be identical due to deliberate or inadvertent mutations.

In the context of this application, spherical means roughly or essentially spherical, and should not be interpreted as an absolute geometric property. Specifically, a liposome is not limited to perfectly spherical systems, but also includes roughly spherical systems.

Molar concentration of a compound in a composition, in a structure or in a (molecular) system is defined as the ratio of the number of molecules of said compound and the total number of molecules comprised in said compositions, said structure or said system.

The molar ratio between a first compound and a second compound in a composition, in a structure or in a (molecular) system is defined as the ratio between the number of molecules of said first compound comprised in said composition, said structure or said system, and the number of molecules of said second compound in said composition, said structure or said system.

An animal is preferably a mammal, more preferably selected from the group consisting of a mouse, a rat, a dog and a human, most preferably a human.

### LEGEND TO THE FIGURES

FIG. 1— Schematic representation of a thermosensitive liposome (TSL) formed by a membrane bilayer of amphiphilic phospholipid excipients encapsulating an active pharmaceutical ingredient (API).

FIG. 2— Schematic representation of the large-scale manufacturing process of DPPG₂-TSL_{30%}-DOX batches. In principle also applicable for encapsulation of other active pharmaceutical ingredients after adapting e.g. intraliposomal buffer, loading puffer, active pharmaceutical ingredient solution and process parameters.

FIG. 3— Dynamic light scattering experiments showed that the ζ-potential significantly increased for TSL with mol % DPPG₂ due to protein adsorption.

FIG. 4— ELISA for in vitro complement activation of different thermosensitive liposome (TSL) formulations in human plasma. C3a (A), Bb (B) and SC5b-9 (C) were used as readout. One-way ANOVA Dunnett's test was conducted with 5% PEG TSL as a control data set for determination of significant difference of complement activation versus standardly used TSL formulations. N=3 for each formulation tested. *=p<0.05, **=p<0.01, ***=p<0.001, ****=p<0.0001.

FIG. 5—Optimal DPPG₂ content in DPPC/DSPC/DPPG₂ 80-x:20:x (mol/mol) with x=10, 20, 30 (mol % DPPG₂-TSL). N=4 for each formulation tested.

FIG. 6— Effect of intraliposomal pH in DPPG₂-TSL_{30%}-DOX on (A) in vitro temperature-dependent DOX release in fetal calf serum (FCS), and (B) lipid decomposition during storage at 2-8° C.

FIG. 7—Effect of intraliposomal pH in DPPG₂-TSL_{30%}-DOX on in vitro temperature-dependent DOX release in fetal calf serum (FCS) during storage of formulations as frozen (−20° C., A-C) or liquid (2-8° C., D-F) dispersion.

FIG. 8—Effect of molar API:lipid ratio in DPPG₂-TSL_{30%}-DOX (intraliposomal buffer: ammonium sulfate pH 5.4) on (A) vesicle size (z average) measured by DLS, (B) PDI measured by DLS, (C) Total lipid excipient content (DPPC; DSPC, DPPG₂, without including peak areas of lipid-related decomposition products) by HPLC/CAD, (D) Total DOX content (without including peak areas of DOX-related decomposition products) by HPLC, (E) background fluorescence as indicator for DOX leakage (background fluorescence intensity increases when intraliposomal, quenched DOX is released and diluted into extraliposomal medium), and TDR profile in FCS with DOX release within 5 min of incubation at (F) 37° C., (G) 38° C., (H) 39° C., (I) 40° C.

FIG. 9—Effect of molar API:lipid ratio in DPPG₂-TSL_{30%}-DOX (intraliposomal buffer: ammonium phosphate pH 7.4) on (A) vesicle size (z average) measured by DLS, (B) PDI measured by DLS, (C) Total lipid excipient content (DPPC; DSPC, DPPG₂, without including peak areas of lipid-related decomposition products) by HPLC/CAD, (D) Total DOX content (without including peak areas of DOX-related decomposition products) by HPLC, (E) background fluorescence as indicator for DOX leakage (background fluorescence intensity increases when intraliposomal, quenched DOX is released and diluted into extraliposomal medium), and TDR profile in FCS with DOX release within 5 min of incubation at (F) 37° C., (G) 38° C., (H) 39° C., (I) 40° C.

FIG. 10— Representative of cryo-TEM images of DPPG₂-TSL_{30%}-DOX loaded with DOX using ammonium phosphate pH 7.4 buffer. Vesicle size (DLS) was 118.2 nm (PDI 0.120). Cryo-TEM was performed with a Titan EFTEM cryo 300 kV low dose mode microscope. Blot time: 4 sec, blot position: −3, grids: Quantifoil 2/2, temperature: 22° C.

FIG. 11—Effect of vesicle size & molar API:lipid ratio of DPPG₂-TSL_{30%}-DOX (intraliposomal buffer: ammonium phosphate pH 7.4) on (A) Total lipid excipient content (DPPC; DSPC, DPPG₂, without including peak areas of lipid-related decomposition products) by HPLC/CAD, (B) Total DOX content (without including peak areas of DOX-related decomposition products) by HPLC, (C) background fluorescence as indicator for DOX leakage (background fluorescence intensity increases when intraliposomal, quenched DOX is released and diluted into extraliposomal medium), and TDR profile in FCS with DOX release within 5 min of incubation at (D) 38° C.

FIG. 12— Effect of extraliposomal salt & cryoprotectant concentration on storage stability of DPPG₂-TSL_{30%}-DOX as liquid dispersion: (A) effect on vesicle size (z average), (B) effect on background fluorescence as indicator for DOX leakage (background fluorescence intensity increases when intraliposomal, quenched DOX is released and diluted into extraliposomal medium). Freezing and thawing stability of DPPG₂-TSL_{30%}-DOX: Effect of up to 6 freezing and thawing cycles (FTC) on (C) vesicle size (z average), and (D) background fluorescence as indicator for DOX leakage.

FIG. 13— Therapeutic efficacy study in Brown Norway rats implanted with a s.c. BN175 sarcoma tissue fragment on the left hind limb. Once the tumor was brought to HT (i.e. 41° C.) by lamp heating, 0.9% saline (A), non-liposomal DOX (2 mg/kg; B) or DPPG₂-TSL_{30%}-DOX batch 1 (2 mg/kg; C) were i.v. administered by tail vein catheter and heating was maintained for an hour thereafter. The Kaplan-Meier plot based on tumor volume tripling time shows a significant difference in tumor growth delay between animals treated with saline (0.9%), non-liposomal DOX and DPPG₂-TSL_{30%}-DOX batch 1 (D). Data represent n=6 per group. One-way ANOVA Tukey test was performed to determine differences between treatment groups.

FIG. 14— Rat injection scheme for investigation of pharmacokinetics (PK) and accelerated blood clearance (ABC) of carboxyfluorescein (CF)-loaded thermosensitive liposomes (TSL. At Day 0, rats were injected with a formulation of choice at a lipid dose of 5 or 75 μmol/kg followed by a direct PK experiment afterwards or the PK followed immediately after a second administration given 7, 14, 21 or 28 days later.

FIG. 15— Pharmacokinetics (PK) and accelerated blood clearance of carboxyfluorescein (CF) formulated in TSL0 (A), 5% PEG TSL (B, E), 10% PEG TSL (C) and 30% DPPG2 TSL (D, F) in Brown Norway rats. In the first set of experiments (A-C) a lipid dose of 5 μmol/kg was used. In the second set of experiments (D-F) a lipid dose of 75 μmol/kg was used. PK was performed immediately after first administration (•), or immediately after a second dose given 7 days (▪), 14 days (♦), 21 days (∘) or 28 days (∇) after the first dose. The rats in the final plots (G & H) were given a first dose of 75 μmol/kg of 5% PEG TSL (G) or 30% DPPG2 TSL (H), followed by an immediate PK experiment (•). The other data sets in these plots show CF PK when rats received the same formulation and dose (▪) or 5 μmol/kg 5% PEG TSL (♦) 7 days prior to the PK experiment. Linear or mon-exponential decay curves were fitted to determine CF half-life values (Table 2). N=3 per group.

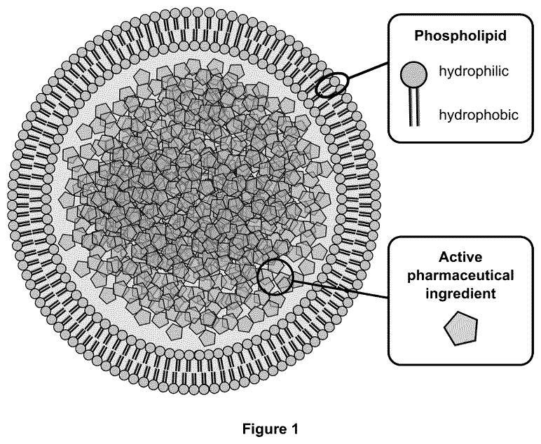

### REFERENCES

[1] M. B. Yatvin, J. N. Weinstein, W. H. Dennis, R. Blumenthal, Design of liposomes for enhanced local release of drugs by hyperthermia, Science 202 (1978) 1290-1293.
[2] D. Needham, G. Anyarambhatla, G. Kong, M. W. Dewhirst, A new temperature-sensitive liposome for use with mild hyperthermia: characterization and testing in a human tumor xenograft model, Cancer Res 60 (2000) 1197-1201.
[3] L. H. Lindner, M. E. Eichhorn, H. Eibl, N. Teichert, M. Schmitt-Sody, R. D. Issels, M. Dellian, Novel temperature-sensitive liposomes with prolonged circulation time, Clin Cancer Res 10 (2004) 2168-2178. https://doi.org/10.1158/1078-0432.CCR-03-0035.
[4] M. Hossann, M. Wiggenhorn, A. Schwerdt, K. Wachholz, N. Teichert, H. Eibl, R. D. Issels, L. H. Lindner, In vitro stability and content release properties of phosphatidylglyceroglycerol containing thermosensitive liposomes. Biochim Biophys Acta 1768 (2007) 2491-9. https://doi.org/10.1016/j.bbamem.2007.05.021
[5] S. Limmer, J. Hahn, R. Schmidt, K. Wachholz, A. Zengerle, K. Lechner, H. Eibl, R. D. Issels, M. Hossann, L. H. Lindner, Gemcitabine treatment of rat soft tissue sarcoma with phosphatidyldiglycerol-based thermosensitive liposomes, Pharm Res 31 (2014) 2276-86. https://doi.org/10.1007/s11095-014-1322-6
[6] L. Willerding, S. Limmer, M. Hossann, A. Zengerle, K. Wachholz, T. L. M. ten Hagen, G. A. Koning, R. Sroka, L. H. Lindner, M. Peller, Method of hyperthermia and tumor size influence effectiveness of doxorubicin release from thermosensitive liposomes in experimental tumors, Journal of Controlled Release 222 (2016) 47-55. https://doi.org/10.1016/j.jconre1.2015.12.004.

### EXAMPLES

The following examples are offered for illustrative purposes only, and are not intended to limit the scope of the present invention in any way.

### Example 1: Liposome Preparation and Biophysical Characterization

Thermosensitive liposomes and compositions thereof, also called thermosensitive (TSL) formulations, can be prepared by distinct preparation processes (e.g. lipid film hydration and extrusion method, ethanol injection, or other methods). Active pharmaceutical ingredient is either loaded passively or by an active process. Active remove loading can be achieved by using, for example, an ammonium gradient, an acid gradient, or an EDTA salt gradient.

TABLE 1
Examples for buffers and active pharmaceutical ingredient solutions
used for preparation of distinct DPPG₂-TSL preparations.

| | Loading | | Loading | | Storage |
|---|---|---|---|---|---|
| API | method | Intraliposomal buffer | buffer | API solution | buffer |
| HBS pH 7.4 | Passive | HBS pH 7.4 | Not needed | Not applicable | HBS pH 7.4 |
| CF | Passive | 100 mM CF pH 7.2 | Not needed | See | HBS pH 7.4 or |
| | | | | intraliposomal | 0.9% saline |
| | | | | buffer | |
| DOX (citrate) | Active | 300 mM citric acid pH | HBS pH 7.4 | 5.7 mg/ml | HBS pH 7.4 |
| | | 4 | | doxorubicin HCl | |
| DOX | Active | 240 mM (NH₄)₂SO₄ pH | HBS pH 7.4 or | 5.7 mg/ml | 10% (w/v) |
| (ammonium | | 5.4 | PBS pH 7.4 | doxorubicin HCl | trehalose, 10.5 |
| sulfate) | | | | | mM Na/K |
| | | | | | phosphate |
| DOX | Active | 300 mM (NH₄)₂HPO₄ | HBS pH 7.4 or | 5.7 mg/ml | 10% (w/v) |
| (ammonium | | pH 7.4 | PBS pH 7.4 | doxorubicin HCl | trehalose, 10.5 |
| phosphate) | | | | | mM Na/K |
| | | | | | phosphate |
| CPT-11 | Active | 300 mM (NH₄)₂SO₄ pH | HBS pH 7.8 | 20 mg/ml | HBS pH 7.4 |
| | | 7.4 | | Irinotecan HCl | |
| dFdC | Passive, | HBS pH 7.4 | HBS pH 7.4 | 38 mg/ml dFdC, | HBS pH 7.4 |
| | equilibrium | | | pH 3 or pH 6 | |
| | method | | | (adjusted by | |
| | | | | addition of 600 | |
| | | | | mM NaHCO₃ | |

Small scale preparation methods for DPPG₂-TSL with encapsulated CF or DOX are state-of-the art. In brief, all solutions were prepared with deionized and purified water from the ultrapure water system (Milli Q Advantage, Millipore) and subsequently filtered through 0.2 μm before usage. Phospholipids in the desired molar ratio (e.g. DPPC/DSPC/DPPG₂ 50:20:30 for DPPG₂-TSL_{30%}) were dissolved in chloroform/methanol 9:1 (vol/vol) using a round-bottomed flask. The solvent was evaporated under vacuum in a rotary evaporator until a thin and homogeneous lipid film was formed. The lipid film was dried for at least 1 hour at 10 mbar/70° C. to remove remaining traces of organic solvent. Hydration of the film was performed using the intended intraliposomal buffer/API solution (Table 1) at 60° C. for 30 min under shaking. The resulting lipid concentration was 50 mM. Unilamellar vesicles were obtained by 10 times extrusion at 60° C. with maximum 20 bar N₂ pressure through two polycarbonate filters of desired pore size (e.g. 200 nm, Whatman, GE Healthcare Europe GmbH, Freiburg, Germany) using a thermobarrel extruder (LIPEX™, Northern Lipids Inc. Burnaby, BC, Canada). Subsequently, the dispersion was cooled to 2-8° C. and buffer was exchanged to loading buffer (Table 1, if active drug or equilibrium loading is performed) or storage buffer (Table 1, if passive loading was performed) applying PD10 columns (GE Healthcare). If the API is loaded by an active or equilibrium method, the following steps have been performed. The desired loading conditions (e.g. molar drug/lipid ratio and phospholipid concentration) were obtained by mixing the liposomal dispersion with loading buffer and the API solution (Table 1). The dispersion was heated under shaking (e.g. 37° C. up to 60 min for DOX, 37° C. for 45 min for CPT-11, 60° C. for 30 min for dFdC) in an Eppendorf Thermomixer comfort with 50 ml thermoblock. Traces of unencapsulated API were removed by centrifugation at 75,000×g (Beckman Coulter Avanti J-26 XP with a JA-25.50 rotor). The supernatant was discarded and the pellet was carefully resuspended with storage buffer (Table 1).

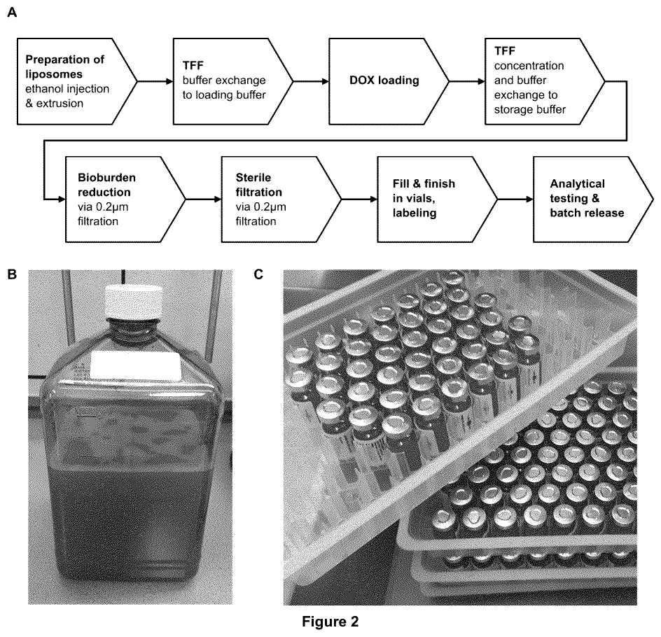

For large-scale preparation of DPPG₂-TSL, an adapted method was applied using appropriate equipment to handle larger dispersion volumes, while the standard steps of preparation have not been changed. The applied process is schematically shown in FIG. 2. In contrast to the small-scale process, liposomes have been produced by ethanol injection instead of lipid film hydration and buffer exchange was performed with tangential flow filtration (TFF) instead of chromatography or centrifugation, respectively.

Characterization of TSL preparations is known in the art. The hydrodynamic diameter (z average), size intensity distribution plot and zeta potential were determined by dynamic light scattering (DLS, Zetasizer Nano ZS, Malvern Instruments, Worcestershire, United Kingdom). The instrument was calibrated with a Nanosphere™ size standard (125 nm, Thermo Fisher Scientific, Waltham, MA, USA). Phospholipid composition was quantitatively measured with TLC. TLC plates were developed with chloroform/methanol/acetic acid (97.5%)/water 100/60/10/5 (vol/vol) to achieve the separation of phosphatidylcholines (DPPC, DSPC) from DPPG₂, lyso-phosphatidylcholine (Lyso-PC) and lyso-phosphatidyldigylcerol (Lyso-PG₂). A lipid standard containing DPPG₂, DPPC and 1-palmitoyl-sn-glycero-3-phosphocholine (P-Lyso-PC) was applied in every TLC run to check the separation quality. Phospholipid concentration was measured by phosphate analysis using a 1 g/l phosphate solution (Merck KGaA, Darmstadt, Germany) as reference standard. In vitro temperature-dependent API release (TDR) have been analyzed in fetal calf serum (FCS) as described previously.

Purity and concentration of DOX, CPT-11, dFdC, and the lipid excipients have been quantified with HPLC. Free fatty acids have been quantified with the enzymatic endpoint method using the NEFA kit from DiaSys Diagnostic Systems GmbH (Holzheim, Germany) according their standard protocol.

### Example 2: Increasing DPPG₂ Content in TSL Reduce Complement Activation

Investigated TSL Formulations

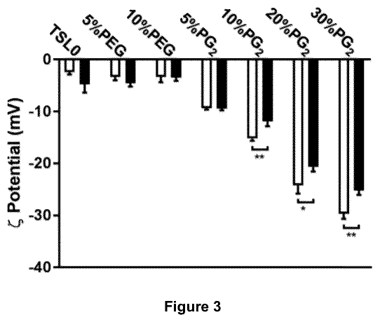

Multiple TSL formulations differing in lipid composition have been described until now, but it is unclear which one might be most optimal in a clinical setting. Sufficient circulation half-life of the TSL during HT treatment is only one important factor for optimal drug delivery to solid tumors. Of high importance is that these formulations can be intravenously applied to patients without severe toxicity like CARPA. These hypersensitive reactions can give serious complications during infusion of the particles which may require intervention or preventive medication. Therefore, several TSL formulations have been prepared to investigate the effect of DSPE-PEG2000 and DPPG₂ on complement activation. Lipid compositions were DPPC/DSPC/DSPE-PEG2000 80:20-x:x (mol/mol) x=5, 10, 20, 30 (PEG-TSL_{x %}) and DPPC/DSPC/DPPG₂ 80-x:20:x (mol/mol) x=5, 10, 20, 30 (DPPG₂-TSL_{x %}). A TSL formulation without surface modification composed of DPPC/DSPC 80:20 (mol/mol) (TSL_{0%}) served as control. PEG-TSL_{20%} and PEG-TSL_{30%} showed no adequate dispersion stability and were excluded from the study. Vesicle size was between 100 to 150 nm with a PDI <0.10 for all remaining PEG-TSL and DPPG₂-TSL. DPPG₂-TSL had a negative surface charge which became more dominant with increasing mol % of DPPG₂ (FIG. 3C).

Complement Activation

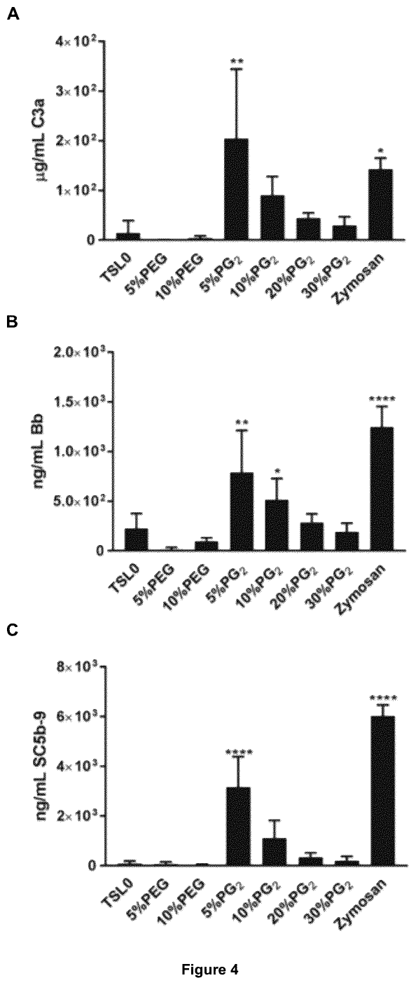

Complement activation by TSL formulations was evaluated with C3a, Bb and SC5b-9 ELISA-kits (Quidel, San Diego, CA) on TSL-incubated human plasma. 15 μL TSL (25 mM) with encapsulated HBS pH 7.4 were added to 135 μL of human citrate-anticoagulated plasma and incubated at 37° C., 750 rpm for 30 min in a thermoshaker. The incubation was terminated by diluting the samples in C3a, Bb or SC5b-9 sample diluent supplied in corresponding kits. Samples were loaded onto ELISA wells and assay was carried out by manufacturer's instructions. 15 μL of 10 mg/mL Zymosan A in 0.9% saline was used as a positive control for the assay. After incubation of serum with Zymosan, the sample was centrifuged at 800×g for 10 min and the supernatant was diluted in sample diluent similarly as described above. Plates were measured for absorbance at 450 nm by MRX Microplate Reader (Dynatech Laboratories, Alexandria, VA). TSL_{0%} and PEG-TSL showed no complement activation, whereas DPPG₂-TSL_{5%} incubation induced a significant increase in C3a, Bb and SC5b-9 levels (FIG. 4). Increasing the mol % of DPPG₂ to 30 mol % reduced the nanoparticle induced complement activation significantly.

It is known, that anionic nanoparticles are potent complement activators and therefore might induce toxicity after i.v. application to patients. Incorporation of higher amounts of anionic DPPG₂ into TSL formulation however surprisingly decreased complement activation and this is in clear contrast to CARPA studies on other negatively charged nanoparticles which describes the opposite and has led to an understanding that negatively charged nanoparticles have to be carefully investigated before clinical use and that not just the negative surface charge as such, but also the amount of it and the specific characteristics of the lipid head group play a key role in CARPA. The low complement activation in human plasma for DPPG₂-TSL_{20%} and DPPG₂-TSL_{30%} is unique and could be an indication that these formulations have a beneficial toxicity profile over other negatively charged formulations which are currently in clinical practice.

Results from a GLP compliant toxicity study with DPPG₂-TSL_{30%} (batch 3, Table 3) in dogs supported the in vitro findings of reduced complement activation. 10 dogs (5 male/5 female) received i.v. infusions of DPPG₂-TSL_{30%} (5.6 mg DPPG₂ per kg bodyweight per dose) every three weeks for 12 weeks. No signs of anaphylactic reactions were recorded. This is in contrast to LTSL, were premedication of the dogs was necessary to suppress anaphylactic reactions.

### Example 3: DPPG₂ Content in TSL Avoids Occurrence of Accelerated Blood Clearance

Investigated TSL Formulations

Preparation and composition of investigated liposomal formulations is summarized in Example 2.

Experimental Procedure

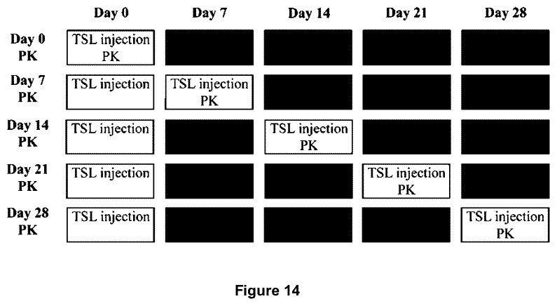

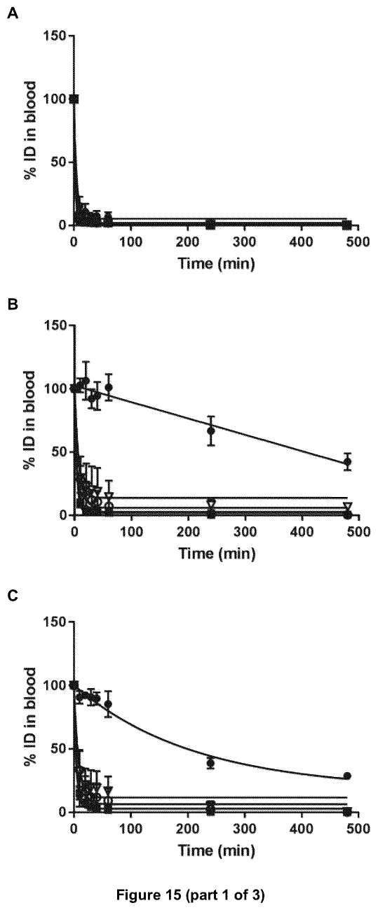

Pharmacokinetic (PK) experiments were performed as described in FIG. 14. 1 mL CF-loaded TSL was injected via penile vein in BN rats at a 5 μmol/kg or 75 μmol/kg lipid dose. 180 μL blood samples were collected in citrate tubes by tail vein cut at t=0 min (prior to TSL administration), 10 min, 20 min, 30 min, 40 min, 60 min, 4 hours and 8 hours and centrifuged at 1.300× g for 10 min to obtain plasma. After PK experiments, the rats were euthanized by an intracardial 300 mg/kg pentobarbital injection and for the groups of interest, liver, spleen and kidneys were excised and stored at −20° C. for later use. Plasma samples were analyzed for CF content by fluorometry after incubation in 10% Triton X-100 at 45° C. for 15 min to ensure full CF release. The percentage injected dose (% ID) in the plasma samples was calculated by comparison of the sample signal to the signal of a 100% sample which represented the maximum expected CF content in the total blood volume of the rat, calculated from its body weight. CF fluorescence was measured in 0.9% NaCl/10 mM Tris buffer pH 8.0 (440:80, v:v) at 493 nm excitation and 513 nm emission.

Accelerated Blood Clearance

Various published studies have shown that antibody responses to PEG (anti-PEG IgM) after the first administration of PEGylated liposomes can greatly reduce the pharmacokinetic profile of a following administration, a phenomenon which is described as accelerated blood clearance (ABC).

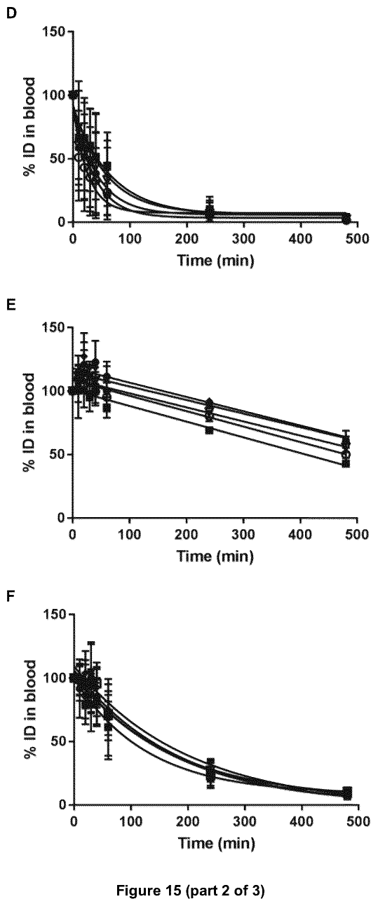

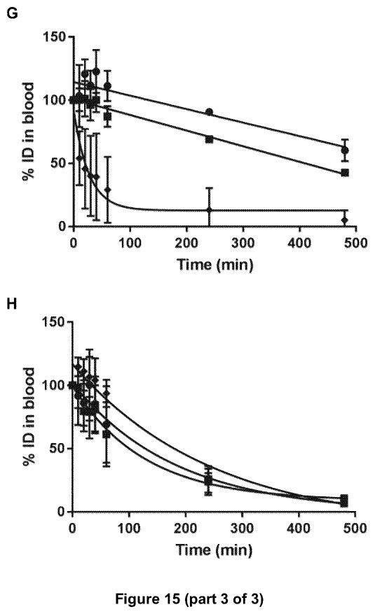

CF measurements in plasma of TSL0-injected rats showed that the absence of a surface modification causes a negligible circulation time of the encapsulated fluorophore with a circulation half-life (t_{1/2}) of several minutes (FIG. 15A; Table 2). CF encapsulated in 5% PEG TSL and 10% PEG TSL had a substantially longer t_{1/2} with 332±124 min and 155±44 min, respectively (FIGS. 15B & 15C; Table 3). However, if an additional administration of pegylated TSL preceded the PK experiment by 7-28 days, the t_{1/2} of CF decreased to less than 10 min and this persisted for the entire 28 day window. Our findings show that the ABC effect is very effective and persistent in negatively affecting the PK profile for PEGylated TSL formulations, even if anti-PEG IgM levels are low. The DPPG₂ surface coating proved less efficient than PEG in improving CF t_{1/2} as the 30% DPPG₂ TSL had a CF t_{1/2} of 29±31 min (FIG. 15D; Table 2). However, the CF t_{1/2} did not change because of an earlier 30% DPPG₂ TSL administration. When the same experiment was repeated at a 75 μmol/kg lipid dose, the CF t_{1/2} increased for 5% PEG TSL and 30% DPPG₂ TSL to 611±93 min and 138±76 min, respectively (FIGS. 15E & 15F; Table 2). The CF t_{1/2} was not altered for 30% DPPG₂ TSL if pre-dosed with the same formulation at 75 μmol/kg, which was a similar observation as the previously mentioned results at 5 μmol/kg. On the contrary, the increase in dose greatly reduced the ABC effect for 5% PEG TSL. If rats were first given an injection of 5 μmol/kg 5% PEG TSL, followed by a PK experiment one week later with 75 μmol/kg 5% PEG TSL or 30% DPPG₂ TSL, the CF t_{1/2} were 41±53 min and 174±53 min, respectively (FIGS. 15G & 15H; Table 2). The inverse correlation between liposome dose and ABC is known for long circulating liposomes with very high membrane stability. For TSL formulations however, drug leakage is perhaps the most important factor to consider, also when administered at a higher dose. As earlier described in this section, even a relatively low amount of anti-PEG IgM can be particularly successful in destabilizing a TSL membrane and largely diminish the t_{1/2} of the encapsulated compound.

TABLE 2
Circulation half-life of carboxyfluorescein encapsulated in different TSL formulations.

| | t_{1/2} | t_{1/2} | t_{1/2} | t_{1/2} | t_{1/2} |
|---|---|---|---|---|---|
| | Day 0 | Day 7 | Day 14 | Day 21 | Day 28 |
| | (min) | (min) | (min) | (min) | (min) |
| TSL0 (5 μmol/kg) | 3 ± 1 | 2 ± 0 | 2 ± 0 | 2 ± 0 | 2 ± 1 |
| 5% PEG TSL (5 μmol/kg) | 332 ± 124 | 3 ± 0 | 3 ± 0 | 6 ±2 | 8 ± 8 |
| 10% PEG TSL (5 μmol/kg) | 155 ± 44 | 3 ± 1 | 3 ± 1 | 7 ± 3 | 7 ± 3 |
| 30% PG₂ TSL (5 μmol/kg) | 29 ± 31 | 53 ± 31 | 52 ± 46 | 21 ± 23 | 42 ± 57 |
| 5% PEG TSL (75 μmol/kg) | 611 ± 93 | 412 ± 13 | 602 ± 7 | 486 ± 43 | 537 ± 50 |
| 30% DPPG₂ TSL (75 μmol/kg) | 138 ± 76 | 106 ± 91 | 147 ± 69 | 134 ± 57 | 120 ± 39 |
| 5 (5% PEG)-75 (5% PEG) | | 41 ± 53 | | | |
| 5 (5% PEG)-75 (30% DPPG₂) | | 174 ± 53 | | | |

Therefore, additional rat PK studies were performed with PEG-TSL₅%, PEG-TSL_{10%}, or DPPG₂-TSL_{30%} with encapsulated CF for evaluating PK parameters. PEG-TSL showed ABC as taught by the state-of-the art, whereas DPPG₂-TSL_{30%} administration in rats showed no ABC. Since the absence of ABC for nanoparticles coated with polyglycerol moieties was shown before, it is reasonable to assume, that coating with oligoglycerols like e.g. DPPG₂ also prevents ABC. Another important factor to take into account is the presence of anti-PEG antibodies in treatment-naïve patients due to frequent exposure to PEG in commonly used products which could already impair the first treatment round of a pegylated TSL. Because of these reasons it is clinically relevant to investigate TSL formulations that do not use PEGylation, therefore making DPPG₂-TSL an interesting candidate for clinical development.

### CONCLUSION

The results of Example 2 show, that the known complement activation of anionic nanoparticles can be suppressed by using >10 mol % DPPG₂ in TSL formulations. As shown in Example 2 and Example 3, together with the prolonged circulation half-life, the absence of ABC phenomenon after repeated injection and the absence of signs of anaphylactic reaction in dogs, qualifies API-containing DPPG₂-TSL as promising candidate for preclinical development.

In conclusion, attractive results were obtained for thermosensitive liposomes according to the invention wherein the concentration of DPPG₂ in the bilayer is at least 15 mol %. Even more attracting results were obtained for thermosensitive liposomes according to the invention wherein the concentration of DPPG₂ in the bilayer is from 15 mol % up to 35 mol %.

### Example 4: Increasing DPPG₂ Content in TSL Result in Superior API Accumulation at Target Site

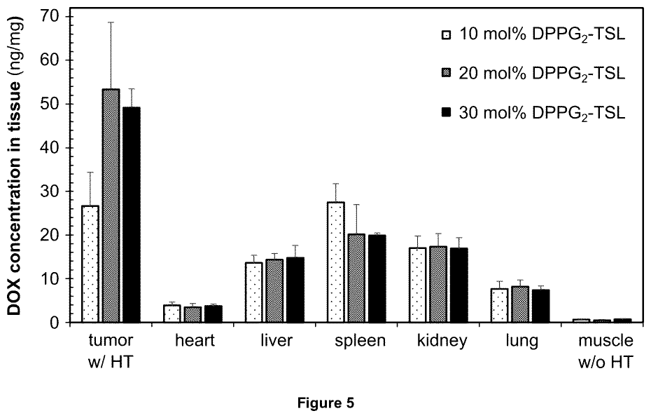

Optimal DPPG₂ content in DPPC/DSPC/DPPG₂ 80-x:20:x (mol/mol) with x=10, 20, 30 (DPPG₂-TSL_{x %}) to achieve therapeutic efficacy was investigated by a biodistribution (BD) and doxorubicin (DOX) tumor enrichment study in Brown Norway rats. DSPC content was set to 20 mol % in all batches to achieve a comparable fatty acid composition in the TSL membrane to be palmitic acid to stearic acid 80:20 (mol/mol). Three independent DPPG₂-TSL formulations differing in DPPG₂-content (10, 20, 30 mol %) have been prepared using 300 mM citrate pH 4 for active loading of DOX. Z average was 125.1 nm, 120.5 nm, and 114.8 nm for DPPG₂-TSL_{10%}, DPPG₂-TSL_{20%}, and DPPG₂-TSL_{30%}, respectively. PDI was <0.07 for all three batches. As to be expected from DPPG₂ content, zeta potential decreased from −13.6 mV for DPPG₂-TSL_{10%}, −22.7 mV for DPPG₂-TSL_{20%}, to −31.1 mV for DPPG₂-TSL_{30%}. Lysolipid content was −1 mol % for each batch, indicating a comparable decomposition of the liposomes during manufacturing. Final molar DOX:lipid ratio was 0.094, 0.104, and 0.106 for DPPG₂-TSL_{10%}, DPPG₂-TSL_{20%}, and DPPG₂-TSL_{30%}, respectively. In vitro DOX release in FCS during 5 min at 41° C. increased with increasing DPPG₂-content: 16.8%, 63.7% 93.2% for DPPG₂-TSL_{10%}, DPPG₂-TSL_{20%}, and DPPG₂-TSL_{30%}, respectively. This observation is consistent to the published effect of DPPG₂ on content release rate. The PK profile in Brown Norway rats in the first 60 mins was comparable for all three formulations showing a monoexponential clearance as known from the state-of-the-art (WO9730058, WO2014202680). Next, a BD study in BN175 tumor-bearing rats, receiving 2 mg/kg DOX i.v. in combination with 60 min of lamp hyperthermia (HT) of the tumor was performed (FIG. 5). Tumor volumes were −1 cm 3. DPPG₂-TSL_{x %} were i.v. injected in the tail vein after a target temperature of 41° C. was reached in the tumor. At the end of HT, a blood sample was taken with cardiac puncture before the animals were euthanized. After cardiac perfusion, tissue samples were collected. DOX content was quantified by HPLC. DPPG₂-TSL_{20%} and DPPG₂-TSL_{30%} achieved a markedly higher tumor drug deposition than the DPPG₂-TSL_{10%}. There was no difference in DOX content in the other tissues except for spleen, where DOX content was higher for DPPG₂-TSL_{10%}.

Conclusion: The results show, that neither long-circulating properties of the carrier (as taught e.g. by WO02064116 for e.g. DPPG₂-TSL_{10%}) alone, nor sole in vitro drug release properties are conclusive to define the optimal lipid composition of DPPG₂-TSL that reach highest drug concentrations by heat-triggered API release in heated tumors in vivo. The BD results indicate that even slower releasing DPPG₂-TSL_{20%} reach comparable DOX levels as DPPG₂-TSL_{30%}. Long-circulating DPPG₂-TSL_{10%} showed notably less API accumulation at the target site.

In conclusion, attractive results were obtained for thermosensitive liposomes according to the invention wherein the concentration of DPPG₂ in the bilayer is at least 15 mol %. Even more attracting results were obtained for thermosensitive liposomes according to the invention wherein the concentration of DPPG₂ in the bilayer is from 15 mol % up to 35 mol %.

### Example 5: Intraliposomal pH Affects Phospholipid Excipient & API Stability of DPPG₂-TSL-API

The effect of intraliposomal buffer pH on phospholipid excipient & API stability of DPPG₂-TSL-API was evaluated.

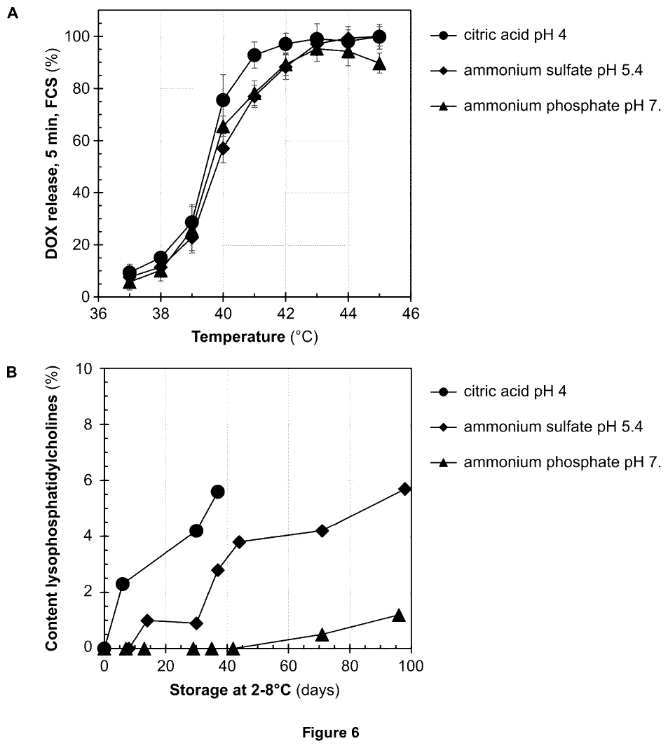

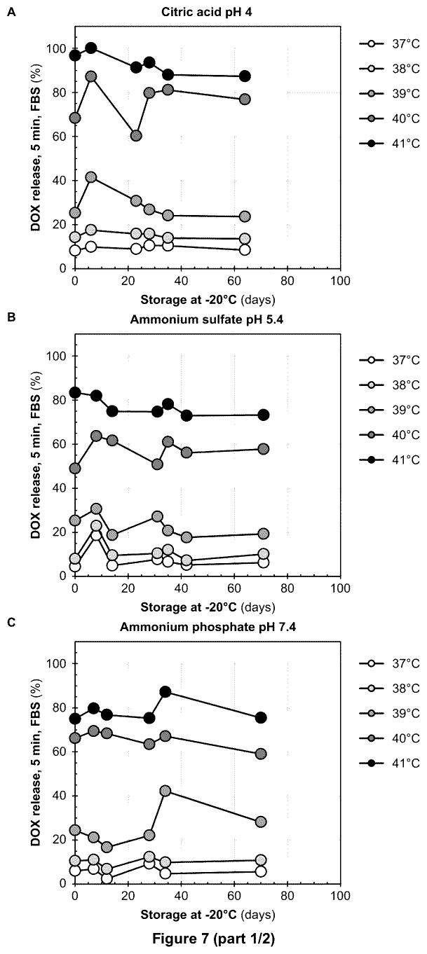

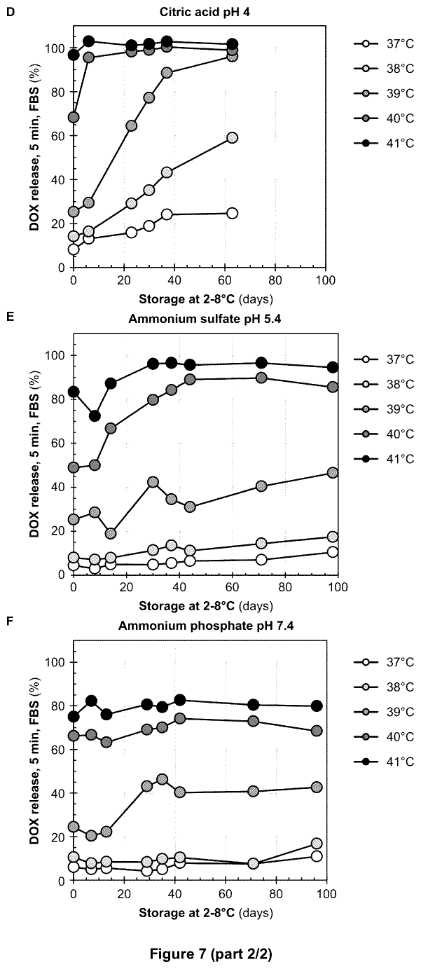

DPPG₂-TSL_{30%}-DOX have been prepared according to the small scale method described in example 1 differing in the pH value of the intraliposomal buffer used for active API loading (citric acid pH 4, ammonium sulfate pH 5.4, and ammonium phosphate pH 7.4). The formulations have been stored under liquid (2-8° C.) and frozen (−20° C.) conditions and biophysical characteristics were tested periodically. Intraliposomal pH showed no effect on the TDR profile for freshly prepared DPPG₂-TSL_{30%}-DOX (FIG. 6A). When stored in liquid condition at 2-8° C., DPPG₂-TSL_{30%}-DOX using an intraliposomal buffer of pH 4 showed rapid lipid excipient decomposition with more than 5% of lysophosphatidylcholines present after 37 days (FIG. 6B). In contrast to cholesterol-containing liposomes, where up to 15% of lipid decomposition products did not negatively affect membrane permeability, even lesser amounts of lysophosphatidylcholines (up to −5%, FIG. 6B) had a notable negative effect on the TDR, with impaired API retention at 37° C. and notable increased API release at temperatures 38° C. with every day of storage at 2-8° C. (FIG. 7D). DPPG₂-TSL_{30%}-DOX using an intraliposomal buffer of pH 5.4, showed a less rapid decomposition, but hydrolysis rate increased after 14 days and resulted in more than 5% lysophosphatidylcholines after 98 days (FIG. 6B). The TDR profile remained constant for at least 14 days of storage at 2-8° C., but changed thereafter (FIG. 7E). DPPG₂-TSL_{30%}-DOX using an intraliposomal buffer of pH 7.4 was the most stable formulation with detectable lysophosphatidylcholine content after 42 days of storage (FIG. 6B). The TDR profile remained constant for at least 14 days of storage at 2-8° C., but changed thereafter (FIG. 7D). The change in the TDR profile was less pronounced compared to DPPG₂-TSL_{30%}-DOX using an intraliposomal buffer of pH 5.4.

Preparing DPPG₂-TSL_{30%}-DOX with an intraliposomal buffer with a pH value between 6.4 to 7.4 had no effect on the stability of the TSL formulations (data not shown).

The pH value of intraliposomal buffers was also tested for DPPG₂-TSL_{30%}-dFdC. By adjusting pH of the dFdC solution to 6-6.5 before loading, no lysolipids were detectable after batch production. The change in the pH resulted also in a change in the TDR profile, with less release at 41° C. and 42° C. (22.3±10.5% at 42° C. for the formulation with pH 6 compared to 45.9±17.6% for the formulation with pH 3). The change in pH resulted in a formulation store-able at 2-8° C., whereas the published formulation had to be stored at −20° C. The formulation with pH 3 showed already more than 10% of lipid degradation products after 12 weeks storage at 2-8° C. (28.16±1.95% free fatty acids and 14.1±5.1% lysolipids), whereas for the formulation with pH 6 no change was observed in the analyzed 24 weeks. The API leakage during storage also increased already after 12 weeks for the formulation with pH 3, whereas no leakage was detectable for the pH 6 formulation.

Conclusion: Intraliposomal pH in the range of 4 to 7.4 did not affect the TDR of DPPG₂-TSL_{30%}, but significantly influenced the lipid excipient stability and TDR during storage. With increasing amounts of lipid-related impurities, the TDR profile is altered and therefore, the formulation cannot be used in treatment of patients anymore. Usage of intraliposomal pH in the range between 5.4 to 7.4 ensured phospholipid stability for at least 1 week as liquid to ensure no negative effect on TDR during production. Using of an intraliposomal pH in the range between 6.0 to 7.4 ensured phospholipid stability for at least 4 weeks as liquid to ensure no negative effect on TDR during production.

In conclusion, attractive results were obtained for thermosensitive liposomes according to the invention wherein the pH of the intraliposomal buffer is from 5.0 up to 8.0. Even more attracting results were obtained for thermosensitive liposomes according to the invention wherein the pH of the intraliposomal buffer is from 6.0 up to 8.0.

### Example 6: Loading Buffer Salt Concentration Accelerates API Loading

Accelerated temperatures during distinct manufacturing process steps can yield to decomposition of lipid excipients or API. Since loading of APIs into DPPG₂-TSL is performed at 38° C. for DOX or 60° C. for dFdC, the influence of incubation time and salt concentration in the loading buffer was evaluated.

DPPG₂-TSL_{30%} have been prepared according to the large-scale method described in example 1. Ammonium phosphate pH 7.4 was used as intraliposomal buffer during liposome manufacturing. The buffer was exchanged to distinct loading buffers differing in salt concentration. Buffer A: physiological PBS pH 7.4 without trehalose (300 mOsmol/kg). Buffer B: 4.1% (wt/v) trehalose, 10.5 mM Na/K phosphate, 66 mM saline (294 mOsmol/kg). Buffer C: 8.9% (wt/v) trehalose, 10.5 mM Na/K phosphate, 66 mM saline (294 mOsmol/kg). DOX loading was performed with an intended molar API:lipid ratio of 0.08 at 37-38° C. DOX loading was followed with fluorescence spectroscopy. At distinct time points 20 μl samples have been diluted with 3 mL HBS pH 7.4. After the fluorescence intensity of the sample is 20% of the initial fluorescence intensity without incubation and remained constant, the loading is finished. Loading in buffer A, buffer B, and buffer C was finished after 20 min, 45 min, and 90 min, respectively, indicating that API loading should be performed in physiological buffers containing high salt concentrations and no cryoprotectant.

Next, DPPG₂-TSL_{30%} have been prepared according to the large-scale method described in example 1. Ammonium phosphate pH 7.4 was used as intraliposomal buffer during liposome manufacturing and buffer A was used as loading buffer. DOX loading was performed with an intended molar API:lipid ratio of 0.08 at 37-38° C. for 30 min and 90 min. Total API-related impurity content in the batch dependent on incubation time, with <0.10 Area % and 4.1 Area % for 30 min and 90 min, respectively. Total lipid-related impurity content in the batch also dependent on incubation time, with <0.10 Area % and 0.13 Area % for 30 min and 90 min, respectively.

In conclusion, attractive results were obtained for thermosensitive liposomes according to the invention wherein said thermosensitive liposomes have been prepared using a loading buffer with a salt concentration of at least 66 mM and osmolarity of at least 250 mOsmol/kg. Even more attracting results were obtained for thermosensitive liposomes according to the invention wherein said thermosensitive liposomes have been prepared using a loading buffer with a salt concentration of at least 66 mM and osmolarity from 250 mOsmol/kg up to 350 mOsmol/kg.

### Example 7: API:Lipid Ratio Affects Stability of DPPG₂-TSL-API

DPPG₂-TSL_{30%}-DOX have been prepared according to the large-scale method described in example 1 differing in molar API:lipid ratio from 0.06 to 0.13. Either ammonium sulfate pH 5.4 or ammonium phosphate pH 7.4 were used as intraliposomal buffer, respectively. After production, the batches were stored at 5±3° C. for up to 15 weeks and biophysical characterization was performed, investigating vesicle size, PDI, total lipid excipient content (DPPC, DSPC, DPPG₂, without including peak areas of lipid-related decomposition products) by HPLC/CAD, total DOX (without including peak areas of DOX-related decomposition products) by HPLC, background fluorescence as indicator for DOX leakage, and TDR profile in FCS.

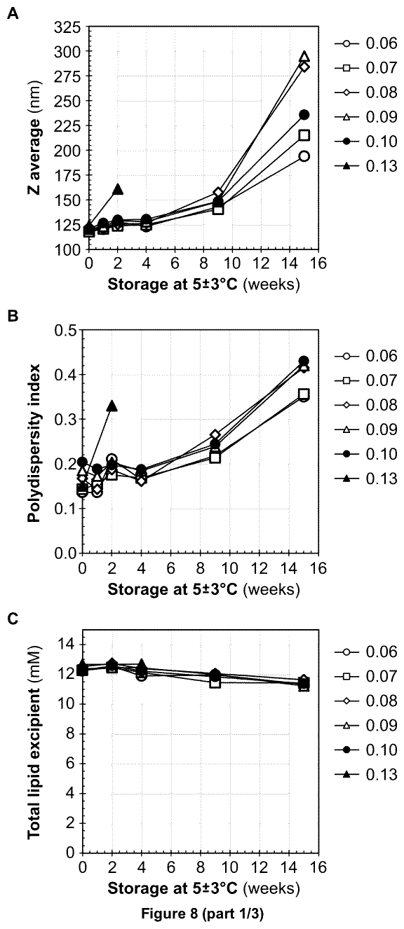

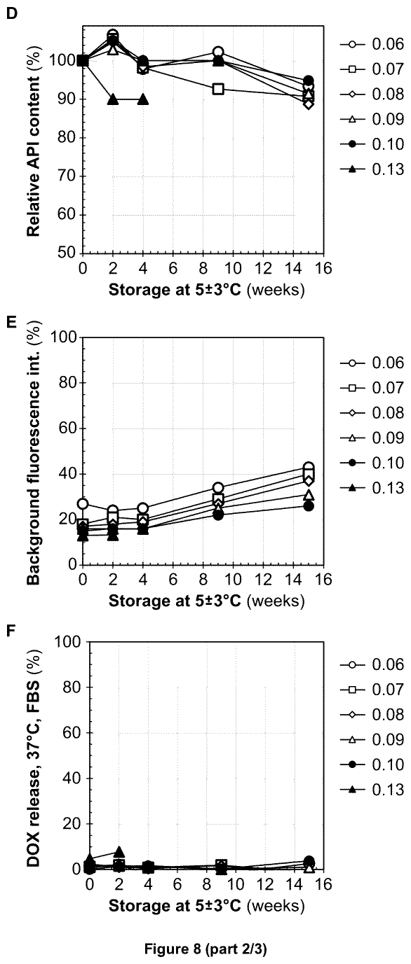

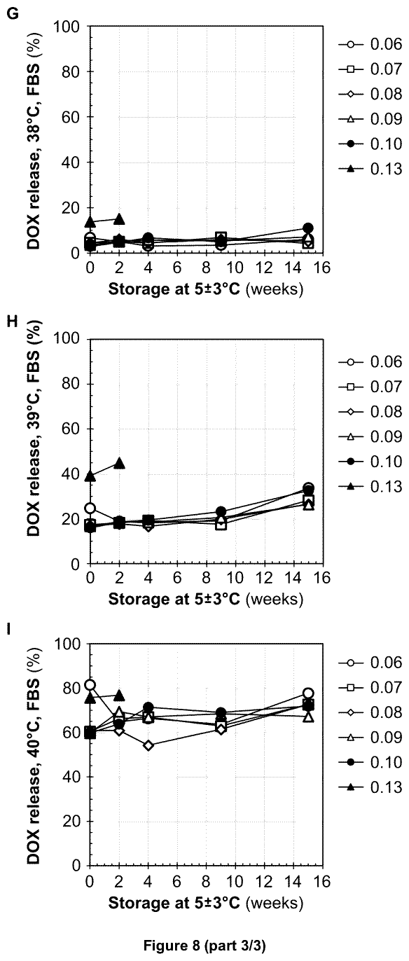

DPPG₂-TSL_{30%}-DOX (ammonium sulfate, pH 5.4) batches showed a comparable change in analytical characteristics, when molar API:lipid ratio was 0.10 (FIG. 8). In contrast, a molar API:lipid ratio of 0.13 resulted in decreased store-ability, visible in a notable increase in vesicle size (FIG. 8A), PDI (FIG. 8B), carrier stability in FCS (DOX release at 37° C., FIG. 8F), and surprisingly also a much faster decomposition of the API (FIG. 8D) after 2 weeks at 5±3° C. There was no difference in lipid decomposition (FIG. 8C) or API leakage (FIG. 8E) detectable.

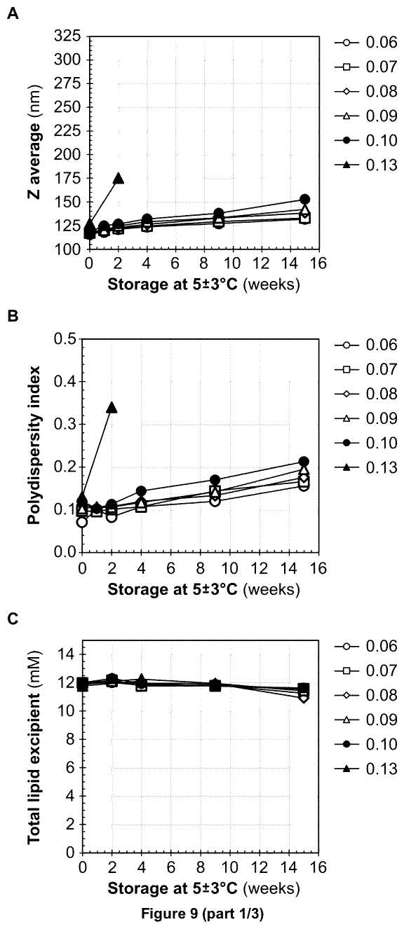

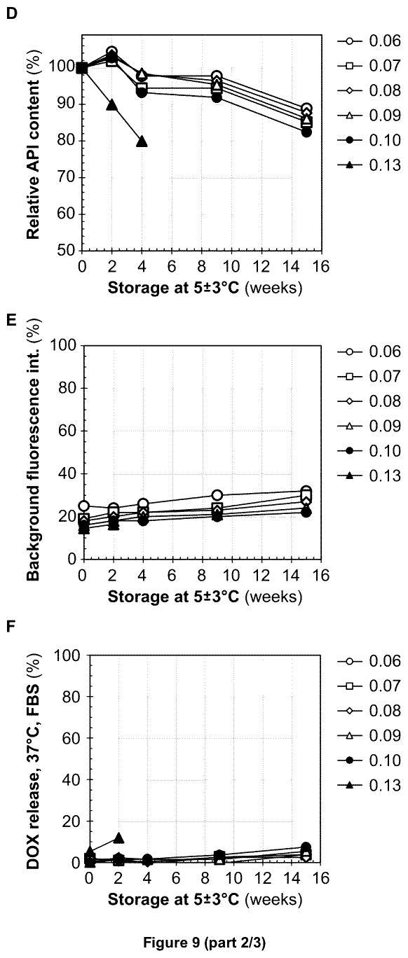

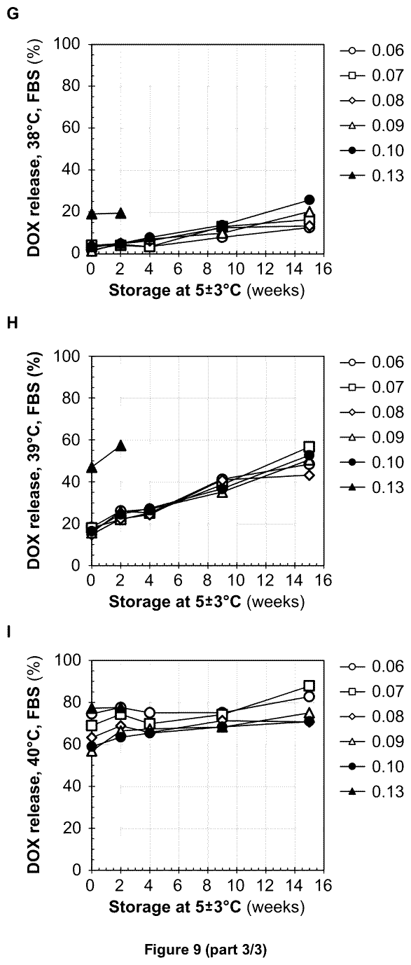

DPPG₂-TSL_{30%}-DOX (ammonium phosphate, pH 7.4) batches showed a comparable change in analytical characteristics, when molar API:lipid ratio was 0.09 (FIG. 9). In contrast, a molar API:lipid ratio of 0.13 resulted in decreased store-ability, visible in a notable increase in vesicle size (FIG. 9A), PDI (FIG. 9B), carrier stability in FCS (DOX release at 37° C., FIG. 9F), and surprisingly also a much faster decomposition of the API (FIG. 9D) after 2 weeks at 5±3° C. There was no difference in lipid decomposition (FIG. 9C) or API leakage (FIG. 9E) detectable. A tendency for destabilization was seen for the batch with a molar API:lipid ratio of 0.10.

When comparing batches of DPPG₂-TSL_{30%}-DOX (ammonium phosphate, pH 7.4) with DPPG₂-TSL_{30%}-DOX (ammonium sulfate, pH 5.4) with a molar API:lipid ratio between 0.06 and 0.10, the latter showed a faster phospholipid excipient decomposition and a slower API decomposition. Total lipid excipient content for ammonium phosphate and ammonium sulfate batches was 94.9±2.0% and 92.4±1.0% of the initial content after 15 weeks at 5±3° C., respectively. Total API content for ammonium phosphate and ammonium sulfate batches was 86.1±2.5% and 91.8±2.4% of the initial content after 15 weeks at 5±3° C., respectively. Surprisingly, these DPPG₂-TSL_{30%}-DOX (ammonium phosphate, pH 7.4) batches were more prone to a change in the TDR profile during storage compared to DPPG₂-TSL_{30%}-DOX (ammonium sulfate, pH 5.4) after 4 weeks at 5±3° C. (FIG. 8 F-H and FIG. 9 F-H) which might be rather a result of presence of API-related impurities due to API decomposition than lipid-related impurities.

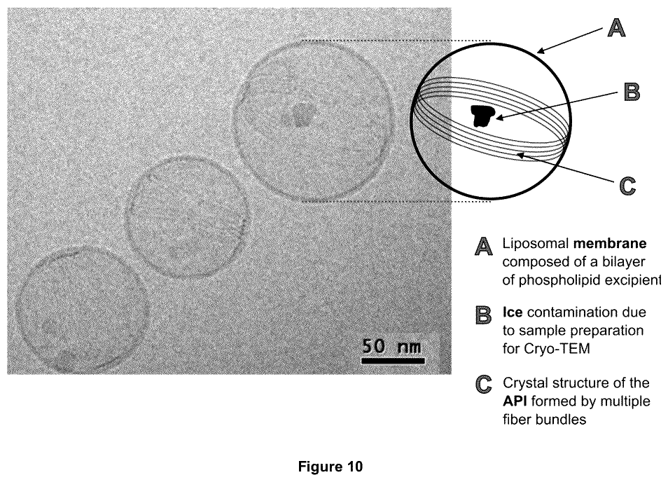

Additionally, batches have been analyzed by cryo-TEM, revealing the formation of a ring like API crystal structures composed of fibers bending around the intraliposomal lipid excipient bilayer (FIG. 10).

DPPG₂-TSL_{30%}-CPT-11 have been prepared according to the small scale method described in example 1 differing in the molar API:lipid ratio from 0.078-0.313. Ammonium sulfate pH 7.4 was used as intraliposomal buffer. After preparation the API release at 37° C. in 1 h was analyzed. It was shown, that with increasing API:lipid ratio the amount of CPT-11 released decreased. A threshold molar API:lipid ratio to have a sufficient stability at 37° C. was identified to be between 0.174 (mol/mol) and 0.21 (mol/mol), with a release of 23.4±0.6% and 10.2±0.2% in FBS, respectively. API:lipid ratios higher than 0.21 resulted in no more improvement with release at 37° C. of 12.5±3.5%.

DPPG₂-TSL_{30%}-CPT-11 batches with different API:lipid ratios (0.08 to 0.21) were prepared and stored up to 4 weeks at 2-8° C. Lysolipids, which are degradation products of lipids, were not detectable. No change in z-average and PDI as well as in the temperature dependent CPT-11 release was detectable. To evaluate if the analyzed parameters were suitable for determination of storage stability the batches were additionally stored at RT. 4.3-5.3% of lysolipids have been detected after 3 weeks storage at RT. In the temperature-dependent CPT-11 release the amount of released drug at 40° C. was increased, indicating decomposition. E.g. for DPPG₂-TSL_{30%}-CPT-11 (API:lipid ratio 0.174) four weeks' storage at 2-8° C. unaltered the amount of release at 40° C. in FBS during 5 min to be 48.9±2.5%. However, after one additional week storage at RT the release was 69.7±1.0%.

Conclusion: Intraliposomal API:lipid ratio is affecting the store-ability of DPPG₂-TSL_{30%}. The optimal API:lipid ratio strongly depends on the loaded API. Moreover, in combination with the specified API:lipid ratio, a more neutral pH used as intraliposomal buffer is preferred, because lipid excipient decomposition at 2-8° C. is slower than with a more acidic pH, despite the fact that API decomposition might be slightly faster.

In conclusion, attractive results were obtained for a thermosensitive liposome according to the invention comprising doxorubicin, a doxorubicin derivative or a pharmaceutically acceptable salt thereof as an active pharmaceutical ingredient, wherein the pH of the intraliposomal buffer is from 5.0 up to 8.0 and the molar ratio between doxorubicin, said doxorubicin derivative or said pharmaceutically acceptable salt thereof and the lipids comprised in the bilayer is from 0.06 up to 0.10, within one week avoiding freezing. Even more attractive results were obtained for a thermosensitive liposome according to the invention comprising doxorubicin, a doxorubicin derivative or a pharmaceutically acceptable salt thereof as an active pharmaceutical ingredient, wherein the pH of the intraliposomal buffer is from 6.0 up to 8.0 and the molar ratio between doxorubicin, said doxorubicin derivative or said pharmaceutically acceptable salt thereof and the lipids comprised in the bilayer is from 0.07 up to 0.09, within one week avoiding freezing.

In conclusion, attractive results were obtained for a thermosensitive liposome according to the invention comprising irinotecan, an irinotecan derivative or a pharmaceutically acceptable salt thereof as an active pharmaceutical ingredient, wherein the pH of the intraliposomal buffer is from 5.0 up to 8.0 and the molar ratio between irinotecan, said irinotecan derivative or said pharmaceutically acceptable salt thereof and the lipids comprised in the bilayer is at least 0.18, within one week avoiding freezing. Even more attractive results were obtained for thermosensitive liposome according to the invention comprising irinotecan, an irinotecan derivative or a pharmaceutically acceptable salt thereof as an active pharmaceutical ingredient, wherein the pH of the intraliposomal buffer is from 5.0 up to 8.0 and the molar ratio between irinotecan, said irinotecan derivative or said pharmaceutically acceptable salt thereof and the lipids comprised in the bilayer is at least 0.20, within one week avoiding freezing.

### Example 8: Vesicle Size Affects Long-Term Storage Stability of DPPG₂-TSL-API

DPPG₂-TSL_{30%}-DOX have been prepared according to the large-scale method described in example 1 differing in vesicle size from 85 nm to 120 nm. DOX was loaded using an intraliposomal ammonium phosphate pH 7.4 buffer. Two different molar API:lipid ratios (0.06 & 0.08) were additionally tested. After production, the batches were stored at 5±3° C. for up to 15 weeks and biophysical characterization was performed, investigating vesicle size, PDI, total lipid excipient content (DPPC, DSPC, DPPG₂, without including peak areas of lipid-related decomposition products) by HPLC/CAD, total DOX (without including peak areas of DOX-related decomposition products) by HPLC, background fluorescence as indicator for DOX leakage, and TDR profile in FCS.

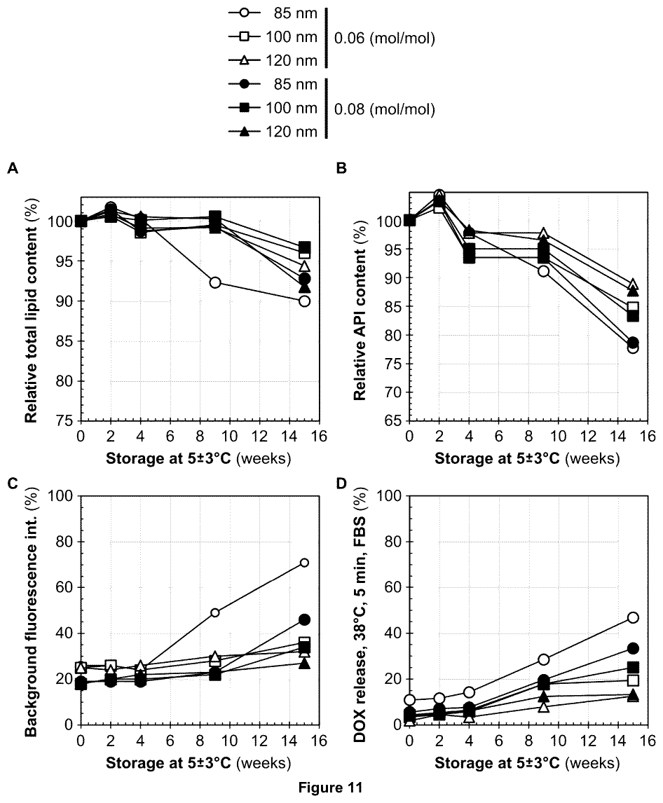

Batches with a vesicle size of 85 nm showed insufficient dispersion stability with significant increase in polydispersity after 4 weeks of storage, making DLS measurements for later stability time points impossible. For all other tested vesicle sizes, the z average and PDI only gradually increased during the storage period, showing sufficient stability if vesicle sizes 100 nm are used. Reducing vesicle size affected surprisingly API stability during storage independent from molar API:lipid ratio (FIG. 11B). API content in DPPG₂-TSL_{30%}-DOX with a vesicle size of 85 nm, 100 nm, and 120 nm was reduced to 78.2%, 84.1%, and 88.3% when stored 15 weeks at 5±3° C., respectively. For total lipid excipient content an increased decomposition was visible for the 85 nm vesicles for DPPG₂-TSL_{30%}-DOX with a molar API:lipid ratio of 0.06 (FIG. 11A). The smallest DPPG₂-TSL_{30%}-DOX with a vesicle size of 85 nm also showed a notable DOX leakage during storage, which started after 4 and 9 weeks for formulations with a molar API:lipid ratio of 0.06 and 0.08 (FIG. 11C), respectively. The effect of decreasing vesicle size on decreased store-ability of DPPG₂-TSL_{30%}-DOX was also detectable by an increase in DOX release at 38° C. in FCS when stored longer than 4 weeks at 5±3° C. (FIG. 11D). Overall, reducing vesicle size to a z average <100 nm, the formulation is prone to API and lipid excipient decomposition which in turn negatively affects the TDR profile. It is known, that decreasing vesicle size of in DPPG₂-TSL_{30%} increases water exchange across the lipid excipient bilayer at temperatures <Tₘ. This increased permeability for water increases the risk for hydrolytic decomposition processes in the formulation. If the API is prone to such decomposition processes, a too small vesicle size of DPPG₂-TSL has to be avoided. Moreover, one DOX-related impurity which is generated during long-term storage at 2-8° C. in the DPPG₂-TSL_{30%}-DOX is highly lipophilic and might therefore be responsible in the change in TDR profile during storage. Since the DOX crystal is in spatial proximity to the lipid excipient bilayer (FIG. 10), this might yield to lipid excipient-DOX adducts.

In conclusion, attractive results were obtained for a thermosensitive liposomes according to the invention comprising doxorubicin, a doxorubicin derivative or a pharmaceutically acceptable salt thereof, wherein the pH of the intraliposomal buffer is from 5.0 up to 8.0, wherein the molar ratio between doxorubicin, said doxorubicin derivative or said pharmaceutically acceptable salt thereof and the lipids comprised in the bilayer is from 0.06 up to 0.10, wherein the diameter of said thermosensitive liposome is from 100 nanometers up to 200 nanometers, within one week avoiding freezing. Even more attractive results were obtained for a thermosensitive liposomes according to the invention comprising doxorubicin, a doxorubicin derivative or a pharmaceutically acceptable salt thereof, wherein the pH of the intraliposomal buffer is from 6.4 up to 8.0, wherein the molar ratio between doxorubicin, said doxorubicin derivative or said pharmaceutically acceptable salt thereof and the lipids comprised in the bilayer is from 0.07 up to 0.09, wherein the diameter of said thermosensitive liposome is from 100 nanometers up to 150 nanometers, within four weeks avoiding freezing.

### Example 9: Salt Content in Storage Buffer Affects Long-Term Storage Stability of DPPG₂-TSL-API

Since DPPG₂-TSL_{30%}-DOX are prone to API decomposition when stored as liquid dispersion at 2-8° C., it was decided to store the formulation as frozen drug product at −20° C. For this purpose, a cryoprotectant was added to extraliposomal buffer to allow multiple freezing and thawing cycles (FTC) of the drug product without change in quality critical specifications. Disaccharides like e.g. trehalose are optimal cryoprotectants for liposomal formulations. To investigate effect of additional buffer salts to maintain pH value around neutral pH, distinct DPPG₂-TSL_{30%}-DOX with a vesicle size of −120 nm and a molar API:lipid ratio of 0.08 have been prepared according to the large-scale method described in example 1.

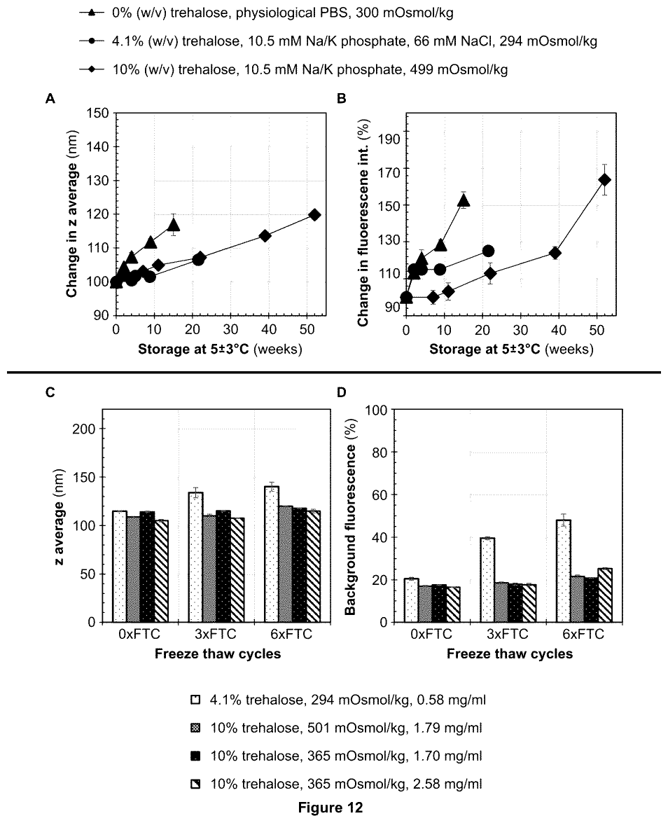

Reducing saline concentration in the storage buffer from physiological concentration (140 mM) to ≤66 mM stabilized the DPPG₂-TSL_{30%}-DOX for storage at 2-8° C., resulting in a dispersion that showed a notably slower increase in vesicle size (FIG. 12A). To adjust osmolarity to physiological concentration, non-ionic trehalose was added to the storage buffer. The Na/K phosphate buffer salt concentration was kept constant (10.5 mM). Also, the API leakage rate during storage was gradually suppressed, when saline concentration was reduced from 240 mM to 0 mM (FIG. 12B).

To achieve freezing and thawing stability for DPPG₂-TSL_{30%}-DOX 8% (w/v) of trehalose is needed in the storage buffer. A storage buffer containing 10% (w/v) trehalose and 10.5 mM Na/K phosphate pH 7.4 resulted in a liposomal formulation that survived 6 FTC without a notable change in vesicle size (FIG. 12C) or DOX leakage (FIG. 12D). Beside trehalose, also sucrose was successfully tested to achieve freezing and thawing stability of DPPG₂-TSL_{30%}-DOX (data not shown).

In conclusion, attractive results are obtained for a thermosensitive liposome according to the invention wherein said thermosensitive liposome is dissolved in a storage buffer with a saline concentration up to 100 mM and an osmolarity of at least 300 mOsmol/kg. Even more attractive results are obtained for a thermosensitive liposome according to the invention wherein said thermosensitive liposome is dissolved in a storage buffer with a saline concentration up to 20 mM and an osmolarity of at least 300 mOsmol/kg.

### Example 10: Final Formulation for DPPG₂-TSL_{30%}-DOX

DPPG₂-TSL_{30%}-DOX intended for (pre)clinical development have been prepared according to the large-scale preparation method described in example 1 (FIG. 2). All solutions were prepared with deionized and purified water from an ultrapure water system and subsequently filtered through 0.2 μm before usage. Phospholipids in molar ratio DPPC/DSPC/DPPG₂ 50:20:30 were dissolved in ethanol at 60° C. 300 mM ammonium phosphate buffer was prepared in ultrapure water, the pH was adjusted to 7.4 with phosphoric acid and the buffer was filtered through 0.2 μm before usage. PBS buffer was prepared in ultrapure water and pH was adjusted to 7.4 with phosphoric acid if required. The PBS buffer was also filtered through a 0.2 μm before usage. The ethanolic lipid solution and the ammonium phosphate buffer were pumped through two heating coils (60° C.) and the streams were joined at 60° C. using a T joint. Subsequently, the 60° C. hot dispersion was pumped through a 60° C. hot extrusion chamber equipped with multiple extrusion membranes of appropriate pore size to generate liposomes with a vesicle size in the range of 100 to 150 nm. After extrusion, the stream was pumped through a 5° C. cold cooling coil into an ice-cooled collector vessel. The gradient for active loading of DOX was generated by an extraliposomal buffer exchange to PBS pH 7.4 with TFF. The dispersion was subsequently filtered through 0.2 μm membranes to reduce bioburden. The desired molar API:lipid ratio of 0.08 was obtained by mixing the liposomal dispersion with PBS buffer pH 7.4 and the API solution (5.7 mg/ml DOX HCl in ultrapure water). The dispersion was heated under stirring at 37° C. for 30 min in a round bottom flask equipped with a heating mantle. Subsequently, traces of unencapsulated DOX were removed and extraliposomal buffer was exchanged to 10% (w/v) trehalose, 10.5 mM Na/K phosphate by TFF. Finally, the dispersion underwent aseptic filtration (0.2 μm). TSL preparations have been characterized as described in example 1. Turning point was calculated by fitting the TDR profile with a sigmoid function from 37° C. to 43° C. Residual solvents were measured with gas chromatography (headspace). Additionally, pH, osmolarity, bacterial endotoxin, and sterility were measured with the corresponding European Pharmacopeia (EP) methods. Representative analytical results of the independent manufactured batches are depicted in Table 3.

TABLE 3
Representative analytical results of DPPG₂-TSL_{30%}-DOX batches obtained with the
large-scale preparation method. Batch 1 and Batch 3 showed larger amounts of API-
related impurities, because incubation time during DOX loading was 120 and 90 minutes
instead of 30 minutes, respectively. For batch 4, optimized HPLC methods with notably
higher specifity and selectivity were used for lipid and DOX content, resulting
in detection of more and higher levels of impurities. TDRI: total amount of drug-
related impurities, TLRI: total amount of lipid-related impurities.

| Test | Batch 1 | | Batch 2 | | Batch 3 | | Batch 4 | |
|---|---|---|---|---|---|---|---|---|
| Target amount | 1 | L | 1 | L | 4 | L | 2.5 | L |
| DOX concentration | low | | high | | high | | medium | |
| Identity Lipids & API (HPLC) | Complies | | Complies | | Complies | | Complies | |
| Content (HPLC) | | | | | | | | |
| DPPC (mg/mL) | 3.15 | | 14.8 | | 14.8 | | 14.9 | |
| DSPC (mg/mL) | 1.32 | | 6.6 | | 6.4 | | 6.4 | |
| DPPG₂ (mg/mL) | 2.10 | | 9.2 | | 10.2 | | 9.8 | |
| Doxorubicin (mg/mL) | 0.36 | | 1.9 | | 1.82 | | 1.3 | |
| TLRI | Not detected | | <0.10% | a/a | 0.13% | m/m | 4.4% | a/a |
| TDRI (% a/a) | 3.9% | | <0.10% | | 4.1% | | 2.9 | |
| Residual EtOH (ppm m/m) | <500 | | <500 | | <500 | | <300 | |
| pH (EP 2.2.3) | 7.2 | | 7.4 | | 7.4 | | 7.3 | |
| Osmolality (EP 2.2.35) (mOsmol/kg) | 499 | | 387 | | 390 | | 402 | |
| Particle size (nm) | 123 | | 122.4 | | 121.5 | | 130 | |
| Polydispersity index (PDI) | 0.098 | | 0.09 | | 0.081 | | 0.10 | |
| Turning point (° C.) | 40.4 | | 40.9 | | 40.2 | | 40.66 | |
| Bacterial endotoxin (EP 2.6.14) | <1.00 | IU/ml | <0.88 | IU/ml | <0.40 | IU/ml | <0.5 | EU/ml |

| | | | | |
|---|---|---|---|---|
| Sterility (EP 2.6.1) | Complies | Complies | Complies | Not tested |

The results of the 12 months stability study of DPPG₂-TSL_{30%}-DOX batch 1 stored at 5° C.±3° C. indicated that the stability of the batch was independent of the four different vial materials used for storage of the dispersion (glass, PP, COP and COC). Additionally, the same is true for DPPG₂-TSL_{30%}-DOX batch 1 stored at −20° C.±5° C. The results further indicated that DPPG₂-TSL_{30%}-DOX batch 1 was stable for 11 weeks when stored at 5° C.±3° C. After that time point, degradation of DOX as well as a small but steady increase in vesicle size was observed. No signs of phospholipid excipient decomposition were visible during the 12 months of storage.

With DPPG₂-TSL_{30%}-DOX batch 3, an 18-month (−20° C.±5° C., Table 4) QA/QC-controlled stability study was performed. Samples were analyzed with the same methods as described above. DPPG₂-TSL_{30%}-DOX was stable for at least 18 months.

TABLE 4
Storage stability study results with DPPG₂-TSL_{30%}-DOX batch 3 under
ICH condition at −20° C. ± 5° C.

| Time | Particle | | | | | | |
|---|---|---|---|---|---|---|---|
| point | size | | TDRI | TLRI | TP | BF | Sterility |
| (months) | (nm) | PDI | (% a/a) | (% a/a) | (° C.) | (%) | (EP 2.6.1) |
| 0 | 121.5 | 0.08 | 4.1 | 0.13 | 40.2 | 14.5 | Sterile |
| 3 | 124.4 | 0.09 | —^{a} | 0.35 | 40.6 | 13.4 | n.d. |
| 6 | 125.7 | 0.07 | 4.4 | 0.11 | 40.7 | 15.1 | Sterile |
| 9 | 124.4 | 0.07 | 6.8 | 0.35 | 40.2 | 18.6 | n.d. |
| 12 | 124.3 | 0.09 | 5.9 | 0.11 | 40.6 | 14.9 | n.d. |
| 15 | 124.6 | 0.08 | 4.8 | 0.24 | 40.2 | 20.2 | n.d. |
| 18 | 125.4 | 0.07 | 5.0 | 0.26 | 40.2 | 16.9 | n.d. |

BF: Background fluorescence, TDRI: total amount of drug-related impurities, TLRI: total amount of lipid-related impurities, TP: Turning point.
^{a}Assay result excluded by QA/QC due to a deviation by the operator during analysis.

The results of the freezing and thawing stability study indicated that DPPG₂-TSL_{30%}-DOX batch 1 was stable for 6 freezing and thawing cycles. Furthermore, the different vial materials have also no influence on the stability of the drug product during freezing and thawing. The study was repeated under QA/QC-control with DPPG₂-TSL_{30%}-DOX batch 4, since a method for measurement of non-liposomal (free) DOX using SPE-based sample preparation was available (Table 5). The batch was stable for at least 3 freezing and thawing cycles. DPPG₂-TSL_{30%}-DOX batch 4 was further used to measure bench top stability at room temperature under QA/QC-control (Table 6). The batch was stable for at least for 8 hours at ambient temperatures.

TABLE 5
Freezing and thawing stabilitry study results with DPPG₂
-TSL_{30%}-DOX batch 4. FTC: numbers of freezing and thawing cycles,
n.d.: not determined, TDRI: total amount of drug-related impurities,
TLRI: total amount of lipid-related impurities.

| | Particle | | | | Free |
|---|---|---|---|---|---|
| | size | | TDRI | TLRI | DOX |
| FTC | (nm) | PDI | (% a/a) | (% a/a) | (%) |
| 0 | 134 | 0.12 | 2.8 | 2.3 | 6.6 |
| 1 | 126 | 0.09 | 2.8 | 3.1 | 6.0 |
| 2 | 129 | 0.09 | n.d. | n.d. | n.d. |
| 3 | 129 | 0.10 | n.d. | n.d. | 9.3 |

TABLE 6
Bench top stability results (store-ability at ambient
temperatures) with DPPG₂-TSL_{30%}-DOX batch 4. n.d .: not
determined, TDRI: total amount of drug-related impurities,
TLRI: total amount of lipid-related impurities.

| | | | | | Free |
|---|---|---|---|---|---|
| Time point | Particle | | TDRI | TLRI | DOX |
| (h) | size (nm) | PDI | (% a/a) | (% a/a) | (%) |
| 0 | 134 | 0.12 | 2.8 | 2.3 | 6.6 |
| 4 | 127 | 0.10 | 3.0 | 3.1 | 4.3 |
| 8 | 126 | 0.10 | 2.8 | n.d. | 7.0 |

TABLE 7
Biodistribution study in Brown Norway rats with a s.c. BN175
sarcoma on a hind limb obtained with DPPG₂-TSL_{30%}-DOX (small
lab scale vs. large scale manufacturing method). DOX concentration
(ng/mg) in selected organs and tumor tissues.

| | Small lab scale | Large-scale manufacturing |
|---|---|---|
| | manufacturing method | method |
| tumor with HT | 47.5 ± 25.4 | 49.2 ± 4.3 |
| tumor without HT | 3.0 ± 0.9 | Not measured |
| heart | 4.5 ± 0.8 | 3.7 ± 0.5 |
| liver | 13.2 ± 2.0 | 14.7 ± 2.8 |
| spleen | 18.3 ± 2.9 | 19.9 ± 0.6 |
| kidney | 19.6 ± 2.9 | 16.9 ± 2.5 |

A BD study was carried out to bridge DPPG₂-TSL_{30%}-DOX produced with the small lab scale method and the large-scale manufacturing method (batch 1). For the study, −250-300 gram Brown Norway rats were s.c. implanted with a 1 mm 3 BN175 sarcoma tumor fragment on each hind limb, 7-10 days prior to the experiment. Once one tumor reached 1 cm 3, it was subjected to a 1 h HT+DPPG₂-TSL_{30%}-DOX treatment (2 mg DOX per kg bodyweight). In short, a temperature measurement probe was placed inside the core of the tumor, followed by positioning of a heating lamp for bringing the tumor to 41° C. which averagely took 20 min to reach this target temperature. Once the tumor reached 41° C., the formulations were i.v. administered via a tail vein catheter and the tumor was heated for an additional hour. After the treatment, tumors, heart, liver, spleen and kidneys were excised and DOX concentration was measured (Table 7), using HPLC. Tissue levels of DOX revealed no difference in the BD profile between both tested formulations. The standard deviation for the heated tumors treated with DPPG₂-TSL_{30%}-DOX produced by the large-scale method was higher due to a broader range of tumor sizes (expressed by the measured tumor weight) investigated in the present study. Tumor weight was 651±264 mg compared to 661±120 mg, respectively. The underlying effect of tumor size on DOX accumulation efficacy was previously shown [6]. Animals treated with DPPG₂-TSL_{30%}-DOX produced by the scale method showed a mean intra-individual DOX enhancement ratio in the heated tumor relative to the non-heated of 47.5/3.0=15.8. This is in the range to the ratio obtained with state-of-the art DPPG₂-TSL_{30%}-DOX produced with 300 mM citrate pH 4 as intraliposomal buffer were the enhancement ratio ranged from 13 to 17 [6], indicating that the stabilization of the formulation by change in e.g. excipients, API:lipid ratio, vesicle size (see examples above) had no negative influence on the therapeutic efficacy.

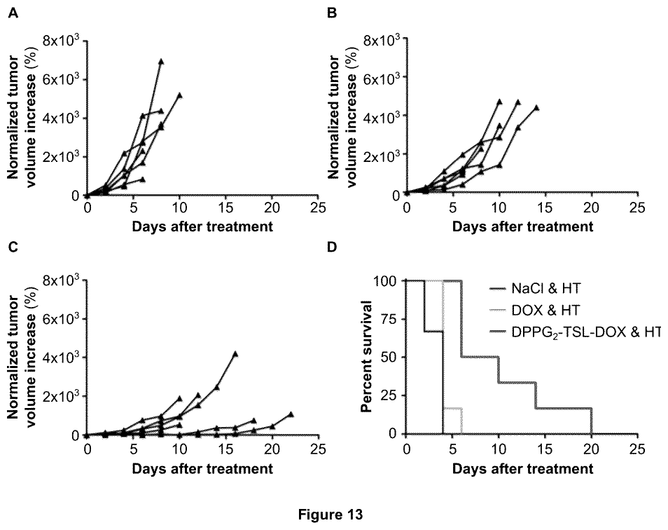

Therapeutic studies were performed with a similar BN175 tumor (5 mm in diameter; in any dimension) and treatment model as stated above. Different to the BD study, only a single tumor was implanted on the hind limb to avoid necessity to terminate the experiment due to uncontrolled growth of the untreated tumor. Beside DPPG₂-TSL_{30%}-DOX batch 1, animals have been treated with a non-liposomal DOX formulation (drug product approved for application in humans) and physiological saline (0.9% NaCl). All animal received additionally 1-hour local HT (41° C.) of the tumor as described above for the BD study. After treatment, tumors were measured by caliper every other day and animals were sacrificed when tumors reached 2.5 mm³ in volume or when ulceration was prominent. Treatment of rats with DPPG₂-TSL_{30%}-DOX showed significantly improved tumor growth delay versus animals treated with saline (p<0.01, FIG. 13). Furthermore, compared to the non-liposomal DOX treatment at equivalent dose, DPPG₂-TSL_{30%}-DOX also yielded significant tumor growth delay and prolonged survival (p<0.05, FIG. 13).

1. A thermosensitive liposome comprising a bilayer and an intraliposomal buffer,
wherein the molar concentration of 1,2-dipalmitoyl-sn-glycero-3-phosphodiglycerol (DPPG2) in said bilayer is at least 15 percent,
wherein said thermosensitive liposome comprises an active pharmaceutical ingredient, and
wherein the molar ratio between said active pharmaceutical ingredient and the lipids comprised in said bilayer is from 0.05 up to 0.3.

2. A thermosensitive liposome according to claim 1, wherein said active pharmaceutical ingredient is comprised in said intraliposomal buffer.

3. A thermosensitive liposome according to claim 2,
wherein said active pharmaceutical ingredient is doxorubicin, a doxorubicin derivative or a pharmaceutically acceptable salt thereof,
wherein the molar ratio between doxorubicin, said doxorubicin derivative or said pharmaceutically acceptable salt thereof and the lipids comprised in said bilayer is from 0.06 up to 0.10.

4. A thermosensitive liposome according to claim 2,
wherein said active pharmaceutical ingredient is irinotecan, an irinotecan derivative or a pharmaceutically acceptable salt thereof,
wherein the molar ratio between irinotecan, said irinotecan derivative or said pharmaceutically acceptable salt thereof and the lipids comprised in said bilayer is at least 0.18.

5. A thermosensitive liposome according to claim 2,
wherein said active pharmaceutical ingredient is gemcitabine, a gemcitabine derivative or a pharmaceutically acceptable salt thereof,
wherein the molar ratio between gemcitabine, said gemcitabine derivative or said pharmaceutically acceptable salt thereof and the lipids comprised in said bilayer is at least 0.12.

6. A thermosensitive liposome comprising a bilayer and an intraliposomal buffer,
wherein the molar concentration of 1,2-dipalmitoyl-sn-glycero-3-phosphodiglycerol (DPPG2) in said bilayer is at least 15 percent,
wherein said thermosensitive liposome comprises an active pharmaceutical ingredient, preferably wherein said intraliposomal buffer has a pH from 5 up to 8, preferably from 6 up to 8,
preferably wherein the molar ratio between said active pharmaceutical ingredient and the lipids comprised in said bilayer is from 0.05 up to 0.3,
more preferably wherein
said active pharmaceutical ingredient is doxorubicin, a doxorubicin derivative or a pharmaceutically acceptable salt thereof, even more preferably wherein the molar ratio between said doxorubicin, said doxorubicin derivative or said pharmaceutically acceptable salt thereof and the lipids comprised in said bilayer is from 0.06 up to 0.10, most preferably from 0.07 up to 0.09, or
said active pharmaceutical ingredient is irinotecan, an irinotecan derivative or a pharmaceutically acceptable salt thereof, even more preferably wherein the molar ratio between said irinotecan, said irinotecan derivative or said pharmaceutically acceptable salt thereof and the lipids comprised in said bilayer is at least 0.18, most preferably at least 0.20, or
said active pharmaceutical ingredient is gemcitabine, a gemcitabine derivative or a pharmaceutically acceptable salt thereof, even more preferably wherein the molar ratio between said gemcitabine, said gemcitabine derivative or said pharmaceutically acceptable salt thereof and the lipids comprised in said bilayer is at least 0.12, most preferably at least 0.15.

7. A composition comprising a thermosensitive liposome dispersed in a storage buffer,
wherein the saline concentration of said storage buffer is lower than 100 mM and the osmolarity of said storage buffer is higher than 300 mOsmol/kg;
wherein said thermosensitive liposome comprises a bilayer and an intraliposomal buffer;
wherein the molar concentration of 1,2-dipalmitoyl-sn-glycero-3-phosphodiglycerol (DPPG2) in said bilayer is at least 15 percent;
wherein said thermosensitive liposome comprises an active pharmaceutical ingredient;
preferably wherein said intraliposomal buffer has a pH from 5 up to 8, preferably from 6 up to 8;
preferably wherein the molar ratio between said active pharmaceutical ingredient and the lipids comprised in said bilayer is from 0.05 up to 0.3,
more preferably wherein
said active pharmaceutical ingredient is doxorubicin, a doxorubicin derivative or a pharmaceutically acceptable salt thereof, even more preferably wherein the molar ratio between said doxorubicin, said doxorubicin derivative or said pharmaceutically acceptable salt thereof and the lipids comprised in said bilayer is from 0.06 up to 0.10, most preferably from 0.07 up to 0.09, or
said active pharmaceutical ingredient is irinotecan, an irinotecan derivative or a pharmaceutically acceptable salt thereof, even more preferably wherein the molar ratio between said irinotecan, said irinotecan derivative or said pharmaceutically acceptable salt thereof and the lipids comprised in said bilayer is at least 0.18, most preferably at least 0.20, or
said active pharmaceutical ingredient is gemcitabine, a gemcitabine derivative or a pharmaceutically acceptable salt thereof, even more preferably wherein the molar ratio between said gemcitabine, said gemcitabine derivative or said pharmaceutically acceptable salt thereof and the lipids comprised in said bilayer is at least 0.12, most preferably at least 0.15; and
preferably wherein said composition comprises at least one excipient that may further aid in enhancing the delivery of said composition and/or said thermosensitive liposome to a tissue and/or cell and/or into a tissue and/or cell.

8. A thermosensitive liposome according to claim 1, wherein the concentration of said DPPG2 in said bilayer is from 15 mol % up to 35 mol %.

9. A thermosensitive liposome according to claim 1, wherein said thermosensitive liposome has a diameter from 100 nanometers up to 200 nanometers.

10. A thermosensitive liposome according to claim 1, wherein said bilayer does not comprise cholesterol or a derivative thereof.

11. A thermosensitive liposome according to claim 1, wherein said 1,2-dipalmitoyl-sn-glycero-3-phosphodiglycerol (DPPG2) is represented by Formula (IV):

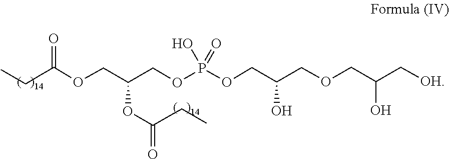

12. A thermosensitive liposome according to claim 1, wherein said bilayer comprises 1,2-dipalmitoyl-sn-glycero-3-phosphocholine (DPPC) and/or 1,2-distearoyl-sn-glycero-3-phosphocholine (DSPC).

13. A thermosensitive liposome according to claim 11,
wherein the molar concentration of 1,2-dipalmitoyl-sn-glycero-3-phosphocholine (DPPC) in said bilayer is from 0.45 up to 0.65; and/or
wherein the molar concentration of 1,2-distearoyl-sn-glycero-3-phosphocholine (DSPC) in said bilayer is from 0.15 up to 0.25; and/or
wherein the molar concentration of 1,2-dipalmitoyl-sn-glycero-3-phosphodiglycerol (DPPG2) in said bilayer is from 0.15 up to 0.35.

14. A thermosensitive liposome according to claim 1, for use as a medicament, preferably wherein said thermosensitive liposome or said composition is for use in treating, ameliorating, delaying, curing and/or preventing cancer.

15. A method for preparing a liposome, preferably a thermosensitive liposome according to comprising:
a) preparing an unloaded liposome, wherein said unloaded liposome comprises a bilayer with the same lipid composition as said liposome, and wherein said unloaded liposome does not comprise the active pharmaceutical ingredient comprised in said liposome,
ac) preferably extruding said unloaded liposome,
b) loading said unloaded liposome with active pharmaceutical ingredient by active loading using a loading buffer to form a loaded liposome, wherein said loading buffer has a salt concentration of at least 66 mM, preferably from 66 mM up to 120 mM, and an osmolarity from of at least 250 mOsmol/kg, preferably from 250 mOsmol/kg up to 350 mOsmol/kg, wherein in said loading is preferably carried out at a temperature from 35° C. to 39° C., more preferably from 36° C. to 38° C., most preferably from 37° C. to 38° C.;
c) exchanging said loading buffer by a storage buffer, wherein said storage buffer has a salt concentration lower than 100 mM and an osmolarity higher than 300 mOsmol/kg,
d) preferably storing said loaded liposome in said storage buffer from 1 up to 20 weeks at a temperature around 5° C., or from 1 up to 20 weeks at a temperature around 5° C., or from 1 up to 16 months at a temperature around −20° C., or from 12 up to 16 months at a temperature around −20° C.

16. A method for preparing a liposome according to claim 15, wherein said a) preparing of said unload liposome comprises:
aa) preparing a solution of lipids in an organic solvent with the same molar ratio as the lipids in the bilayer of said liposome, preferably wherein said organic solvent is chloroform/methanol 9:1 (vol/vol), chloroform or ethanol; and
ab) mixing said solution of lipids with an aqueous solution,
wherein said aqueous solution has essentially the same composition as the intraliposomal buffer comprised in said liposome, without the presence of said active pharmaceutical ingredient, preferably wherein said aqueous solution has a pH from 5 up to 8, preferably from 6 up to 8, and
preferably wherein said aqueous solution comprises a buffer selected from the group consisting of a (NH₄)2SO4 buffer, a (NH₄)2HPO4 buffer, a phosphate buffer and an HBS buffer, and
wherein said mixing is preferably performed via organic solvent injection, and
wherein said mixing leads to the formation of an unloaded liposome.

17. A composition according to claim 7, wherein the concentration of said DPPG2 in said bilayer is from 15 mol % up to 35 mol %, preferably from 20 mol % up to 30 mol %.

18. A composition according to claim 7, wherein said thermosensitive liposome has a diameter from 100 nanometers up to 200 nanometers, preferably from 100 nanometers up to 150 nanometers.

19. A composition according to claim 7, wherein said bilayer does not comprise cholesterol or a derivative thereof.

20. A composition according to claim 7, wherein said 1,2-dipalmitoyl-sn-glycero-3-phosphodiglycerol (DPPG2) is represented by Formula (IV): [chemical structure]
